(19) United States
(12) Patent Application Publication
(10) Pub. No.: US 2015/0018396 A1
(43) Pub. Date: Jan. 15, 2015

(54) PREVENTION AND TREATMENT OF RESPIRATORY INFECTION WITH PEROXISOME PROLIFERATOR ACTIVATOR RECEPTOR DELTA AGONIST

(71) Applicant: PRESIDENT AND FELLOWS OF HARVARD COLLEGE, Cambridge, MA (US)

(72) Inventors: Chih-Hao Lee, Boston, MA (US); Prerna Bhargava, Woodbridge, CT (US)

(21) Appl. No.: 14/382,710

(22) PCT Filed: Mar. 3, 2013
(86) PCT No.: PCT/US2013/029838

§ 371 Date: Sep. 3, 2014

Related U.S. Application Data
(60) Provisional application No. 61/608,318, filed on Mar. 8, 2012.

(51) Int. Cl. C07D 277/26

(52) U.S. Cl. CPC C07D 277/26
USPC 514/365

(57) ABSTRACT

The present disclosure is related generally to a method for prevention and/or treatment of infections, e.g., respiratory infections, in a subject before or after the onset of a disease state associated with the infection.

### RELATED APPLICATION

This application claims benefit under 35 U.S.C. §119(e) of the U.S. Provisional Application No. 61/608,318, filed Mar. 8, 2012, the content of which is incorporated herein by reference in its entirety.

### TECHNICAL FIELD

The present disclosure is related generally to a method for prevention and/or treatment of an infection, e.g., respiratory infection, in a subject before or after the onset of a disease state associated with the infection.

### BACKGROUND

There are about 2 million cases of pneumonia each year of which 40,000 to 70,000 result in death. The Merck Manual of Diagnosis and Therapy (17th ed. 1999). Although certain viruses and fungi cause pneumonia, most cases of pneumonia in adults are caused by bacteria such as Streptococcus pneumonia, Staphylococcus aureus, Haemophilus influenzae, Chlcanda pneumoniae, C. psittaci, C. trachomatis, Moraxella (Branhamella) catarrhalis, Legionella pneumophila, Klebsiella penumoniae, and other gram-negative bacilli. Id. Bacterial infections are the leading cause of death in children and the elderly and can lead to life-threatening complications in patients with chronic diseases, such as type 2 diabetes. (Huang, S. S., Johnson, K. M., Ray, G. T., Wroe, P., Lieu, T. A., Moore, M. R., Zell, E. R., Linder, J. A., Grijalva, C. G., Metlay, J. P. and Finkelstein, J. A. (2011) Healthcare utilization and cost of pneumococcal disease in the United States. Vaccine. 29, 3398-3412)

Pneumonia, induced by Streptoccocus pneumoniae (S. pneumoniae) infection, is the eighth leading cause of death in the United States. Over the last decade, over prescription of antibiotics has led to the emergence of antibiotic resistant bacteria that are exceedingly difficult to treat. Additionally, the increased susceptibility to infection of elderly patients and the rising healthcare costs associated with infections have necessitated the development of novel therapeutics to help fight bacterial infections

Pneumonia is diagnosed based on characteristic symptoms and an infiltrate on chest x-ray. Common symptoms of pneumonia include cough, fever, sputum production, tachypnea, and crackles with bronchial breath sounds. Id. Determination of the specific pathogen causing the pneumonia cannot be made in about 30-50% of patients and specimens can be misleading because of normal flora can contaminate samples through the upper airways. Special culture techniques, special stains, serologic assays, or lung biopsies can be used for diagnosis.

Current therapies for the treatment of pneumonia consist of respiratory support, such as oxygen, and antibiotics based on determination of the specific bacteria and/or according to the patient's age, epidemiology, host risk factors, and severity of illness. Id. For example, in cases of Staphylococcal pneumonia, anti-bacterial therapy comprises administration of penicillin (e.g., oxacillin and nafcillin), or cephalosporin (e.g. cephalothin or cefamandol, cefazolin, and cefuroxime). Id. In cases of streptococcal pneumonia, anti-bacterial therapy comprises administration of penicillin, cephalosporins, erythromycin, or clindamycin.

Currently, treatments for gram-positive bacteria include various antibiotics. Gram-negative bacteria have very few treatment options. The administration of antibiotics can result in side effects, toxicity, and the development of antibiotic resistant strains. In addition, because the pathogen causing pneumonia is difficult to diagnose, the use of antibiotics can be ineffective since both viruses and fungi also cause pneumonia.

Thus there is a need in the art for therapeutics to improve the ability of the host to kill the pathogen, e.g., treatment of pneumonia. This can help to reduce the dependence on antibiotic treatment with the hopes of reducing the high rate of generation of antibiotic resistant strains and increasing the outcome of survival in susceptible patients.

### SUMMARY

In one aspect the disclosure provides a method of treating, ameliorating, or preventing an infection in a subject. Generally the method comprises administering to a subject in need thereof a therapeutically effective amount of a PPAR agonist.

In some embodiments, the method is for preventing or treating an infection in a child or infant. The method comprising administering to the infant or child in need thereof a therapeutically effective amount a PPAR agonist.

In some embodiment, the method is for preventing or treating an infection in an immune compromised subject. The method comprising administering to a subject in need thereof a therapeutically effective amount of a PPAR agonist.

In some embodiments, the infection to be prevented and/or treated is upper respiratory tract infections caused by viruses and/or bacteria, e.g., a respiratory infection. In some embodiments, the infection is a peritoneum, urinary tract, or gut infection.

Another aspect of the present invention provides prophylactic and therapeutic protocols for the prevention, treatment, management, or amelioration of a respiratory condition comprising the administration of an effective amount of one or more PPAR agonists to a subject in need thereof.

The methods disclosed herein also encompass prophylactic and therapeutic protocols for the prevention, treatment, management, or amelioration of a respiratory condition comprising the administration of an effective amount of one or more PPAR agonists and an amount of at least one other therapy (e.g., prophylactic or therapeutic agent) other than a PPARδ or PPARγ agonist.

In some embodiments, the agonist is a PPARδ or a PPARγ agonist.

In some embodiments, the disclosure provides employing a compound of formula (I) or a pharmaceutically acceptable salt, solvate, or hydrolyzable ester thereof as the PPAR agonist in a method disclosed herein:

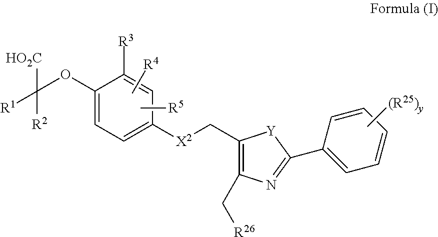

wherein:
R¹ and R² are independently hydrogen or C₁₋₃alkyl;
X² is O, S, or CH₂;
R³, R⁴, and R⁵ are independently H, C₁₋₃alkyl, OCH₃, CF₃, OCF₃, CN, allyl, or halogen;
Y is S or O;
each R²⁵ is independently CH₃, OCH₃, CF₃, or halogen;
each R²⁶ is independently for each occurrence

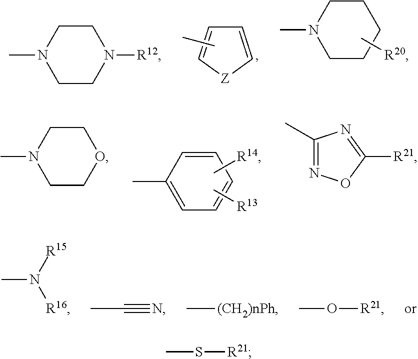

R¹² is independently for each occurrence C₁₋₆alkyl, C₁₋₆alkylenearyl,

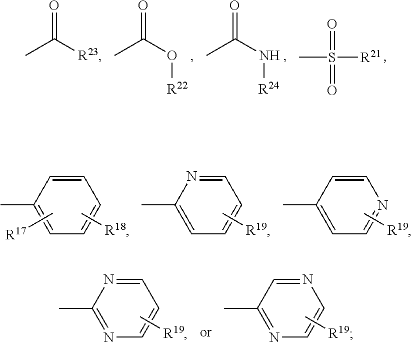

R¹³ and R¹⁴ are independently hydrogen, halogen, CN, perfluoroC₁₋₆alkyl, perfluoro-O—C₁₋₆alkyl, C₁₋₆alkyl, —OC₁₋₆alkyl, —C₁₋₆alkyleneOC₁₋₆alkyl, —SC₁₋₆alkyl, or aryl;
R¹⁵ and R¹⁶ are independently hydrogen, C₁₋₆alkyl, C₃₋₆cycloalkyl optionally substituted with 1 or 2 C₁₋₃alkyl groups, or R¹² as defined above;
R¹⁷ and R¹⁸ are independently hydrogen, halogen, hydroxy, —CN, C₁₋₆alkyl, C₁₋₆ perfluoroalkyl, C₁₋₆acyl, —OC₁₋₆alkyl, perfluoroOC₁₋₆alkyl, or C₁₋₆hydroxyalkyl;
R¹⁹ is independently for each occurrence hydrogen or C₁₋₆alkyl;
R²⁰ is independently for each occurrence C₁₋₆alkyl, aryl, —OC₁₋₆alkyl, hydroxy, C₁₋₆ hydroxyalkyl, or 1-alkoxyC₁₋₆alkyl;
R²¹ is independently for each occurrence C₁₋₆alkyl, —C₁₋₆alkylenearyl, aryl, or aryl-heteroaryl;
R²² is independently for each occurrence independently for each occurrence C₁₋₆alkyl, aryl, or —C₁₋₆alkylenearyl;
R²³ is C₁₋₆alkyl, C₃₋₆cycloalkyl, or aryl;
R²⁴ is independently for each occurrence C₁₋₆alkyl, C₁₋₆alkylenearyl, C₃₋₆cycloalkyl, or aryl;
Z is independently for each occurrence O, N, or S (note that when Z is N, the depicted bond can be attached to the nitrogen in the ring as well as any of the carbons in the ring);
y is 0, 1, 2, 3, 4, or 5; and
n is 1, 2, or 3.

### BRIEF DESCRIPTION OF THE DRAWINGS

FIGS. 1a-1e show defective bacteria clearance in myeloid-specific Ppar-δ knockout (Ppar-δ^{mye-/-}) mice in a mouse model of Pneumonia. FIG. 1a shows the survival curve of wt and Ppar-δ^{mye-/-} mice infected intranasally with 10⁵ CFU of S. pneumoniae. FIG. 1b shows the in vivo bacterial killing assay. Mice were infected with S. pneumoniae through nasal inhalation. Twenty-four hours after infection, bacterial counts (presented as % of initial CFU) in bronchoalveolar lavage fluid (Balf) were determined. FIG. 1c shows cell counts obtained from Balf of wt and Ppar-δ^{mye-/-} mice 24 hours after infection (left panel) and immune cell populations in Balf of infected mice (right panel). Mac: macrophage; Neutro: neutrophil; Lymph: lymphocyte. FIG. 1d shows the levels of Tnf-α and Il-6 in Balf measured 24 hours post infection. FIG. 1e shows the Immune cell populations in peripheral blood of infected mice. Data are presented as mean±SEM. *p<0.05.

FIGS. 2a-2e show the role of macrophage Ppar-δ in bacterial killing. FIG. 2a shows the In vitro bacterial clearance assay in wt and Ppar-δ^{-/-} macrophages. Bone marrow derived macrophages were infected with S. pneumoniae (10 bacteria: 1 macrophage) for 1 hour. Bacterial uptake and binding of macrophages were determined after extensive washing to remove unbound bacteria. Intracellular bacteria counts were determined for an additional 1 hour (after the initial incubation) to examine the bacterial killing activity of macrophages. FIG. 2b shows the phagocytosis assay. Macrophages were infected with heat-killed S. pneumoniae labeled with Alexa Fluor 647 (25 bacteria: 1 macrophage) for the indicated times and fluorescence was determined by FACS. FIG. 2c shows the phagosome numbers in wt and Ppar-δ^{-/-} macrophages 1 hour after infection with heat-killed Alexa Fluor 647: S. pneumoniae. The phagosome number was determined by dividing the mean fluorescence intensity (MFI) of the macrophage (with ingested bacteria) by the MFI of a single bacterium. FIG. 2d shows the phagosome acidification assay. Acidification was determined by infecting macrophages with heat killed S. pneumoniae dually labeled with FITC (pH sensitive) and Alexa Fluor 647 (pH insensitive). FIG. 2e shows that the ROS production is blunted in Ppar-δ^{-/-} macrophages. Phagosomal oxidative burst was determined using FcOxyburst reagents. Data are presented as mean±SEM. *p<0.05.

FIGS. 3a-3c show the transcriptional regulation of macrophage phagosomal function by Ppar-δ. FIGS. 3a and 3b show the expression of genes involved in NADPH oxidase complexes in cultured macrophages and alveolar macrophages, respectively. Gene expression was determined by real-time PCR. FIG. 3c shows that Ppar-δ regulates the promoter activity of Noxo1. RAW264.7 macrophages were transfected with a luciferase reporter driven by the 2 kb 5′-regulatory region of the Noxo1 or Nox2 gene. Twenty-four hours after transfection, cells were infected with S. pneumoniae or treated with GW501516 (GW1516), a synthetic Ppar-δ agonist, for 12 hours. FIG. 3d shows that the ability of GW501516 to increase Noxo1 promoter activity is lost in Ppar-δ^{-/-} macrophages. Transfection was done in stably transformed wt and Ppar-δ^{-/-} macrophage cell lines. Data are presented as mean±SEM. *p<0.05.

FIGS. 4a-4f show that Noxo1 expression rescues the decreased bacterial killing activity in Ppar-δ^{-/-} macrophages. FIG. 4a shows that Noxo1 is over-expressed in bone marrow derived macrophages from CSS 17 mice. FIG. 4b shows the in vitro killing assay showing macrophages from CSS 17 mice are more efficient in clearing S. pneumoniae infection. FIG. 4c shows that phagosome ROS production is increased in macrophages from CSS17 mice compared to wt controls. ROS was determined using FcOxyburst. FIG. 4d shows Noxo1 mRNA levels in wt and Ppar-δ^{-/-} stable macrophage lines and in Ppar-δ^{-/-} macrophages with forced Noxo1 expression. FIGS. 4e and 4f show that Noxo1 over-expression normalizes bacterial killing and ROS production capacity in Ppar-δ^{-/-} macrophages, respectively. Data are presented as mean±SEM. *p<0.05.

FIGS. 5a-5d show that Ppar-δ activation improves survival in a mouse model of pneumonia. FIG. 5a shows the survival curve of wt mice treated with vehicle or GW501516 (Ppar-δ agonist). Mice were gavaged with GW501516 (4 mg/kg/day) daily for 3 days prior to and every other day after infection. Mice were infected with 5×10⁵ CFU of S. pneumoniae. FIG. 5b shows the in vivo bacteria clearance assay in wt mice treated with GW501516 for 3 days. Twenty-four hours post-infection, lavage fluid was isolated and analyzed for bacterial counts. FIG. 5c shows the gene expression analyses by real-time PCR using lavage fluid samples from vehicle or GW501516 treated mice. Mice were gavaged with GW501516 for three days. FIG. 5d shows that GW501516 treatment increases phagosome ROS production in alveolar macrophages in a Ppar-δ dependent manner. Mice were gavaged with GW501516 for 3 days. Alveolar macrophages were isolated and ROS was determined using FcOxyburst. Data are presented as mean±SEM. *p<0.05.

FIGS. 6a and 6b show in vitro killing assays. FIG. 6a shows the in vitro killing assays of gram-negative bacteria (DH5α strain) in wt and Ppar-δ^{-/-} macrophages. Y axis: % of bacteria survived (over the initial bacterial counts). FIG. 6b shows the bacterial killing assays in Ppar-δ^{-/-} macrophages and in Ppar-δ^{-/-} macrophages re-expressing Ppar-δ.

FIGS. 7a and 7b show that Ppar-δ expression (FIG. 7a) and transcriptional activation of Ppar-δ (FIG. 7b) is increased during S. pneumoniae infection. FIG. 7a shows that Ppar-δ expression is increased in macrophages during infection with S. pneumoniae. Expression of Ppar-δ and its target gene Slc25a20 was determined by RT-qPCR in macrophages infected with S. pneumoniae for the indicated time. FIG. 7b shows that the transcriptional activation of Ppar-δ is increased during S. pneumoniae infection. RAW264.7 cells were transfected with Gal4-Ppar-δ ligand binding domain along with a Gal4 binding site-containing luciferase reporter construct. Twenty-four hours after transfection, cells were infected with S. pneumoniae for 12 hours. GW501516 (GW1516), a synthetic Ppar-δ agonist, was included as a positive control. Data are presented as mean±SEM. *p<0.05.

FIGS. 8a and 8b show the role of macrophage Ppar-γ in bacterial killing. FIG. 8a shows the in vitro bacterial clearance assay in wt and Ppar-γ^{-/-} macrophages. Bone marrow derived macrophages were infected with S. pneumoniae (10 bacteria: 1 macrophage) for 1 hour. Bacterial uptake and binding of macrophages were determined after extensive washing to remove unbound bacteria. Intracellular bacteria counts were determined for an additional 1 hour (after the initial incubation) to examine the bacterial killing activity of macrophages. FIG. 8b shows the in vivo bacterial killing assay. Wild-type and myeloid specific Ppar-γ knockout (Ppar-γ^{mye-/-}) mice were infected with S. pneumoniae through nasal inhalation. Twenty-four hours after infection, bacterial counts (presented as % of initial CFU) in bronchoalveolar lavage fluid (Balf) were determined.

### DETAILED DESCRIPTION

Aspects of the invention disclosed herein are based on inventor's discovery that PPARδ and/or PPARγ enhance bacterial killing activity of myeloid cells, such as macrophages and neutrophils. The killing activity is enhanced against both gram-negative and gram-negative species. Without wishing to be bound by a theory, it is believed that PPARδ and/or PPARγ simply improves phagosome activity by regulating the expression of phagosome proteins. This provides novel therapeutic strategies for improving infection treatment outcomes in subjects. The methods and compositions disclosed herein can be used to enhance phagosome activity of macrophages and thereby improving or enhancing treatment outcomes in a subject.

Accordingly, the disclosure provides a method for treating, ameliorating, or preventing an infection in a subject. The method comprising administering to a subject in need thereof a therapeutically effective amount of a PPAR agonist.

In embodiments of the various aspects disclosed herein, the PPAR agonist can be a PPARδ or a PPARγ agonists. PPAR agonists are described in detail below.

The disclosure also provides prophylactic and therapeutic protocols for the prevention, treatment, management, or amelioration of a respiratory condition or one or more symptoms thereof. The method comprising administering to a subject in need thereof a therapeutically effective amount of a PPAR agonist.

In some embodiments, the disclosure provides a method for preventing, managing, treating, or ameliorating pneumonia, said method comprising administering to a subject in need thereof a prophylactically or therapeutically effective amount of one or more PPAR agonist.

The methods disclosed herein also encompass prophylactic and therapeutic protocols for the prevention, treatment, management, or amelioration of an infection, or a respiratory condition, the method comprising co-administrating an effective amount of one or more PPAR agonists and at least one other therapy, e.g., a therapy currently being used, have been used, or are known to be useful in the prevention, management, treatment, and/or amelioration of said infection, respiratory condition or one or more symptoms associated therewith. The therapy can be a prophylactic or treatment with a therapeutic agent. In some embodiments, at least one other therapy is a therapy using a modality other than a PPAR agonist, e.g., a non-PPAR agonist therapeutic agent.

In some embodiments, the method is for preventing, treating, managing, or ameliorating pneumonia, the method comprising administering to a subject in need thereof an effective amount of one or more PPAR agonist and an effective amount of one or more therapies (e.g., one or more prophylactic or therapeutic agents) that are currently being used, have been used, or are known to be useful in the prevention, management, treatment, and/or amelioration of pneumonia or one or more symptoms thereof.

In some embodiments, the subject is an immunocompromised or immunosuppressed subject. In some embodiments, the subject is an infant or child. In some embodiments, the subject is an elderly subject.

Infections include, but are not limited to, viral infections, bacterial infections, anthrax, parasitic infections, fungal infections, and prion infections. As used herein, the term “viral infection” generally encompasses infection of an animal host, particularly a human host, by one or more viruses. Thus, treating viral infection will encompass the treatment of a person who is a carrier of one or more specific viruses or a person who is diagnosed of active symptoms caused by and/or associated with infection by the viruses. A carrier of virus can be identified by any methods known in the art. For example, a subject can be identified as virus carrier on the basis that the subject is antiviral antibody positive, or is virus-positive, or has symptoms of viral infection. That is, “treating viral infection” should be understood as treating a subject who is at any one of the several stages of viral infection progression. In addition, “treating or preventing viral infection” will also encompass treating suspected infection by a particular virus after suspected past exposure to virus by e.g., blood transfusion, exchange of body fluids, bites, accidental needle stick, or exposure to patient blood during surgery, or other contacts with a person with viral infection that may result in transmission of the virus.

Viral infections include, but are not limited to, infection by Exemplary viral infections include, but are not limited to, infection with hepatitis A, HIV, HTLV-1, HTLV-II, influenza A, influenza B, respiratory syncytial virus (RSV), herpes simplex virus types 1 and 2 (HSV), varicella zoster virus (VZV), cytomegalovirus (CMV), Epstein-Barr virus (EBV), human herpes virus type 6 (HHV6), human herpes virus type 7 (HHV-7), human herpes virus type 8 (HHV-8), human papilloma virus infection, rotavirus, adenovirus, SARS virus. poliovirus, encephalomyocarditis virus (EMCV) and smallpox virus. Examples of (+) strand RNA viruses which can be targeted for inhibition include, without limitation, picornaviruses, caliciviruses, nodaviruses, coronaviruses, arteriviruses, flaviviruses, and togaviruses. Examples of picornaviruses include enterovirus (poliovirus 1), rhinovirus (human rhinovirus 1A), hepatovirus (hepatitis A virus), cardiovirus (encephalomyocarditis virus), aphthovirus (foot-and-mouth disease virus O), and parechovirus (human echovirus 22). Examples of caliciviruses include vesiculovirus (swine vesicular exanthema virus), lagovirus (rabbit hemorrhagic disease virus), “Norwalk-like viruses” (Norwalk virus), “Sapporo-like viruses” (Sapporo virus), and “hepatitis E-like viruses” (hepatitis E virus). Betanodavirus (striped jack nervous necrosis virus) is the representative nodavirus. Coronaviruses include coronavirus (avian infections bronchitis virus) and torovirus (Berne virus). Arterivirus (equine arteritis virus) is the representative arteriviridus. Togavirises include alphavirus (Sindbis virus) and rubivirus (Rubella virus). Finally, the flaviviruses include flavivirus (Yellow fever virus), pestivirus (bovine diarrhea virus), and hepacivirus (hepatitis C virus).

In some embodiments, the viral infections are selected from influenza infection, RSV infection, chronic hepatitis B, chronic hepatitis C, HW infection, HSV infection, VSV infection, and CMV infection.

Bacterial infections include, but are not limited to, streptococci, staphylococci, E. coli, pseudomonas. In one embodiment, bacterial infection is intracellular bacterial infection. Intracellular bacterial infection refers to infection by intracellular bacteria such as mycobacteria (tuberculosis), chlamydia, mycoplasma, listeria, and facultative intracellular bacteria such as staphylococcus aureus.

Parasitic infections include, but are not limited to, worm infections, in particular, intestinal worm infection.

In some embodiments, the infection is a respiratory infection. In some embodiments, the subject has a respiratory condition. In some embodiment, the subject is at risk of developing a respiratory condition. In some embodiments, the subject is in need of treatment of a S. pneumoniae infection.

Exemplary conditions or disorders associated with respiratory infection include, but at are not limited to respiratory conditions associated with environmental factor, such as allergies and asthma; viral respiratory infections, such as parainfluenza virus infections, respiratory syncytial virus infections, and avian and human metapneumovirus; bacterial respiratory infections, such as bacterial pneumonia, and tuberculosis; fungal respiratory infections such as systemic candidiasis, and cryptococcosis.

Allergies are disorders of the immune system in which the body reacts to innocuous substances by inducing the generation of large amounts of immunoglobulin E (IgE). In the presence of an allergen, IgE activates mast cells and promotes mast cell proliferation, infiltration, and/or degranulation that results in the release of histamines, leukotrienes, and cytokines which cause rhinitis, hives, redness, itchiness, watery eyes, skin rashes, bronchoconstriction (wheezing), coughing, and difficulty breathing. Common allergens include, but are not limited to, pollens, molds, dust (e.g., dust mites and dust mite waste), animal protein (e.g., dander, urine, oil from the skin), industrial chemicals, foods, medicines, feathers, and insects (e.g., insect stings, cockroaches, and insect waste).

Pollinosis, commonly known as hay fever, is generally induced by wind-borne pollens, including, but not limited to tree pollens (e.g., oak, elm, maple, alder, birch, juniper, and olive), grass pollens (e.g., Bermuda, timothy, sweet vernal, orchard, and Johnson), weed pollens (e.g., Russian thistle, English plantain, and ragweed), and airborne fungal spores. Symptoms of pollinosis include itchy nose, roof of the mouth, pharynx, and eyes, sneezing, runny nose, watery eyes, headaches, anorexia, depression, coughing, insomnia, and wheezing. Common therapies include administration of antihistamines, sympathomimetics, glucocorticoids, and systemic corticosteroids and allergen immunotherapy. Unfortunately, these therapies can cause side effects, such as hypertension and drowsiness or can not be effective.

Anaphylaxis is an acute allergic reaction that results when the allergen reaches the circulation. Common allergens are parenteral enzymes, blood products, .beta.-lactam antibiotics, allergen immunotherapy, and insect stings. Anaphylaxis is characterized by smooth muscle contraction that causes wheezing, vasodilation, pulmonary edema, and obstructive angioedema. If the reaction is prolonged, the subject can develop arrhythmias or cardiogenic shock. In severe cases, the patient can suffer from primary cardiovascular collapse without respiratory symptoms. Long-term immunotherapy is effective for preventing anaphylaxis from insect stings, but is rarely available for patients with drug or serum anaphylaxis. Immediate administration of epinephrine is the most common treatment for anaphylaxis, but can cause side effects including headache, tremulousness, nausea, and arrhythmias. Thus, new therapies for the prevention, treatment, management, and amelioration of allergic reactions are needed.

About 12 million people in the U.S. have asthma and it is the leading cause of hospitalization for children. The Merck Manual of Diagnosis and Therapy (17th ed., 1999).

Asthma is an inflammatory disease of the lung that is characterized by airway hyperresponsiveness (“AHR”), bronchoconstriction (i.e., wheezing), eosinophilic inflammation, mucus hypersecretion, subepithelial fibrosis, and elevated IgE levels. Asthmatic attacks can be triggered by environmental triggers (e.g. acarids, insects, animals (e.g., cats, dogs, rabbits, mice, rats, hamsters, guinea pigs, mice, rats, and birds), fungi, air pollutants (e.g., tobacco smoke), irritant gases, fumes, vapors, aerosols, or chemicals, or pollen), exercise, or cold air. The cause(s) of asthma is unknown. However, it has been speculated that family history of asthma (London et al., 2001, Epidemiology 12(5):577-83), early exposure to allergens, such as dust mites, tobacco smoke, and cockroaches (Melen et al., 2001, 56(7):646-52), and respiratory infections (Wenzel et al., 2002, Am J Med, 112(8):672-33 and Lin et al., 2001, J Microbiol Immuno Infect, 34(4):259-64) can increase the risk of developing asthma.

Current therapies are mainly aimed at managing asthma and include the administration of .beta.-adrenergic drugs (e.g. epinephrine and isoproterenol), theophylline, anticholinergic drugs (e.g., atropine and ipratorpium bromide), corticosteroids, and leukotriene inhibitors. These therapies are associated with side effects such as drug interactions, dry mouth, blurred vision, growth suppression in children, and osteoporosis in menopausal women. Cromolyn and nedocromil are administered prophylatically to inhibit mediator release from inflammatory cells, reduce airway hyperresponsiveness, and block responses to allergens. However, there are no current therapies available that prevent the development of asthma in subjects at increased risk of developing asthma. Thus, new therapies with fewer side effects and better prophylactic and/or therapeutic efficacy are needed for asthma.

Respiratory infections are common infections of the upper respiratory tract (e.g., nose, ears, sinuses, and throat) and lower respiratory tract (e.g., trachea, bronchial tubes, and lungs). Symptoms of upper respiratory infection include runny or stuffy nose, irritability, restlessness, poor appetite, decreased activity level, coughing, and fever. Viral upper respiratory infections cause and/or are associated with sore throats, colds, croup, and the flu. Examples of viruses that cause upper respiratory tract infections include rhinoviruses and influenza viruses A and B. Common upper respiratory bacterial infections cause and/or associated with, for example, whooping cough and strep throat. An example of a bacteria that causes an upper respiratory tract infection is Streptococcus.

Clinical manifestations of a lower respiratory infection include shallow coughing that produces sputum in the lungs, fever, and difficulty breathing. Examples of lower respiratory viral infections are parainfluenza virus infections (“PIV”), respiratory syncytial virus (“RSV”), and bronchiolitis. Examples of bacteria that cause lower respiratory tract infections include Streptococcus pneumoniae that causes pneumonococcal pneumonia and Mycobacterium tuberculosis that causes tuberculosis. Respiratory infections caused by fungi include systemic candidiasis, blastomycosis crytococcosis, coccidioidomycosis, and aspergillosis. Respiratory infections can be primary or secondary infections.

Current therapies for respiratory infections involve the administration of anti-viral agents, anti-bacterial, and anti-fungal agents for the treatment, prevention, or amelioration of viral, bacterial, and fungal respiratory infections, respectively. Unfortunately, in regard to certain infections, there are no therapies available, infections have been proven to be refractory to therapies, or the occurrence of side effects outweighs the benefits of the administration of a therapy to a subject. The use of anti-bacterial agents for treatment of bacterial respiratory infections can also produce side effects or result in resistant bacterial strains. The administration of anti-fungal agents can cause renal failure or bone marrow dysfunction and can not be effective against fungal infection in patients with suppressed immune systems. Additionally, the infection causing microorganism (e.g., virus, bacterium, or fungus) can be resistant or develop resistance to the administered therapeutic agent or combination of therapeutic agents. In fact, microorganisms that develop resistance to administered therapeutic agents often develop pleiotropic drug or multidrug resistance, that is, resistance to therapeutic agents that act by mechanisms different from the mechanisms of the administered agents. Thus, as a result of drug resistance, many infections prove refractory to a wide array of standard treatment protocols. Therefore, new therapies for the treatment, prevention, management, and/or amelioration of respiratory infections and symptoms thereof are needed.

Parainfluenza viral (“PIV”) infection results in serious respiratory tract disease in infants and children. (Tao et al., 1999, Vaccine 17: 1100-08). Infectious parainfluenza viral infections account for approximately 20% of all hospitalizations of pediatric patients suffering from respiratory tract infections worldwide. Id.

PIV is a member of the paramyxovirus genus of the paramyxoviridae family. PIV is made up of two structural modules: (1) an internal ribonucleoprotein core or nucleocapsid, containing the viral genome, and (2) an outer, roughly spherical lipoprotein envelope. Its genome is a single strand of negative sense RNA, approximately 15,456 nucleotides in length, encoding at least eight polypeptides. These proteins include, but are not limited to, the nucleocapsid structural protein (NP, NC, or N depending on the genera), the phosphoprotein (P), the matrix protein (M), the fusion glycoprotein (F), the hemagglutinin-neuraminidase glycoprotein (HN), the large polymerase protein (L), and the C and D proteins of unknown function. Id.

The parainfluenza nucleocapsid protein (NP, NC, or N) consists of two domains within each protein unit including an amino-terminal domain, comprising about two-thirds of the molecule, which interacts directly with the RNA, and a carboxyl-terminal domain, which lies on the surface of the assembled nucleocapsid. A hinge is thought to exist at the junction of these two domains thereby imparting some flexibility to this protein (see Fields et al. (ed.), 1991, Fundamental Virology, 2nd ed., Raven Press, New York, incorporated by reference herein in its entirety). The matrix protein (M), is apparently involved with viral assembly and interacts with both the viral membrane as well as the nucleocapsid proteins. The phosphoprotein (P), which is subject to phosphorylation, is thought to play a regulatory role in transcription and can also be involved in methylation, phosphorylation and polyadenylation. The fusion glycoprotein (F) interacts with the viral membrane and is first produced as an inactive precursor then cleaved post-translationally to produce two disulfide linked polypeptides. The active F protein is also involved in penetration of the parainfluenza virion into host cells by facilitating fusion of the viral envelope with the host cell plasma membrane. Id. The glycoprotein, hemagglutinin-neuraminidase (HN), protrudes from the envelope allowing the virus to contain both hemagglutinin and neuraminidase activities. HN is strongly hydrophobic at its amino terminal which functions to anchor the HN protein into the lipid bilayer. Id. Finally, the large polymerase protein (L) plays an important role in both transcription and replication. Id.

Currently, treatment for PIV comprises treatment of specific symptoms. In most cases, rest, fluids, and a comfortable environment are sufficient therapy for PIV infection. In cases in which fever is high, acetaminophen is recommended over aspirin, especially in children to avoid the risk of Reye's syndrome with influenza. For croup associated with PIV infection, therapies such as humidified air, oxygen, aerosolized racemic epinephrine, and oral dexamethasone (a steroid) are recommended to decrease upper airway swelling and intravenous fluids are administered for dehydration. Therapy for bronchiolitis associated with PIV infection include supportive therapy (e.g., oxygen, humidified air, chest clapping, and postural drainage to remove secretions, rest, and clear fluids) and administration of albuterol or steroids. Antibiotic, anti-viral, and/or antifungal agents can be administered to prevent secondary respiratory infections. See Merck Manual of Diagnosis and Therapy (17th ed., 1999).

Respiratory syncytial virus (“RSV”) is the leading cause of serious lower respiratory tract disease in infants and children (Feigen et al., eds., 1987, Textbook of Pediatric Infectious Diseases, WB Saunders, Philadelphia at pages 1653-1675; New Vaccine Development, Establishing Priorities, Vol. 1, 1985, National Academy Press, Washington D.C. at pages 397-409; and Ruuskanen et al., 1993, Curr. Probl. Pediatr. 23:50-79). The yearly epidemic nature of RSV infection is evident worldwide, but the incidence and severity of RSV disease in a given season vary by region (Hall, C. B., 1993, Contemp. Pediatr. 10:92-110). In temperate regions of the northern hemisphere, it usually begins in late fall and ends in late spring. Primary RSV infection occurs most often in children from 6 weeks to 2 years of age and uncommonly in the first 4 weeks of life during nosocomial epidemics (Hall et al., 1979, New Engl. J. Med. 300:393-396). Children at increased risk from RSV infection include, but are not limited to, preterm infants (Hall et al, 1979, New Engl. J. Med. 300:393-396) and children with bronchopulmonary dysplasia (Groothuis et al., 1988, Pediatrics 82:199-203), congenital heart disease (MacDonald et al., New Engl. J. Med. 307:397-400), congenital or acquired immunodeficiency (Ogra et al., 1988, Pediatr. Infect. Dis. J. 7:246-249; and Pohl et al., 1992, J. Infect. Dis. 165:166-169), and cystic fibrosis (Abman et al., 1988, J. Pediatr. 113:826-830). The fatality rate in infants with heart or lung disease who are hospitalized with RSV infection is 3%-4% (Navas et al., 1992, J. Pediatr. 121:348-354).

RSV infects adults as well as infants and children. In healthy adults, RSV causes predominantly upper respiratory tract disease. It has recently become evident that some adults, especially the elderly, have symptomatic RSV infections more frequently than had been previously reported (Evans, A. S., eds., 1989, Viral Infections of Humans Epidemiology and Control, 3rd ed., Plenum Medical Book, New York at pages 525-544). Several epidemics also have been reported among nursing home patients and institutionalized young adults (Falsey, A. R., 1991, Infect. Control Hosp. Epidemiol. 12:602-608; and Garvie et al., 1980, Br. Med. J. 281:1253-1254). Finally, RSV can cause serious disease in immunosuppressed persons, particularly bone marrow transplant patients (Hertz et al., 1989, Medicine 68:269-281).

Therapies available for the treatment of established RSV disease are limited. Severe RSV disease of the lower respiratory tract often requires considerable supportive care, including administration of humidified oxygen and respiratory assistance (Fields et al., eds, 1990, Fields Virology, 2nd ed., Vol. 1, Raven Press, New York at pages 1045-1072).

While a vaccine might prevent RSV infection, no vaccine is yet licensed for this indication. A major obstacle to vaccine development is safety. A formalin-inactivated vaccine, though immunogenic, unexpectedly caused a higher and more severe incidence of lower respiratory tract disease due to RSV in immunized infants than in infants immunized with a similarly prepared trivalent parainfluenza vaccine (Kim et al., 1969, Am. J. Epidemiol. 89:422-434; and Kapikian et al, 1969, Am. J. Epidemiol. 89:405-421). Several candidate RSV vaccines have been abandoned and others are under development (Murphy et al., 1994, Virus Res. 32:13-36), but even if safety issues are resolved, vaccine efficacy must also be improved. A number of problems remain to be solved. Immunization would be required in the immediate neonatal period since the peak incidence of lower respiratory tract disease occurs at 2-5 months of age. The immaturity of the neonatal immune response together with high titers of maternally acquired RSV antibody can be expected to reduce vaccine immunogenicity in the neonatal period (Murphy et al., 1988, J. Virol. 62:3907-3910; and Murphy et al., 1991, Vaccine 9:185-189). Finally, primary RSV infection and disease do not protect well against subsequent RSV disease (Henderson et al., 1979, New Engl. J. Med. 300:530-534).

Currently, the only approved approach to prophylaxis of RSV disease is passive immunization. Initial evidence suggesting a protective role for IgG was obtained from observations involving maternal antibody in ferrets (Prince, G. A., Ph.D. diss., University of California, Los Angeles, 1975) and humans (Lambrecht et al., 1976, J. Infect. Dis. 134:211-217; and Glezen et al., 1981, J. Pediatr. 98:708-715). Hemming et al. (Morell et al., eds., 1986, Clinical Use of Intravenous Immunoglobulins, Academic Press, London at pages 285-294) recognized the possible utility of RSV antibody in treatment or prevention of RSV infection during studies involving the pharmacokinetics of an intravenous immune globulin (IVIG) in newborns suspected of having neonatal sepsis. They noted that one infant, whose respiratory secretions yielded RSV, recovered rapidly after IVIG infusion. Subsequent analysis of the IVIG lot revealed an unusually high titer of RSV neutralizing antibody. This same group of investigators then examined the ability of hyperimmune serum or immune globulin, enriched for RSV neutralizing antibody, to protect cotton rats and primates against RSV infection (Prince et al., 1985, Virus Res. 3:193-206; Prince et al., 1990, J. Virol. 64:3091-3092; Hemming et al., 1985, J. Infect. Dis. 152:1083-1087; Prince et al., 1983, Infect. Immun. 42:81-87; and Prince et al, 1985, J. Virol. 55:517-520). Results of these studies suggested that RSV neutralizing antibody given prophylactically inhibited respiratory tract replication of RSV in cotton rats. When given therapeutically, RSV antibody reduced pulmonary viral replication both in cotton rats and in a nonhuman primate model. Furthermore, passive infusion of immune serum or immune globulin did not produce enhanced pulmonary pathology in cotton rats subsequently challenged with RSV.

Recent clinical studies have demonstrated the ability of this passively administered RSV hyperimmune globulin (RSV IVIG) to protect at-risk children from severe lower respiratory infection by RSV (Groothius et al., 1993, New Engl. J. Med. 329:1524-1530; and The PREVENT Study Group, 1997, Pediatrics 99:93-99). While this is a major advance in preventing RSV infection, this therapy poses certain limitations in its widespread use. First, RSV IVIG must be infused intravenously over several hours to achieve an effective dose. Second, the concentrations of active material in hyperimmune globulins are insufficient to treat adults at risk or most children with comprised cardiopulmonary function. Third, intravenous infusion necessitates monthly hospital visits during the RSV season. Finally, it can prove difficult to select sufficient donors to produce a hyperimmune globulin for RSV to meet the demand for this product. Currently, only approximately 8% of normal donors have RSV neutralizing antibody titers high enough to qualify for the production of hyperimmune globulin.

Two glycoproteins, F and G, on the surface of RSV have been shown to be targets of neutralizing antibodies (Fields et al., 1990, supra; and Murphy et al., 1994, supra). These two proteins are also primarily responsible for viral recognition and entry into target cells; G protein binds to a specific cellular receptor and the F protein promotes fusion of the virus with the cell. The F protein is also expressed on the surface of infected cells and is responsible for subsequent fusion with other cells leading to syncytia formation. Thus, antibodies to the F protein can directly neutralize virus or block entry of the virus into the cell or prevent syncytia formation. Although antigenic and structural differences between A and B subtypes have been described for both the G and F proteins, the more significant antigenic differences reside on the G glycoprotein, where amino acid sequences are only 53% homologous and antigenic relatedness is 5% (Walsh et al., 1987, J. Infect. Dis. 155:1198-1204; and Johnson et al., 1987, Proc. Natl. Acad. Sci. USA 84:5625-5629). Conversely, antibodies raised to the F protein show a high degree of cross-reactivity among subtype A and B viruses. Comparison of biological and biochemical properties of 18 different murine MAbs directed to the RSV F protein resulted in the identification of three distinct antigenic sites that are designated A, B, and C. (Beeler and Coelingh, 1989, J. Virol. 7:2941-2950). Neutralization studies were performed against a panel of RSV strains isolated from 1956 to 1985 that demonstrated that epitopes within antigenic sites A and C are highly conserved, while the epitopes of antigenic site B are variable.

A humanized antibody directed to an epitope in the A antigenic site of the F protein of RSV, palivizumab, is approved for intramuscular administration to pediatric patients for prevention of serious lower respiratory tract disease caused by RSV at recommended monthly doses of 15 mg/kg of body weight throughout the RSV season (November through April in the northern hemisphere). Palivizumab is a composite of human (95%) and murine (5%) antibody sequences. See, Johnson et al., 1997, J. Infect. Diseases 176:1215-1224 and U.S. Pat. No. 5,824,307, the entire contents of which are incorporated herein by reference. The human heavy chain sequence was derived from the constant domains of human IgG₁ and the variable framework regions of the VH genes of Cor (Press et al., 1970, Biochem. J. 117:641-660) and Cess (Takashi et al., 1984, Proc. Natl. Acad. Sci. USA 81:194-198). The human light chain sequence was derived from the constant domain of C.kappa. and the variable framework regions of the VL gene K104 with J.kappa.-4 (Bentley et al., 1980, Nature 288:5194-5198). The murine sequences derived from a murine monoclonal antibody, Mab 1129 (Beeler et al., 1989, J. Virology 63:2941-2950), in a process which involved the grafting of the murine complementarity determining regions into the human antibody frameworks.

Recently, a new member of the Paramyxoviridae family has been isolated from 28 children with clinical symptoms reminiscent of those caused by human respiratory syncytial virus (“hRSV”) infection, ranging from mild upper respiratory tract disease to severe bronchiolitis and pneumonia (Van Den Hoogen et al, 2001, Nature Medicine 7:719-724). The new virus was named human metapneumovirus (hMPV) based on sequence homology and gene constellation. The study further showed that by the age of five years virtually all children in the Netherlands have been exposed to hMPV and that the virus has been circulating in humans for at least half a century.

The genomic organization of human metapneumovirus is described in van den Hoogen et al., 2002, Virology 295:119-132. Human metapneumovirus has recently been isolated from patients in North America (Peret et al., 2002, J. Infect. Diseases 185:1660-1663).

Human metapneumovirus is related to avian metapneumovirus. For example, the F protein of hMPV is highly homologous to the F protein of avian pneumovirus (“APV”). Alignment of the human metapneumoviral F protein with the F protein of an avian pneumovirus isolated from Mallard Duck shows 85.6% identity in the ectodomain. Alignment of the human metapneumoviral F protein with the F protein of an avian pneumovirus isolated from Turkey (subgroup B) shows 75% identity in the ectodomain. See, e.g., co-owned and co-pending Provisional Application No. 60/358,934, entitled “Recombinant Parainfluenza Virus Expression Systems and Vaccines Comprising Heterologous Antigens Derived from Metapneumovirus,” filed on Feb. 21, 2002, by Haller and Tang, which is incorporated herein by reference in its entirety.

Respiratory disease caused by an APV was first described in South Africa in the late 1970s (Buys et al., 1980, Turkey 28:36-46) where it had a devastating effect on the turkey industry. The disease in turkeys was characterized by sinusitis and rhinitis and was called turkey rhinotracheitis (TRT). The European isolates of APV have also been strongly implicated as factors in swollen head syndrome (SHS) in chickens (O'Brien, 1985, Vet. Rec. 117:619-620). Originally, the disease appeared in broiler chicken flocks infected with Newcastle disease virus (NDV) and was assumed to be a secondary problem associated with Newcastle disease (ND). Antibody against European APV was detected in affected chickens after the onset of SHS (Cook et al., 1988, Avian Pathol. 17:403-410), thus implicating APV as the cause.

The avian pneumovirus is a single stranded, non-segmented RNA virus that belongs to the sub-family Pneumovirinae of the family Paramyxoviridae, genus metapneumovirus (Cavanagh and Barrett, 1988, Virus Res. 11:241-256; Ling et al., 1992, J. Gen. Virol. 73:1709-1715; Yu et al., 1992, J. Gen. Virol. 73:1355-1363). The Paramyxoviridae family is divided into two sub-families: the Paramyxovirinae and Pneumovirinae. The subfamily Paramyxovirinae includes, but is not limited to, the genera: Paramyxovirus, Rubulavirus, and Morbillivirus. Recently, the sub-family Pneumovirinae was divided into two genera based on gene order, i.e., pneumovirus and metapneumovirus (Naylor et al., 1998, J. Gen. Virol., 79:1393-1398; Pringle, 1998, Arch. Virol. 143:1449-1159). The pneumovirus genus includes, but is not limited to, human respiratory syncytial virus (hRSV), bovine respiratory syncytial virus (bRSV), ovine respiratory syncytial virus, and mouse pneumovirus. The metapneumovirus genus includes, but is not limited to, European avian pneumovirus (subgroups A and B), which is distinguished from hRSV, the type species for the genus pneumovirus (Naylor et al., 1998, J. Gen. Virol., 79:1393-1398; Pringle, 1998, Arch. Virol. 143:1449-1159). The US isolate of APV represents a third subgroup (subgroup C) within metapneumovirus genus because it has been found to be antigenically and genetically different from European isolates (Seal, 1998, Virus Res. 58:45-52; Senne et al., 1998, In: Proc. 47th WPDC, California, pp. 67-68).

Electron microscopic examination of negatively stained APV reveals pleomorphic, sometimes spherical, virions ranging from 80 to 200 nm in diameter with long filaments ranging from 1000 to 2000 nm in length (Collins and Gough, 1988, J. Gen. Virol. 69:909-916). The envelope is made of a membrane studded with spikes 13 to 15 nm in length. The nucleocapsid is helical, 14 nm in diameter and has 7 nm pitch. The nucleocapsid diameter is smaller than that of the genera Paramyxovirus and Morbillivirus, which usually have diameters of about 18 nm.

Avian pneumovirus infection is an emerging disease in the USA despite its presence elsewhere in the world in poultry for many years. In Can 1996, a highly contagious respiratory disease of turkeys appeared in Colorado, and an APV was subsequently isolated at the National Veterinary Services Laboratory (NVSL) in Ames, Iowa (Senne et al, 1997, Proc. 134th Ann. Mtg., AVMA, pp. 190). Prior to this time, the United States and Canada were considered free of avian pneumovirus (Pearson et al., 1993, In: Newly Emerging and Re-emerging Avian Diseases: Applied Research and Practical Applications for Diagnosis and Control, pp. 78-83; Hecker and Myers, 1993, Vet. Rec. 132:172). Early in 1997, the presence of APV was detected serologically in turkeys in Minnesota. By the time the first confirmed diagnosis was made, APV infections had already spread to many farms. The disease is associated with clinical signs in the upper respiratory tract: foamy eyes, nasal discharge and swelling of the sinuses. It is exacerbated by secondary infections. Morbidity in infected birds can be as high as 100%. The mortality can range from 1 to 90% and is highest in six to twelve week old poults.

Avian pneumovirus is transmitted by contact. Nasal discharge, movement of affected birds, contaminated water, contaminated equipment; contaminated feed trucks and load-out activities can contribute to the transmission of the virus. Recovered turkeys are thought to be carriers. Because the virus is shown to infect the epithelium of the oviduct of laying turkeys and because APV has been detected in young poults, egg transmission is considered a possibility.

Based upon the recent work with hMPV, hMPV likewise appears to be a significant factor in human, particularly, juvenile respiratory disease.

Thus, theses three viruses, RSV, hMPV, and PIV, cause a significant portion of human respiratory disease. Accordingly, a broad spectrum therapy is needed to reduce the incidence of viral respiratory disease caused by these viruses.

Mycobacterium tuberculosis infects 1.9 billion and the active disease, tuberculosis (“TB”) results in 1.9 million deaths around the world each year. (Dye et al., 1999, JAMA 282:677-686). After a century of steadily declining rates of TB cases in the United States, the downward trend was reversed in the late 1980s as a result of the emergence of a multidrug-resistant strain of M. tuberculosis, the HIV epidemic, and influx of immigrants. (Navin et al., 2002, Emerg. Infect. Dis. 8:11).

M. tuberculosis is an obligate aerobe, nonmotile rod-shaped bacterium. In classic cases of tuberculosis, M. tuberculosis complexes are in the well-aerated upper lobes of the lungs. M. tuberculosis are classified as acid-fast bacteria due to the impermeability of the cell wall by certain dyes and stains. The cell wall of M. tuberculosis, composed of peptidoglycan and complex lipids, is responsible for the bacterium's resistance to many antibiotics, acidic and alkaline compounds, osmotic lysis, and lethal oxidations, and survival inside macrophages.

TB progresses in five stages. In the first stage, the subject inhales the droplet nuclei containing less than three bacilli. Although alveolar macrophages take up the M. tuberculosis, the macrophages are not activated and do not destroy the bacterium. Seven to 21 days after the initial infection, the M. tuberculosis multiples within the macrophages until the macrophages burst, which attracts additional macrophages to the site of infection that phagocytose the M. tuberculosis, but are not activated and thus do not destroy the M. tuberculosis. In stage 3, lymphocytes, particularly T-cells, are activated and cytokines, including IFN activate macrophages capable of destroying M. tuberculosis are produced. At this stage, the patient is tuberculin-positive and a cell mediated immune response, including activated macrophages releasing lytic enzymes and T cell secreting cytokines, is initiated. Although, some macrophages are activated against the M. tuberculosis, the bacteria continue to multiply within inactivated macrophages and begin to grow tubercles which are characterized by semi-solid centers. In stage 4, tubercles can invade the bronchus, other parts of the lung, and the blood supply line and the patient can exhibit secondary lesions in other parts of the body, including the genitourinary system, bones, joints, lymph nodes, and peritoneum. In the final stage, the tubercles liquefy inducing increased growth of M. tuberculosis. The large bacterium load causes the walls of nearby bronchi to rupture and form cavities that enables the infection to spread quickly to other parts of the lung.

Current therapies available for the treatment of TB comprise an initial two month regime of multiple antibiotics, such as rifampcin, isoniazid, pyranzinamide, ethambutol, or streptomycin. In the next four months, only rifampicin and isoniazid are administered to destroy persisting M. tuberculosis. Although proper prescription and patient compliance results in a cure in most cases, the number of deaths from TB has been on the rise as a result from the emergence of new M. tuberculosis strains resistant to current antibiotic therapies. (Rattan et al., 1998, Emerging Infectious Diseases, 4(2):195-206). In addition, fatal and severe liver injury has been associated with treatment of latent TB with rifampcin and pyranzinamide. (CDC Morbidity and Mortality Weekly Report, 51(44):998-999).

The number of systemic invasive fungal infections rose sharply in the past decade due to the increase in the at-risk patient population as a result of organ transplants, oncology, human immunodeficiency virus, use of vascular catheters, and misuse of broad spectrum antibiotics. Dodds et al, 2000 Pharmacotherapy 20(11): 1335-1355. Seventy percent of fungal-related deaths are caused by Candida species, Aspergillus species, and Cryptococcus neoformans. Yasuda, California Journal of Health-System Pharmacy, Can/June 2001, pp. 4-11.

Eighty percent of all major systemic fungal infections are due to Candida species. The Merk Manual of Diagnosis and Therapy, 17th ed., 1999. Invasive candidiasis is most often caused by Candida albicans, Candida troicalis, and Candida glabrata in immunosuppressd patients. Id. Candidiasis is a defining opportunistic infection of AIDS, infecting the esophagus, trachea, bronchi, and lungs. Id. In HIV-infected patients, candidiasis is usually mucocutaneous and infects the oropharynx, the esophagus, and the vagina. Ampel, April-June 1996, Emerg. Infect. Dis. 2(2): 109-116.

Candida species are commensals that colonize the normal GI tract and skin. The Merk Manual of Diagnosis and Therapy, Berkow et al (eds.), 17th ed., 1999. Thus, cultures of Candidia from sputum, the mouth, urine, stool, vagina, or skin does not necessarily indicate an invasive, progressive infection. Id. In most cases, diagnosis of candidiasis requires presentation of a characteristic clinical lesion, documentation of histopathologic evidence of tissue invasion, or the exclusion of other causes. Id. Symptoms of systemic candidiasis infection of the respiratory tract are typically nonspecific, including dysphagia, coughing, and fever. Id.

All forms of candidiasis are considered serious, progressive, and potentially fatal. Id. Therapies for the treatment of candidiasis typically include the administration of the combination of the anti-fungal agents amphotericin B and flucytosine. Id. Unfortunately, acute renal failure has been associated with amphotericin B therapy. Dodds, supra. Fluconazole is not as effective as amphotericin B in treating certain species of Candida, but is useful as initial therapy in high oral or intravenous doses while species identification is pending. The Merk Manual of Diagnosis and Therapy, 17th ed., 1999. Fluconazole, however, has led to increasing treatment failures and anti-fungal resistance. Ampel, supra. Thus, there is a need for novel therapies of systemic candidiasis.

Aspergillus includes 132 species and 18 variants among which Aspergillus fumigatus is involved in 80% of Aspergillus-related diseases. Kurp et al, 1999, Medscape General Medicine 1(3). Aspergillus fumigatus is the most common cause of invasive pulmonary aspergillosis that extends rapidly, causing progressive, and ultimately fatal respiratory failure. The Merk Manual of Diagnosis and Therapy, 17th ed., 1999. Patients undergoing long-term high-dose corticosteroid therapy, organ transplant patients, patients with hereditary disorders of neutrophil function, and patients infected with AIDS are at risk for aspergillosis.

Clinical manifestations of invasive pulmonary infection by Aspergillus include fever, cough, and chest pain. Aspergillus colonize preexisting cavity pulmonary lesions in the form of aspergilloma (fungus ball) which is composed of tangled masses hyphae, fibrin exudate, and inflammatory cells encapsulated by fibrous tissue. Id. Aspergillomas usually form and enlarge in pulmonary cavities originally caused by bronchiectasis, neoplasm, TB, and other chronic pulmonary infections. Id. Most aspergillomas do not respond to or require systemic anti-fungal therapy. Id. However, invasive infections often progress rapidly and are fatal, thus aggressive therapy comprising IV amphotericin B or oral itraconazole is required. Id. Unfortunately, high-dose amphotericin B can cause renal failure and itraconazole is effective only in moderately severe cases. Id. Therefore, there is a need for new therapies for the treatment of aspergillosis.

Cases of cryptococcosis were rare before the HIV epidemic. Ampel, supra. AIDS patients, patients with Hodgkin's or other lymphomas or sarcoidosis, and patients undergoing long-term corticosteroid therapy are at increased risk for cryptococcosis. The Merk Manual of Diagnosis and Therapy, 17th ed., 1999. In most cases, cryptococcal infections are self-limited, but AIDS-associated cryptococcal infection can be in the form of a severe, progressive pneumonia with acute dyspnea and primary lesions in the lungs. Id. In cases of progressive disseminated cryptococcosis affecting non-immunocompromised patients, chronic meningitis is most common without clinically evident pulmonary lesions. Id.

Immunocompetent patients do not always require the administration of a therapy to treat localized pulmonary cryptococcosis. However, when such patients are administered a therapy for the treatment of localized pulmonary cryptococcosis, it typically consists of administration of amphotericin B with or without flucytosine. Id. AIDS patients are generally administered an initial therapy consisting of amphotericin B and flucytosine and then oral fluconazole thereafter to treat cryptococcosis. Id. Renal and hematologic function of all patients receiving amphotericin B with or without flucytosine must be evaluated before and during therapy since flucytosine blood levels must be monitored to limit toxicity and administration of flucytosine can not be safe for patients with preexisting renal failure or bone marrow dysfunction. Id. Thus, new therapies for the treatment of cryptococcosis are needed.

### Peroxisome Proliferator Activated Receptors (PPARs)

Peroxisome Proliferator Activated Receptors (PPARs) are orphan receptors belonging to the steroid/retinoid receptor superfamily of ligand-activated transcription factors. See, for example Willson T. M. and Wahli, W., Curr. Opin. Chem. Biol. (1997) Vol 1 pp 235 241 and Willson T. M. et. al., J. Med. Chem. (2000) Vol 43 p527 549. The binding of agonist ligands to the receptor results in changes in the expression level of mRNA's encoded by PPAR target genes.

Three mammalian Peroxisome Proliferator-Activated Receptors have been isolated and termed PPAR-alpha (PPARα), PPAR-gamma (PPARγ) and PPAR-delta (PPARδ) also known as NUC1 or PPAR-beta (PPARβ)). These PPARs regulate expression of target genes by binding to DNA sequence elements, termed PPAR response elements (PPRE) (Berger, J. et al., The Journal of Biological Chemistry, 1999, 274 (10), 6718-6725). To date, PPRE's have been identified in the enhancers of a number of genes encoding proteins that regulate lipid metabolism suggesting that PPARs play a pivotal role in the adipogenic signaling cascade and lipid homeostasis (H. Keller and W. Wahli, Trends Endocrin. Met 291 296, 4 (1993)).

PPAR-alpha is mainly expressed in the liver, heart, intestinal tract, kidney and macrophage, and, after being activated, can increase the metabolism of fatty acids, alleviate the inflammatory response in macrophages, and reduce low density lipoprotein cholesterol; PPAR-gamma is expressed in the adipocyte, placentoma and other tissues, and, after being activated, can not only lower the blood glucose level and increase the insulin sensitivity, but also play a key role in lipid metabolism, cytokine antagonization, anti-inflammation, immuno regulation and blood pressure regulation, etc. (Kasuga, J. et al., Bioorg. Med. Chem. 2007, 15, 5177-5190). In contrast to the other two subtypes, the physiologic function of PPAR-delta is not clear up to now. However, it has been shown in recent studies on animal models for pharmacology experiments that, the PPAR-delta can increase the fatty acid catabiosis and energy uncoupling in adipose tissue and muscle, and can suppress the macrophage-originated inflammation. Due to various functions in controlling gaining weight of human body, enhancing body's durability, increasing the insulin sensitivity and improving artherosclerosis, the ligands for the PPAR-delta can be an effective medicament for the treatment of hyperlipidemia, obesity, insulin resistance, and artherosclerosis.

In some embodiments, the PPAR is PPARδ or PPARγ.

### PPAR Agonists

As used herein, the term “PPAR agonist” refers to a compound/composition which achieves at least 10% (e.g., 10%, 15%, 20%, 25%, 30%, 35%, 40%, 45%, 50%, 55%, 60%, 65%, 70%, 75%, 80%, 85%, 90%, 95% or more) activation of a PPAR, e.g., PPARδ or PPARγ. In some embodiments, the PPAR agonist is a selective PPARδ agonist. As used herein, a “selective PPARδ agonist” is a PPARδ agonist whose EC₅₀ for PPARδ is at least 10 fold lower than its EC₅₀ for any of the other PPARs, such as PPARγ and PPARα. Such selective compounds can be referred to as “10-fold selective.” EC₅₀ is defined in the transfection assay described, for example in WO 00/08002 and is the concentration at which a compound achieves 50% of its maximum activity. In some embodiments, the PPARδ agonist is greater than 100-fold selective PPARδ agonist.

In some embodiments, the PPAR agonist is a selective PPARγ agonist. As used herein, a “selective PPARγ agonist” is a PPARγ agonist whose EC₅₀ for PPARγ is at least 10 fold lower than its EC₅₀ for any of the other PPARs, such as PPARδ and PPARα. Such selective compounds can be referred to as “10-fold selective.” As discussed above, EC₅₀ is defined in the transfection assay described, for example in WO 00/08002 and is the concentration at which a compound achieves 50% of its maximum activity. In some embodiments, the PPARγ agonist is greater than 100-fold selective PPARγ agonist

Without limitations, a PPAR agonist can be selected from the group consisting of small organic or inorganic molecules; saccharines; oligosaccharides; polysaccharides; biological macromolecules; peptides; proteins; peptide analogs and derivatives; peptidomimetics; antibodies; antigen binding fragments of antibodies; nucleic acids, e.g., oligonucleotides, antisense oligonucleotides, siRNAs, shRNAs, ribozymes, aptamers, microRNAs, pre-microRNAs, plasmid DNA, activating RNAs, etc. . . . ; nucleic acid analogs and derivatives; an extract made from biological materials such as bacteria, plants, fungi, or animal cells; animal tissues; naturally occurring or synthetic compositions; and any combinations thereof. It is noted that the term “PPARδ agonist” as used herein comprises naturally occurring botanical sources that include PPARδ activator compounds or components.

In some embodiments, the PPAR agonist, e.g., PPARδ or PPARγ agonist is a thiazole derivative. In some embodiments, the PPAR agonist, e.g., PPARδ or PPARγ PPARδ agonist is an oxazole derivative. In some embodiments, the PPAR agonist, e.g., PPARδ or PPARγ PPARδ agonist is thiazolidinedione.

In some embodiments, the PPAR agonist, e.g., PPARδ or PPARγ agonist is a compound of formula (I):

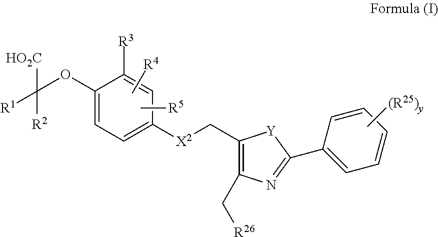

wherein:
R¹ and R² are independently hydrogen or C₁₋₃alkyl;
X² is O, S, or CH₂;
R³, R⁴, and R⁵ are independently H, C₁₋₃alkyl, OCH₃, CF₃, OCF₃, CN, allyl, or halogen;
Y is S or O;
each R²⁵ is independently CH₃, OCH₃, CF₃, or halogen;
each R²⁶ is independently for each occurrence

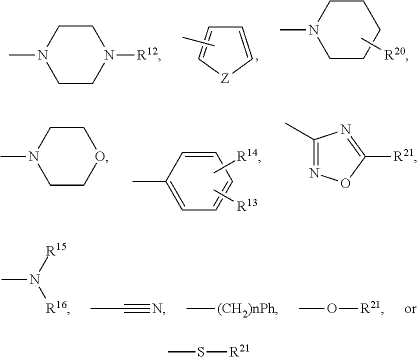

R¹² is independently for each occurrence C₁₋₆alkyl, C₁₋₆alkylenearyl,

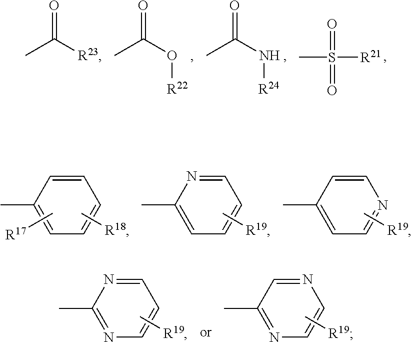

R¹³ and R¹⁴ are independently hydrogen, halogen, CN, perfluoroC₁₋₆alkyl, perfluoro-O—C₁₋₆alkyl, C₁₋₆alkyl, —OC₁₋₆alkyl, —C₁₋₆alkyleneOC₁₋₆alkyl, —SC₁₋₆alkyl, or aryl;
R¹⁵ and R¹⁶ are independently hydrogen, C₁₋₆alkyl, C₃₋₆cycloalkyl optionally substituted with 1 or 2 C₁₋₃alkyl groups, or R¹² as defined above;
R¹⁷ and R¹⁸ are independently hydrogen, halogen, hydroxy, —CN, C₁₋₆alkyl, C₁₋₆ perfluoroalkyl, C₁₋₆acyl, —OC₁₋₆alkyl, perfluoroOC₁₋₆alkyl, or C₁₋₆hydroxyalkyl;
R¹⁹ is independently for each occurrence hydrogen or C₁₋₆alkyl;
R²⁰ is independently for each occurrence C₁₋₆alkyl, aryl, —OC₁₋₆alkyl, hydroxy, C₁₋₆ hydroxyalkyl, or 1-alkoxyC₁₋₆alkyl;
R²¹ is independently for each occurrence C₁₋₆alkyl, —C₁₋₆alkylenearyl, aryl, or aryl-heteroaryl;
R²² is independently for each occurrence independently for each occurrence C₁₋₆alkyl, aryl, or —C₁₋₆alkylenearyl;
R²³ is C₁₋₆alkyl, C₃₋₆cycloalkyl, or aryl;
R²⁴ is independently for each occurrence C₁₋₆alkyl, C₁₋₆alkylenearyl, C₃₋₆cycloalkyl, or aryl;
Z is independently for each occurrence O, N, or S (note that when Z is N, the depicted bond can be attached to the nitrogen in the ring as well as any of the carbons in the ring);
y is 0, 1, 2, 3, 4, or 5; and
n is 1, 2, or 3.

Compounds of formula (I) can be synthesized by, for example, the method described in U.S. Pat. No. 7,084,161, content of which is incorporated herein by reference in its entirety. In some embodiments, compounds of formula (I) that can be used in the present invention are described in U.S. Pat. No. 7,229,998 and No. 7,0084,161; and U.S. Patent Application Publication No. 2007/0225294 and No. 2005/0131035, content of all of which is incorporated herein by reference in their entirety.

In some embodiments, a PPAR agonist, e.g., a PPARδ or PPARγ agonist of the invention is:

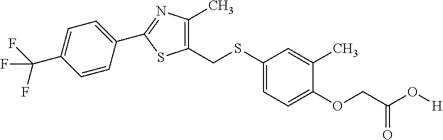 [SMILES: Cc1cc(SCc2sc(-c3ccc(C(F)(F)F)cc3)nc2C)ccc1OCC(=O)O]

((2-methyl-4-(((4-methyl-2-(4-(trifluoromethyl)phenyl)-5-(thiazolyl)methyl)thio)phenoxy)acetic acid (GW501516)).

Exemplary PPARδ agonists, e.g., PPARδ or PPARγ agonists are described in, for example, WO1997/028149, WO1997/028149, WO1999/062872, WO2000/031055, WO2000/073252, WO2001/000603, WO2001/000603, WO2001/036401, WO2001/079197, WO2001/079197, WO2002/014291, WO2002/046154, WO2002/046176, WO2002/050048, WO2002/053547, WO2002/076957, WO2002/076959, WO2002/100813, WO2003/016291, WO2003/018010, WO2003/033493, WO2003/074495, WO2003/099793, WO2003/099793, WO2004/022551, WO2004/058174, WO2004/063165, WO2004/063184, WO2004/092117, WO2004/111020, WO2005/016335, WO2005/030694, WO2005/037763, WO2005/049606, WO2005/060958, WO2005/085235, WO2005/090966, WO2005/097763, WO2005/097786, WO2005/105726, WO2005/105736, WO2005/105754, WO2005/113600, WO2006/031969, WO2006/041197, WO2006/055187, WO2006/059744, WO2006/084176, WO2006/090920, JP2003-171275A, JP2005-179281A, content of all of which is incorporated herein by reference in their entirety.

Some specific examples of the PPAR agonists amenable to the present incention include, but are not limited to, 1-methylethyl 2-(4-(4-chlorobenzoyl)phenoxy)-2-methylpropionate (fenofibrate), ((4-chloro-6-((2,3-dimethylphenyl)amino)-2-pyrimidinyl)thio)acetic acid (WY-14643), (4-(3-(4-acetyl-3-hydroxy-2-propyl)phenoxy)propoxyphenoxy)acetic acid (L-165041), (2-methyl-4-(((4-methyl-2-(4-(trifluoromethyl)phenyl)-5-(thiazolyl)methyl)thio)phenoxy)acetic acid (GW501516), (4-4(2-(3-fluoro-4-(trifluoromethyl)phenyl)-4-methyl-5-thiazolyl)methyl)thio)-2-methylphenoxy)acetic acid (GW-0742), 2-methyl-4-((2R)-2-(3-methyl-5-(4-(trifluoromethyl)phenyl)-2-thienyl)propoxy)-benzenepropionic acid, 2-ethyl-2-(4-(4-(4-(4-methoxyphenyl)-piperazin-1-yl)-2-(4-trifluoromethyl-phenyl)-thiazol-5-ylmethylsulfanyl)-phenoxy)butyric acid (GSK-677954), (4-(3-(3-phenyl-7-propyl-benzofuran-6-yloxy)-propylsulfanyl)-phenyl)acetic acid (L-796449), 2-(4-(3-(1-(2-(2-chloro-6-fluoro-phenyl)-ethyl)-3-(2,3-dichloro-phenyl)-ureido)-propyl)-phenoxy)-2-methylpropionic acid (GW-2433), 2-{2-methyl-4-[({4-methyl-2-[4-(trifluoromethyl)phenyl]-1,3-thiazol-5-yl}methyl)sulfanyl]phenoxy}acetic acid, 2-{2-methyf-4-[({4-methyl-2-[4-(trifluoromethyl)phenyl]-1,3-oxazol-5-yl}methyl)sulfanyl]phenoxy}acetic acid, methyl 2-{4-[({4-methyl-2-[4-(trifluoromethyl)phenyl]-1,3-thiazol-5-yl}methyl)sulfanyl]phenoxy}acetate, 2-{4-[({4-methyl-2-[4-(trifluoromethyl)phenyl]-1,3-thiazol-5-yl}methyl)sulfanyl]phenoxy}acetic acid, (E)-3-[2-methyl-4-({4-methyl-2-[4-(trifluoromethyl)phenyl]-1,3-thiazol-5-yl}methoxy)phenyl]-2-propenoic acid, 2-{3-chloro-4-[({4-methyl-2-[4-(trifluoromethyl)phenyl]-1,3-thiazol-5-yl}methyl)sulfanyl]phenyl}acetic acid, and metabolites of arachidonic acid, e.g., 4-hydroxy-2-nonenal (4-HNE), 4-hydroxydodeca-(2E,6Z)-dienal (4-HDDE), rosiglitazone, pioglitazone, troglitazone, netoglitazone (also known as MCC-555 or isaglitazone or neoglitazone), 5-BTZD, farglitazar, 677954 (GlaxoSmithKline), PLX204 (Plexxikon), LY 519818, L-783483, L-165461, L-16504, and the like.

It will also be appreciated by those skilled in the art that the PPAR agonists can also be utilized in the form of a pharmaceutically acceptable salt or solvate thereof. The physiologically acceptable salts include conventional salts formed from pharmaceutically acceptable inorganic or organic acids or bases as well as quaternary ammonium acid addition salts. More specific examples of suitable acid salts include hydrochloric, hydrobromic, sulfuric, phosphoric, nitric, perchloric, fumaric, acetic, propionic, succinic, glycolic, formic, lactic, maleic, tartaric, citric, palmoic, malonic, hydroxymaleic, phenylacetic, glutamic, benzoic, salicylic, fumaric, toluenesulfonic, methanesulfonic, naphthalene-2-sulfonic, benzenesulfonic hydroxynaphthoic, hydroiodic, malic, steroic, tannic and the like. Other acids such as oxalic, while not in themselves pharmaceutically acceptable, can be useful in the preparation of salts useful as intermediates in obtaining the compounds of the invention and their pharmaceutically acceptable salts. More specific examples of suitable basic salts include sodium, lithium, potassium, magnesium, aluminium, calcium, zinc, N,N′-dibenzylethylenediamine, chloroprocaine, choline, diethanolamine, ethylenediamine, N-methylglucamine and procaine salts. References herein to a PPAR agonist include both compounds and their pharmaceutically acceptable salts and solvates.

The present invention is directed to therapies which involve administering one or more PPAR agonist and compositions comprising said agonists to a subject, preferably a human subject, for preventing, treating, managing, or ameliorating an infection, a respiratory condition. In one embodiment, the invention provides a method of preventing, treating, managing, or ameliorating a respiratory disorder, said method comprising administering to a subject in need thereof a dose of a prophylactically or therapeutically effective amount of one or more PPAR agonists. In another embodiment, the invention provides a method of preventing, treating, managing, or ameliorating a respiratory infection, said method comprising administering a prophylactically or therapeutic effective amount of one or more PPAR agonists.

Agents Useful in Combination with PPAR Agonists

Embodiments of the method disclosed herein comprise administering to a subject in need thereof a PPAR agonist in combination with one or more therapies (e.g., one or more prophylactic or therapeutic agents). The one or more therapies can be administering a second therapeutic agent, i.e., a therapeutic or prophylactic agent. Without limitations, the second therapeutic agent can be another PPAR agonist or a molecule/composition other than a PPAR agonist. The present invention also provides compositions comprising one or more PPAR agonist and one or more prophylactic or therapeutic agents other than PPAR agonists and methods of preventing, managing, treating, or ameliorating an infection, a respiratory condition utilizing said compositions.

The term “co-administering,” “co-administration,” or “co-administer” refers to the administration of a PPAR agonist and a second therapy to the subject, wherein the PPAR agonist and the second therapy can be administered simultaneously, or at different times, as long as they work additively or synergistically.

When the second therapy is a therapeutic agent, the PPAR agonist and the therapeutic agent can be administered in the same formulation or in separate formulations. When administered in separate formulations, the PPAR agonist and the therapeutic agent can be administered within any time of each other. For example, the compounds can be administered within 24 hours, 12 hours, 6 hours, 5 hours, 4 hours, 3 hours, 2 hours, 1 hours, 45 minutes, 30 minute, 25 minutes, 20 minutes, 15 minutes, 10 minutes, 5 minutes or less of each other. When administered in separate formulations, either compound can be administered first. In some embodiments, the PPAR agonist is administered first.

Additionally, co-administration does not require the two compounds to be administered by the same route, i.e., the components of the combination therapy can be administered to a subject by the same or different routes of administration. As such, each can be administered independently or as a common dosage form. Further, the two compounds can be administered in any ratio to each other by weight or moles. For example, two compounds can be administered in a ratio of from about 50:1, 40:1, 30:1, 25:1, 20:1, 15:1, 10:1, 5:1, 3:1, 2:1, 1:1.75, 1.5:1, or 1.25:1 to 1:1.25, 1:1.5, 1.75, 1:2, 1:3, 1:4, 1:5, 1:10, 1:15, 1:20, 1:20, 1:30, 1:40, or 1:50. Their ratio can be based on the effective amount of either compound.

In some embodiments, the combination therapies comprise one or more PPAR agonist and at least one other therapy (e.g., at least one other prophylactic or therapeutic agent) which has a different mechanism of action than said agonists. In another embodiment, the combination therapies comprise one or more PPAR agonist and at least one other therapy (e.g., at least one other prophylactic agent) which has the same mechanism of action as said agonists. In certain embodiments, the combination therapies improve the prophylactic or therapeutic effect(s) of one or more agonists by functioning together with the agonists to have an additive or synergistic effect. In certain embodiments, the combination therapies reduce the side effects associated with the prophylactic or therapeutic agents.

Therapeutic or prophylactic agents include, but are not limited to, small molecules, synthetic drugs, peptides, polypeptides, proteins, nucleic acids (e.g., DNA and RNA nucleotides including, but not limited to, antisense nucleotide sequences, triple helices, RNAi, and nucleotide sequences encoding biologically active proteins, polypeptides or peptides) antibodies, synthetic or natural inorganic molecules, mimetic agents, and synthetic or natural organic molecules. Without wishing to be bound by a theory, the combination of PPAR agonist and the second therapy can act synergistically. Similarly, combination of different PPAR agonists can also act synergistically.

Exemplary therapeutic or prophylactic agents include, but are not limited to, those found in Harrison's Principles of Internal Medicine, 13^{th} Edition, Eds. T. R. Harrison et al. McGraw-Hill N.Y., NY; Physicians' Desk Reference, 50^{th} Edition, 1997, Oradell New Jersey, Medical Economics Co.; Pharmacological Basis of Therapeutics, 8^{th} Edition, Goodman and Gilman, 1990; United States Pharmacopeia, The National Formulary, USP XII NF XVII, 1990; current edition of Goodman and Oilman's The Pharmacological Basis of Therapeutics; and current edition of The Merck Index, the complete contents of all of which are incorporated herein by reference.

Without limitations, any therapy which is known to be useful, or which has been used or is currently being used for the prevention, management, treatment, or amelioration of a respiratory condition can be used in combination with a PPAR agonist in accordance with the methods disclosed herein, i.e., co-administered with the PPAR. See, e.g., Gilman et al., Goodman and Gilman's: The Pharmacological Basis of Therapeutics, 10th ed., McGraw-Hill, New York, 2001; The Merck Manual of Diagnosis and Therapy, Berkow, M. D. et al. (eds.), 17th Ed., Merck Sharp & Dohme Research Laboratories, Rahway, N.J., 1999; Cecil Textbook of Medicine, 20th Ed., Bennett and Plum (eds.), W.B. Saunders, Philadelphia, 1996 for information regarding therapies (e.g., prophylactic or therapeutic agents) which have been or are currently being used for preventing, treating, managing, or ameliorating an infection, a respiratory condition or one or more symptoms thereof. Examples of such agents include, but are not limited to, immunomodulatory agents, anti-inflammatory agents (e.g., adrenocorticoids, cortico steroids (e.g., beclomethasone, budesonide, flunisolide, fluticasone, triamcinolone, methlyprednisolone, prednisolone, prednisone, hydrocortisone), glucocorticoids, steroids, non-steriodal anti-inflammatory drugs (e.g., aspirin, ibuprofen, diclofenac, and COX-2 inhibitors), pain relievers, leukotreine antagonists (e.g., montelukast, methyl xanthines, zafirlukast, and zileuton), beta2-agonists (e.g., albuterol, biterol, fenoterol, isoetharie, metaproterenol, pirbuterol, salbutamol, terbutalin formoterol, salmeterol, and salbutamol terbutaline), anticholinergic agents (e.g., ipratropium bromide and oxitropium bromide), sulphasalazine, penicillamine, dapsone, antihistamines, anti-malarial agents (e.g., hydroxychloroquine), anti-viral agents, and antibiotics (e.g., dactinomycin (formerly actinomycin), bleomycin, erythomycin, penicillin, mithramycin, and anthramycin (AMC)).

### Immunomodulatory Agents

Any immunomodulatory agent well-known to one of skill in the art can be used in the methods and compositions of the invention. Immunomodulatory agents can affect one or more or all aspects of the immune response in a subject. Aspects of the immune response include, but are not limited to, the inflammatory response, the complement cascade, leukocyte and lymphocyte differentiation, proliferation, and/or effector function, monocyte and/or basophil counts, and the cellular communication among cells of the immune system. In some embodiments of the invention, an immunomodulatory agent modulates one aspect of the immune response. In some embodiments, an immunomodulatory agent modulates more than one aspect of the immune response. In some embodiments of the invention, the administration of an immunomodulatory agent to a subject inhibits or reduces one or more aspects of the subject's immune response capabilities. In some embodiments of the invention, the immunomodulatory agent enhances one or more aspects of a subject's immune response. In accordance with the invention, an immunomodulatory agent is not an PPARδ agonist. In certain embodiments, an immunomodulatory agent is not an anti-inflammatory agent. In some embodiments, an immunomodulatory agent is a chemotherapeutic agent. In yet other embodiments, an immunomodulatory agent is an agent other than a chemotherapeutic agent.

Examples of immunomodulatory agents include, but are not limited to, proteinaceous agents such as cytokines, peptide mimetics, and antibodies (e.g., human, humanized, chimeric, monoclonal, polyclonal, Fvs, ScFvs, Fab or F(ab)₂ fragments or epitope binding fragments), nucleic acid molecules (e.g., antisense nucleic acid molecules and triple helices), small molecules, organic compounds, and inorganic compounds. In particular, immunomodulatory agents include, but are not limited to, methotrexate, leflunomide, cyclophosphamide, cytoxan, Immuran, cyclosporine A, minocycline, azathioprine, antibiotics (e.g., FK506 (tacrolimus)), methylprednisolone (MP), corticosteroids, steroids, mycophenolate mofetil, rapamycin (sirolimus), mizoribine, deoxyspergualin, brequinar, malononitriloamindes (e.g., leflunamide), T cell receptor modulators, cytokine receptor modulators, and modulators mast cell modulators.

Examples of T cell receptor modulators include, but are not limited to, anti-T cell receptor antibodies (e.g., anti-CD4 antibodies (e.g., cM-T412 (Boeringer), IDEC-CE9.1.R™ (IDEC and SKB), mAB 4162W94, Orthoclone and OKTcdr4a (Janssen-Cilag)), anti-CD3 antibodies (e.g., Nuvion (Product Design Labs), OKT3 (Johnson & Johnson), or Rituxan (IDEC)), anti-CD5 antibodies (e.g., an anti-CD5 ricin-linked immunoconjugate), anti-CD7 antibodies (e.g., CHH-380 (Novartis)), anti-CD8 antibodies, anti-CD40 ligand monoclonal antibodies (e.g., IDEC-131 (IDEC)), anti-CD52 antibodies (e.g., CAMPATH 1H (Ilex)), anti-CD2 antibodies (e.g., siplizumab (MedImmune, Inc., International Publication Nos. WO 02/098370 and WO 02/069904)), anti-CD11a antibodies (e.g., Xanelim (Genentech)), and anti-B7 antibodies (e.g., IDEC-114) (IDEC))), CTLA4-immunoglobulin, and LFA-3TIP (Biogen, International Publication No. WO 93/08656 and U.S. Pat. No. 6,162,432).

Examples of cytokine receptor modulators include, but are not limited to, soluble cytokine receptors (e.g., the extracellular domain of a TNF-alpha receptor or a fragment thereof, the extracellular domain of an IL-1.beta. receptor or a fragment thereof, and the extracellular domain of an IL-6 receptor or a fragment thereof), cytokines or fragments thereof (e.g., interleukin IL-2, IL-3, IL-4, IL-5, IL-6, IL-7, IL-8, IL-9, IL-10, IL-11, IL-12, IL-13, IL-15, IL-23, TNF-alpha, TNF-beta, interferon (IFN)-alpha, IFN-beta, IFN-gamma, and GM-CSF), anti-cytokine receptor antibodies (e.g., anti-IFN receptor antibodies, anti-IL-2 receptor antibodies (e.g., Zenapax™ (Protein Design Labs)), anti-IL-3 receptor antibodies, anti-IL-4 receptor antibodies, anti-IL-6 receptor antibodies, anti-IL-10 receptor antibodies, anti-IL-12 receptor antibodies, anti-IL-13 receptor antibodies, anti-IL-15 receptor antibodies, and anti-IL-23 receptor antibodies), anti-cytokine antibodies (e.g., anti-IFN antibodies, anti-TNF-.alpha. antibodies, anti-IL-1.beta. antibodies, anti-IL-3 antibodies, anti-IL-6 antibodies, anti-IL-8 antibodies (e.g., ABX-IL-8 (Abgenix)), anti-IL-12 antibodies, anti-IL-13 antibodies, anti-IL-15 antibodies, and anti-IL-23 antibodies).

In some embodiments, a cytokine receptor modulator is IL-3, IL-4, IL-10, or a fragment thereof. In another embodiment, a cytokine receptor modulator is an anti-IL-1-beta antibody, anti-IL-6 antibody, anti-IL-12 receptor antibody, or anti-TNF-alpha antibody. In one embodiment, a TNF-alpha antagonist used in the compositions and methods of the invention is a soluble TNF-alpha receptor. In some embodiments, a TNF-alpha antagonist used in the compositions and methods of the invention is etanercept (ENBREL™; Immunex) or a fragment, derivative or analog thereof. In another embodiment, a TNF-alpha antagonist used in the compositions and methods of the invention is an antibody that immunospecifically binds to TNF-.alpha. In some embodiments, a TNF-alpha antagonist used in the compositions and methods of the invention is infliximab (REMICADE™; Centacor) a derivative, analog or antigen-binding fragment thereof. In another embodiment, a cytokine receptor modulator is the extracellular domain of a TNF-alpha receptor or a fragment thereof. In certain embodiments, a cytokine receptor modulator is not a TNF-alpha antagonist.

In one embodiment, a cytokine receptor modulator is a mast cell modulator. In an alternative embodiment, a cytokine receptor modulator is not a mast cell modulator. Examples of mast cell modulators include, but are not limited to stem cell factor (c-kit receptor ligand) inhibitors (e.g., mAb 7H6, mAb 8H7a, pAb 1337, FK506, CsA, dexamthasone, and fluconcinonide), c-kit receptor inhibitors (e.g., STI 571 (formerly known as CGP 57148B)), mast cell protease inhibitors (e.g., GW-45, GW-58, wortmannin, LY 294002, calphostin C, cytochalasin D, genistein, KT5926, staurosproine, and lactoferrin), relaxin (“RLX”), IgE antagonists (e.g., antibodies rhuMAb-E25 omalizumab, HMK-12 and 6HD5, and mAB Hu-901), IL-3 antagonists, IL-4 antagonists, IL-10 antagonists, and TGF-beta.

An immunomodulatory agent can be selected to interfere with the interactions between the T helper subsets (TH1 or TH2) and B cells to inhibit neutralizing antibody formation. Antibodies that interfere with or block the interactions necessary for the activation of B cells by TH (T helper) cells, and thus block the production of neutralizing antibodies, are useful as immunomodulatory agents in the methods of the invention. For example, B cell activation by T cells requires certain interactions to occur (Durie et al., Immunol. Today, 15(9):406-410 (1994)), such as the binding of CD40 ligand on the T helper cell to the CD40 antigen on the B cell, and the binding of the CD28 and/or CTLA4 ligands on the T cell to the B7 antigen on the B cell. Without both interactions, the B cell cannot be activated to induce production of the neutralizing antibody.

The CD40 ligand (CD40L)-CD40 interaction is a desirable point to block the immune response because of its broad activity in both T helper cell activation and function as well as the absence of redundancy in its signaling pathway. Thus, In some embodiments of the invention, the interaction of CD40L with CD40 is transiently blocked at the time of administration of one or more of the immunomodulatory agents. This can be accomplished by treating with an agent which blocks the CD40 ligand on the TH cell and interferes with the normal binding of CD40 ligand on the T helper cell with the CD40 antigen on the B cell. An antibody to CD40 ligand (anti-CD40L) (available from Bristol-Myers Squibb Co; see, e.g., European patent application 555,880, published Aug. 18, 1993) or a soluble CD40 molecule can be selected and used as an immunomodulatory agent in accordance with the methods of the invention.

An immunomodulatory agent can be selected to inhibit the interaction between TH1 cells and cytotoxic T lymphocytes (“CTLs”) to reduce the occurrence of CTL-mediated killing. An immunomodulatory agent can be selected to alter (e.g., inhibit or suppress) the proliferation, differentiation, activity and/or function of the CD4⁺ and/or CD8⁺ T cells. For example, antibodies specific for T cells can be used as immunomodulatory agents to deplete, or alter the proliferation, differentiation, activity and/or function of CD4⁺ and/or CD8⁺ T cells.

In one embodiment of the invention, an immunomodulatory agent that reduces or depletes T cells, preferably memory T cells, is administered to a subject with a respiratory condition in accordance with the methods of the invention. See, e.g., U.S. Pat. No. 4,658,019.

In another embodiment of the invention, an immunomodulatory agent that inactivates CD8⁺ T cells is administered to a subject with a respiratory condition in accordance with the methods of the invention. In some embodiments, anti-CD8 antibodies are used to reduce or deplete CD8⁺ T cells.

In another embodiment, an immunomodulatory agent which reduces or inhibits one or more biological activities (e.g., the differentiation, proliferation, and/or effector functions) of TH0, TH1, and/or TH2 subsets of CD4⁺ T helper cells is administered to a subject with a respiratory condition in accordance with the methods of the invention. One example of such an immunomodulatory agent is IL-4. IL-4 enhances antigen-specific activity of TH2 cells at the expense of the TH1 cell function (see, e.g., Yokota et al, 1986 Proc. Natl. Acad. Sci., USA, 83:5894-5898; and U.S. Pat. No. 5,017,691). Other examples of immunomodulatory agents that affect the biological activity (e.g., proliferation, differentiation, and/or effector functions) of T-helper cells (in particular, TH1 and/or TH2 cells) include, but are not limited to, IL-2, IL-4, IL-5, IL-6, IL-10, IL-12, IL-13, IL-15, IL-23, and interferon (IFN)-gamma.

In another embodiment, an immunomodulatory agent administered to a subject with a respiratory condition in accordance with the methods of the invention is a cytokine that prevents antigen presentation. In some embodiments, an immunomodulatory agent used in the methods of the invention is IL-10. IL-10 also reduces or inhibits macrophage action which involves bacterial elimination.

An immunomodulatory agent can be selected to reduce or inhibit the activation, degranulation, proliferation, and/or infiltration of mast cells. In certain embodiments, the immunomodulatory agent interferes with the interactions between mast cells and mast cell activating agents, including, but not limited to stem cell factors (c-kit ligands), IgE, IL-4, environmental irritants, and infectious agents. In some embodiments, the immunomodulatory agent reduces or inhibits the response of mast cells to environmental irritants such as, but not limited to pollen, dust mites, tobacco smoke, and/or pet dander. In another specific embodiment, the immunomodulatory agent reduces or inhibits the response of mast cells to infectious agents, such as viruses, bacteria, and fungi. Examples of mast cell modulators that reduce or inhibit the activation, degranulation, proliferation, and/or infiltration of mast cells include, but are not limited to, stem cell factor (c-kit receptor ligand) inhibitors (e.g., mAb 7H6, mAb 8H7a, and pAb 1337 (see Mendiaz et al., 1996, Eur J Biochem 293(3):842-849), FK506 and CsA (Ito et al., 1999 Arch Dermatol Res 291(5):275-283), dexamthasone and fluconcinonide (see Finooto et al. J Clin Invest 1997 99(7):1721-1728)), c-kit receptor inhibitors (e.g., STI 571 (formerly known as CGP 57148B) (see Heinrich et al., 2000 Blood 96(3):925-932)), mast cell protease inhibitors (e.g., GW-45 and GW-58 (see see Temkin et al., 2002 J Immunol 169(5):2662-2669), wortmannin, LY 294002, calphostin C, and cytochalasin D (see Vosseller et al., 1997, Mol Biol Cell 1997:909-922), genistein, KT5926, and staurosproine (see Nagai et al. 1995, Biochem Biophys Res Commun 208(2):576-581), and lactoferrin (see He et al., 2003 Biochem Pharmacol 65(6):1007-1015)), relaxin (“RLX”) (see Bani et al., 2002 Int Immunopharmacol 2(8):1195-1294),), IgE antagonists (e.g., antibodies rhuMAb-E25 omalizumab (see Finn et al., 2003 J Allergy Clin Immuno 111(2):278-284; Corren et al., 2003 J Allergy Clin Immuno 111(1):87-90; Busse and Neaville, 2001 Curr Opin Allergy Clin Immuno 1(1):105-108; and Tang and Powell, 2001, Eur J Pediatr 160(12): 696-704), HMK-12 and 6HD5 (see Miyajima et al., 2202 Int Arch Allergy Immuno 128(1):24-32), and mAB Hu-901 (see van Neerven et al., 2001 Int Arch Allergy Immuno 124(1-3):400), IL-3 antagonist, IL-4 antagonists, IL-10 antagonists, and TGF-beta (see Metcalfe et al., 1995, Exp Dermatol 4(4 Pt 2):227-230).

In some embodiments, proteins, polypeptides or peptides (including antibodies) that are utilized as immunomodulatory agents are derived from the same species as the recipient of the proteins, polypeptides or peptides so as to reduce the likelihood of an immune response to those proteins, polypeptides or peptides. In another preferred embodiment, when the subject is a human, the proteins, polypeptides, or peptides that are utilized as immunomodulatory agents are human or humanized.

In accordance with the invention, one or more immunomodulatory agents are administered to a subject with a respiratory condition prior to, subsequent to, or concomitantly with a PPARδ agonist. Preferably, one or more immunomodulatory agents are administered in combination with a PPARδ agonist to a subject with a respiratory condition to reduce or inhibit one or more aspects of the immune response as deemed necessary by one of skill in the art. Any technique well-known to one skilled in the art can be used to measure one or more aspects of the immune response in a particular subject, and thereby determine when it is necessary to administer an immunomodulatory agent to said subject. In some embodiments, a mean absolute lymphocyte count of approximately 500 cells/mm³, preferably 600 cells/mm³, 650 cells/mm³, 700 cells/mm³, 750 cells/mm³, 800 cells/mm³3, 900 cells/mm³, 1000 cells/mm³, 1100 cells/mm³, or 1200 cells/mm³ is maintained in a subject. In another preferred embodiment, a subject with a respiratory condition is not administered an immunomodulatory agent if their absolute lymphocyte count is 500 cells/mm³ or less, 550 cells/mm³ or less, 600 cells/mm³ or less, 650 cells/mm³ or less, 700 cells/mm³ or less, 750 cells/mm³ or less, or 800 cells/mm³ or less.

In some embodiments, one or more immunomodulatory agents are administered in combination with a PPAR agonist to a subject with a respiratory condition so as to transiently reduce or inhibit one or more aspects of the immune response. Such a transient inhibition or reduction of one or more aspects of the immune system can last for hours, days, weeks, or months. Preferably, the transient inhibition or reduction in one or more aspects of the immune response lasts for a few hours (e.g., 2 hours, 4 hours, 6 hours, 8 hours, 12 hours, 14 hours, 16 hours, 18 hours, 24 hours, 36 hours, or 48 hours), a few days (e.g., 3 days, 4 days, 5 days, 6 days, 7 days, or 14 days), or a few weeks (e.g., 3 weeks, 4 weeks, 5 weeks or 6 weeks). The transient reduction or inhibition of one or more aspects of the immune response enhances the prophylactic and/or therapeutic effect(s) of a PPAR agonist.

Nucleic acid molecules encoding proteins, polypeptides, or peptides with immunomodulatory activity or proteins, polypeptides, or peptides with immunomodulatory activity can be administered to a subject with a respiratory condition in accordance with the methods of the invention. Further, nucleic acid molecules encoding derivatives, analogs, or fragments of proteins, polypeptides, or peptides with immunomodulatory activity, or derivatives, analogs, or fragments of proteins, polypeptides, or peptides with immunomodulatory activity can be administered to a subject with a respiratory infection in accordance with the methods of the invention. Preferably, such derivatives, analogs, and fragments retain the immunomodulatory activity of the full-length, wild-type protein, polypeptide, or peptide.

Preferably, agents that are commercially available and known to function as immunomodulatory agents are used in the methods of the invention. The immunomodulatory activity of an agent can be determined in vitro and/or in vivo by any technique well-known to one skilled in the art, including, e.g., by CTL assays, proliferation assays, and immunoassays (e.g. ELISAs) for the expression of particular proteins such as co-stimulatory molecules and cytokines.

### Anti-Inflammatory Agents

Any anti-inflammatory agent, including agents useful in therapies for inflammatory disorders, well-known to one of skill in the art can be used in the compositions and methods of the invention. Non-limiting examples of anti-inflammatory agents include non-steroidal anti-inflammatory drugs (NSAIDs), steroidal anti-inflammatory drugs, anticholinergics (e.g., atropine sulfate, atropine methylnitrate, and ipratropium bromide (ATROVENT™)), beta2-agonists (e.g., abuterol (VENTOLIN™ and PROVENTIL™), bitolterol (TORNALATE™), levalbuterol (XOPONEX™), metaproterenol (ALUPENT™), pirbuterol (MAXAIR™), terbutlaine (BRETHAIRE™ and BRETHINE™), albuterol (PROVENTIL™, REPETABS™, and VOLMAX™), formoterol (FORADIL AEROLIZER™) and salmeterol (SEREVENT™ and SEREVENT DISKUS™)), and methylxanthines (e.g., theophylline (UNIPHYL™, THEO-DUR™, SLO-BID™, AND TEHO-42™)). Examples of NSAIDs include, but are not limited to, aspirin, ibuprofen, celecoxib (CELEBREX™) diclofenac (VOLTAREN™), etodolac (LODINE™), fenoprofen (NALFON™), indomethacin (INDOCIN™), ketoralac (TORADO™), oxaprozin (DAYPRO™), nabumentone (RELAFEN™), sulindac (CLINORILT™, tolmentin (TOLECTIN™), rofecoxib (VIOXX™) naproxen (ALEVE™, NAPROSYN™), ketoprofen (ACTRON™) and nabumetone (RELAFEN™). Such NSAIDs function by inhibiting a cyclooxygenase enzyme (e.g., COX-1 and/or COX-2). Examples of steroidal anti-inflammatory drugs include, but are not limited to, glucocorticoids, dexamethasone (DECADRON™), corticosteroids (e.g., methylprednisolone (MEDROL™)), cortisone, hydrocortisone, prednisone (PREDNISONE™ and DELTASONE™), prednisolone (PRELONE™ and PEDIAPRED™), triamcinolone, azulfidine, and inhibitors of eicosanoids (e.g., prostaglandins, thromboxanes, and leukotrienes (see Table 2, infra, for non-limiting examples of leukotriene and typical dosages of such agents)). In another embodiment, VITAXIN™ (MedImmune, Inc.), NUMAX™ (MedImmune, Inc.), palivizumab (MedImmune, Inc.), siplizumab (MedImmune, Inc.), an anti-EphA2 antibody (preferably that elicits EphA2 signaling) (see U.S. Patent Publication No. US2004/0028685A1, dated Feb. 12, 2004 and U.S. patent application Ser. No. 10/436,783, filed Can 12, 2003, which are both incorporated by reference herein in their entireties) can be useful in therapies for inflammatory disorders.

In certain embodiments, the anti-inflammatory agent is an agent useful in the prevention, management, treatment, and/or amelioration of asthma or one or more symptoms thereof. Non-limiting examples of such agents include adrenergic stimulants (e.g., catecholamines (e.g., epinephrine, isoproterenol, and isoetharine), resorcinols (e.g., metaproterenol, terbutaline, and fenoterol), and saligenins (e.g., salbutamol)), adrenocorticoids, blucocorticoids, corticosteroids (e.g., beclomethadonse, budesonide, flunisolide, fluticasone, triamcinolone, methylprednisolone, prednisolone, and prednisone), other steroids, beta2-agonists (e.g., albtuerol, bitolterol, fenoterol, isoetharine, metaproterenol, pirbuterol, salbutamol, terbutaline, formoterol, salmeterol, and albutamol terbutaline), anti-cholinergics (e.g., ipratropium bromide and oxitropium bromide), IL-4 antagonists (including antibodies), IL-5 antagonists (including antibodies), IL-13 antagonists (including antibodies), PDE4-inhibitor, NF-Kappa-beta inhibitor, VLA-4 inhibitor, CpG, anti-CD23, selectin antagonists (TBC 1269), mast cell protease inhibitors (e.g., tryptase kinase inhibitors (e.g., GW-45, GW-58, and genisteine), phosphatidylinositide-3′ (PI3)-kinase inhibitors (e.g., calphostin C), and other kinase inhibitors (e.g., staurosporine) (see Temkin et al., 2002 J Immunol 169(5):2662-2669; Vosseller et al., 1997 Mol. Biol. Cell 8(5):909-922; and Nagai et al., 1995 Biochem Biophys Res Commun 208(2):576-581)), a C3 receptor antagonists (including antibodies), immunosuppressant agents (e.g., methotrexate and gold salts), mast cell modulator (e.g., cromolyn sodium (INTAC™) and nedocromil sodium (TILADE™)), and mucolytic agents (e.g., acetylcysteine)). In some embodiments, the anti-inflammatory agent is a leukotriene inhibitor (e.g., montelukast (SINGULAIR™), zafirlukast (ACCOLATE™), pranlukast (ONON™), or zileuton (ZYFLO™)).

In certain embodiments, the anti-inflammatory agent is an agent useful in preventing, treating, managing, or ameliorating allergies or one or more symptoms thereof. Non-limiting examples of such agents include antimmediator drugs (e.g., antihistamine, such as Ethanolamine Diphehydramine, Clemastine, Ethylenediamine Tripelennamine, Alkylamine Brompheniramine, Chlorpheniramine, Triprolidine, Phenothiazine Promethazine, piperazine Hydroxyzine, Piperidines Astemizole, Azatadine, Cetirzine, Cyproheptadine, Fexofenadine, Loratidine), corticosteroids, decongestants, sympathomimetic drugs (e.g., alpha-adrenergic and beta-adrenergic drugs), TNX901 (Leung et al., 2003, N Engl J Med 348(11):986-993), IgE antagonists (e.g., antibodies rhuMAb-E25 omalizumab (see Finn et al., 2003 J Allergy Clin Immuno 111(2):278-284; Corren et al., 2003 J Allergy Clin Immuno 111(1):87-90; Busse and Neaville, 2001 Curr Opin Allergy Clin Immuno 1(1):105-108; and Tang and Powell, 2001, Eur J Pediatr 160(12): 696-704), HMK-12 and 6HD5 (see Miyajima et al, 2202 Int Arch Allergy Immuno 128(1):24-32), and mAB Hu-901 (see van Neerven et al., 2001 Int Arch Allergy Immuno 124(1-3):400), theophylline and its derivatives, glucocorticoids, and immunotherapies (e.g., repeated long-term injection of allergen, short course desensitization, and venom immunotherapy).

Anti-inflammatory therapies and their dosages, routes of administration, and recommended usage are known in the art and have been described in such literature as the Physician's Desk Reference (57th ed., 2003) and The Merk Manual (17th ed., 1999).

### Anti-Viral Agents

Any anti-viral agent well-known to one of skill in the art for the treatment, prevention, management, or amelioration of a respiratory condition or a symptom thereof (e.g., viral respiratory infection) can be used in the compositions and methods of the invention. Non-limiting examples of anti-viral agents include proteins, polypeptides, peptides, fusion proteins antibodies, nucleic acid molecules, organic molecules, inorganic molecules, and small molecules that inhibit and/or reduce the attachment of a virus to its receptor, the internalization of a virus into a cell, the replication of a virus, or release of virus from a cell. In particular, anti-viral agents include, but are not limited to, nucleoside analogs (e.g., zidovudine, acyclovir, gangcyclovir, vidarabine, idoxuridine, trifluridine, and ribavirin), foscarnet, amantadine, rimantadine, saquinavir, indinavir, ritonavir, alpha-interferons and other interferons, and AZT.

In specific embodiments, the anti-viral agent is an antibody agent that is immunospecific for a viral antigen. As used herein, the term “viral antigen” includes, but is not limited to, any viral peptide, polypeptide and protein (e.g., RSV F glycoprotein, RSV G glycoprotein, influenza virus neuraminidase, influenza virus hemagglutinin, and herpes simplex virus glycoprotein (e.g., gB, gC, gD, and gE)) that is capable of eliciting an immune response. Antibodies useful in this invention for prevention, management, treatment, and/or amelioration of a viral infectious disease include, but are not limited to, antibodies against antigens of pathogenic viruses, including as examples and not by limitation: adenovirdiae (e.g., mastadenovirus and aviadenovirus), herpesviridae (e.g., herpes simplex virus 1, herpes simplex virus 2, herpes simplex virus 5, and herpes simplex virus 6), leviviridae (e.g., levivirus, enterobacteria phase MS2, allolevirus), poxyiridae (e.g., chordopoxyirinae, parapoxvirus, avipoxvirus, capripoxvirus, leporiipoxvirus, suipoxvirus, molluscipoxvirus, and entomopoxyirinae), papovaviridae (e.g., polyomavirus and papillomavirus), paramyxoviridae (e.g., paramyxovirus, parainfluenza virus 1, mobillivirus (e.g., measles virus), rubulavirus (e.g., mumps virus), pneumonovirinae (e.g., pneumovirus, human respiratory syncytial virus), and metapneumovirus (e.g., avian pneumovirus and human metapneumovirus)), picornaviridae (e.g., enterovirus, rhinovirus, hepatovirus (e.g., human hepatits A virus), cardiovirus, and apthovirus), reoviridae (e.g., orthoreovirus, orbivirus, rotavirus, cypovirus, fijivirus, phytoreovirus, and oryzavirus), retroviridae (e.g., mammalian type B retroviruses, mammalian type C retroviruses, avian type C retroviruses, type D retrovirus group, BLV-HTLV retroviruses, lentivirus (e.g. human immunodeficiency virus 1 and human immunodeficiency virus 2), spumavirus), flaviviridae (e.g., hepatitis C virus), hepadnaviridae (e.g., hepatitis B virus), togaviridae (e.g., alphavirus (e.g., sindbis virus) and rubivirus (e.g., rubella virus)), rhabdoviridae (e.g., vesiculovirus, lyssavirus, ephemerovirus, cytorhabdovirus, and necleorhabdovirus), arenaviridae (e.g., arenavirus, lymphocytic choriomeningitis virus, Ippy virus, and lassa virus), and coronaviridae (e.g., coronavirus and torovirus).

Specific examples of antibodies available useful for the prevention, management, treatment, and/or amelioration of a viral infectious disease include, but are not limited to, PRO542 (Progenics) which is a CD4 fusion antibody useful for the treatment of HIV infection and palivizumab (MedImmune, Inc.; International Publication No. WO 02/43660) which is a humanized antibody useful for treatment of RSV. See also U.S. Provisional Application No. 60/388,920, filed Jun. 14, 2002 entitled “Stabilized Anti-Respiratory Syncytial Virus (RSV) Antibody Formulations (NUMAX™),” U.S. patent application Ser. No. 10/461,863, filed Jun. 13, 2003 entitled “Stabilized Anti-Respiratory Syncytial Virus (RSV) Antibody Formulations (NUMAX™),” and International Pub. No. U.S. Ser. No. 03/18914, filed Jun. 16, 2003 entitled “Stabilized anti-Respiratory Syncytial Virus (RSV) Antibody Formulations (NUMAX™).

In some embodiments, the anti-viral agent used in the compositions and methods of the invention inhibits or reduces a pulmonary or respiratory virus infection, inhibits or reduces the replication of a virus that causes a pulmonary or respiratory infection, or inhibits or reduces the spread of a virus that causes a pulmonary or respiratory infection to other cells or subjects. In another specific embodiment, the anti-viral agent used in the compositions and methods of the invention inhibits or reduces infection by RSV, hMPV, or PIV, inhibits or reduces the replication of RSV, hMPV, or PIV, or inhibits or reduces the spread of RSV, hMPV, or PIV to other cells or subjects. Examples of such agents include, but are not limited to, nucleoside analogs, such as zidovudine, acyclovir, gangcyclovir, vidarabine, idoxuridine, trifluridine, and ribavirin, as well as foscarnet, amantadine, rimantadine, saquinavir, indinavir, ritonavir, and the alpha-interferons. See U.S. Provisional Patent Application 60/398,475 filed Jul. 25, 2002, entitled “Methods of Treating and Preventing RSV, HMPV, and PIV Using Anti-RSV, Anti-HMPV, and Anti-PIV Antibodies” and U.S. Patent Pub. No. US 2004/0005544 A1, dated Jan. 8, 2004, entitled “Metapneumovirus Strains and Their Use in Vaccine Formulations and as Vectors For Expression of Antigenic Sequences,” which are both incorporated herein by reference in their entirety.

In preferred embodiments, the viral infection is RSV and the anti-viral antigen is an antibody that immunospecifically binds to an antigen of RSV. In certain embodiments, the anti-RSV-antigen antibody immunospecifically binds to an RSV antigen of the Group A of RSV. In other embodiments, the anti-RSV-antigen antibody immunospecifically binds to an RSV antigen of the Group B of RSV. In other embodiments, the anti-RSV antigen antibody immunospecifically binds to an antigen of RSV of one Group and cross reacts with the analogous antigen of the other Group. In particular embodiments, the anti-RSV-antigen antibody immunospecifically binds to a RSV nucleoprotein, RSV phosphoprotein, RSV matrix protein, RSV small hydrophobic protein, RSV RNA-dependent RNA polymerase, RSV F protein, and/or RSV G protein. In additional specific embodiments, the anti-RSV-antigen antibody binds to allelic variants of a RSV nucleoprotein, a RSV nucleocapsid protein, a RSV phosphoprotein, a RSV matrix protein, a RSV attachment glycoprotein, a RSV fusion glycoprotein, a RSV nucleocapsid protein, a RSV matrix protein, a RSV small hydrophobic protein, a RSV RNA-dependent RNA polymerase, a RSV F protein, a RSV L protein, a RSV P protein, and/or a RSV G protein.

It should be recognized that antibodies that immunospecifically bind to a RSV antigen are known in the art. For example, palivizumab (SYNAGIS®) is a humanized monoclonal antibody presently used for the prevention of RSV infection in pediatric patients. In some embodiments, an antibody to be used with the methods of the present invention is palivizumab or an antibody-binding fragment thereof (e.g., a fragment containing one or more complementarity determining regions (CDRs) and preferably, the variable domain of palivizumab). The amino acid sequence of palivizumab is disclosed, e.g., in Johnson et al., 1997, J. Infectious Disease 176:1215-1224, and U.S. Pat. No. 5,824,307 and International Application Publication No.: WO 02/43660, entitled “Methods of Administering/Dosing Anti-RSV Antibodies for Prophylaxis and Treatment”, by Young et al., which are incorporated herein by reference in their entireties.

One or more antibodies or antigen-binding fragments thereof that bind immunospecifically to a RSV antigen comprise a Fc domain with a higher affinity for the FcRn receptor than the Fc domain of palivizumab can also be used in accordance with the invention. Such antibodies are described in U.S. patent application Ser. No. 10/020,354, filed Dec. 12, 2001, which is incorporated herein by reference in its entireties. Further, one or more of the anti-RSV-antigen antibodies A4B4; P12f2 P12f4; P11d4; Ale9; A12a6; A13c4; A17d4; A4B4; 1X-493L1; FR H3-3F4; M3H9; Y10H6; DG; AFFF; AFFF(1); 6H8; L1-7E5; L2-15B10; A13a11; Alh5; A4B4(1); A4B4-F52S; or A4B4L1FR-S28R can be used in accordance with the invention. These antibodies are disclosed in International Application Publication No.: WO 02/43660, entitled “Methods of Administering/Dosing Anti-RSV Antibodies for Prophylaxis and Treatment”, by Young et al., and U.S. Provisional Patent Application 60/398,475 filed Jul. 25, 2002, entitled “Methods of Treating and Preventing RSV, HMPV, and PIV Using Anti-RSV, Anti-HMPV, and Anti-PIV Antibodies” which are incorporated herein by reference in their entireties.

In certain embodiments, the anti-RSV-antigen antibodies are the anti-RSV-antigen antibodies of or are prepared by the methods of U.S. application Ser. No. 09/724,531, filed Nov. 28, 2000; Ser. No. 09/996,288, filed Nov. 28, 2001; and U.S. Pat. Publication No. US2003/0091584 A1, published Can 15, 2003, all entitled “Methods of Administering/Dosing Anti-RSV Antibodies for Prophylaxis and Treatment”, by Young et al., which are incorporated by reference herein in their entireties. Methods and composition for stabilized antibody formulations that can be used in the methods of the present invention are disclosed in U.S. Provisional Application Nos. 60/388,921, filed Jun. 14, 2002, and 60/388,920, filed Jun. 14, 2002, which are incorporated by reference herein in their entireties.

Anti-viral therapies and their dosages, routes of administration and recommended usage are known in the art and have been described in such literature as the Physician's Desk Reference (57th ed., 2003). Additional information on respiratory viral infections is available in Cecil Textbook of Medicine (18th ed., 1988).

### Anti-Bacterial Agents

Anti-bacterial agents and therapies well-known to one of skill in the art for the prevention, treatment, management, or amelioration of a respiratory condition or one or more symptoms thereof (e.g., a bacterial respiratory infection) can be used in the compositions and methods of the invention. Non-limiting examples of anti-bacterial agents include proteins, polypeptides, peptides, fusion proteins, antibodies, nucleic acid molecules, organic molecules, inorganic molecules, and small molecules that inhibit and/or reduce a bacterial infection, inhibit and/or reduce the replication of bacteria, or inhibit and/or reduce the spread of bacteria to other cells or subjects. Specific examples of anti-bacterial agents include, but are not limited to, antibiotics such as penicillin, cephalosporin, imipenem, axtreonam, vancomycin, cycloserine, bacitracin, chloramphenicol, erythromycin, clindamycin, tetracycline, streptomycin, tobramycin, gentamicin, amikacin, kanamycin, neomycin, spectinomycin, trimethoprim, norfloxacin, rifampin, polymyxin, amphotericin B, nystatin, ketocanazole, isoniazid, metronidazole, and pentamidine.

In certain embodiments, the anti-bacterial agent is an agent that inhibits or reduces a pulmonary or respiratory bacterial infection, inhibits or reduces the replication of a bacteria that causes a pulmonary or respiratory infection, or inhibits or reduces the spread of a bacteria that causes a pulmonary or respiratory infection to other cells or subjects. In cases in which the pulmonary or respiratory bacterial infection is a mycoplasma infection (e.g., pharyngitis, tracheobronchitis, and pneumonia), the anti-bacterial agent is preferably a tetracycline, erythromycin, or spectinomycin. In cases in which the pulmonary or respiratory bacterial infection is tuberculosis, the anti-bacterial agent is preferably rifampcin, isonaizid, pyranzinamide, ethambutol, and streptomycin. In cases in which the pulmonary or respiratory bacterial infection is pneumonia caused by an aerobic gram negative bacilli (GNB), the anti-bacterial agent is preferably penicillin, first, second, or third generation cephalosporin (e.g., cefaclor, cefadroxil, cephalexin, or cephazolin), erythomycin, clindamycin, an aminoglycoside (e.g., gentamicin, tobramycin, or amikacine), or a monolactam (e.g., aztreonam). In cases in which the respiratory infection is recurrent aspiration pneumonia, the anti-bacterial agent is preferably penicillin, an aminoglycoside, or a second or third generation cephalosporin.

Anti-bacterial therapies and their dosages, routes of administration and recommended usage are known in the art and have been described in such literature as the Physician's Desk Reference (57th ed., 2003), Cecil Textbook of Medicine (18th ed., 1988), and The Merk Manual of Diagnosis and Therapy (17th ed. 1999).

### Anti-Fungal Agents

Anti-fungal agents and therapies well known to one of skill in the art for prevention, management, treatment, and/or amelioration of a respiratory condition or one or more symptoms thereof (e.g., a fungal respiratory infection) can be used in the compositions and methods of the invention. Non-limiting examples of anti-fungal agents include proteins, polypeptides, peptides, fusion proteins, antibodies, nucleic acid molecules, organic molecules, inorganic molecules, and small molecules that inhibit and/or reduce fungal infection, inhibit and/or reduce the replication of fungi, or inhibit and/or reduce the spread of fungi to other subjects. Specific examples of anti-fungal agents include, but are not limited to, azole drugs (e.g., miconazole, ketoconazole (NIZORAL™), caspofungin acetate (CANCIDAS®), imidazole, triazoles (e.g., fluconazole (DIFLUCAN®)), and itraconazole (SPORANOX®)), polyene (e.g., nystatin, amphotericin B (FUNGIZONEL®), amphotericin B lipid complex (“ABLC”)(ABELCET®), amphotericin B colloidal dispersion (“ABCD”)(AMPHOTEC®), liposomal amphotericin B (AMBISONE®)), potassium iodide (KI), pyrimidine (e.g., flucytosine (ANCOBON®)), and voriconazole (VFEND®).

In certain embodiments, the anti-fungal agent is an agent that inhibits or reduces a respiratory fungal infection, inhibits or reduces the replication of a fungus that causes a pulmonary or respiratory infection, or inhibits or reduces the spread of a fungus that causes a pulmonary or respiratory infection to other subjects. In cases in which the pulmonary or respiratory fungal infection is Blastomyces dermatitidis, the anti-fungal agent is preferably itraconazole, amphotericin B, fluconazole, or ketoconazole. In cases in which the pulmonary or respiratory fungal infection is pulmonary aspergilloma, the anti-fungal agent is preferably amphotericin B, liposomal amphotericin B, itraconazole, or fluconazole. In cases in which the pulmonary or respiratory fungal infection is histoplasmosis, the anti-fungal agent is preferably amphotericin B, itraconazole, fluconazole, or ketoconazole. In cases in which the pulmonary or respiratory fungal infection is coccidioidomycosis, the anti-fungal agent is preferably fluconazole or amphotericin B. In cases in which the pulmonary or respiratory fungal infection is cryptococcosis, the anti-fungal agent is preferably amphotericin B, fluconazole, or combination of the two agents. In cases in which the pulmonary or respiratory fungal infection is chromomycosis, the anti-fungal agent is preferably itraconazole, fluconazole, or flucytosine. In cases in which the pulmonary or respiratory fungal infection is mucormycosis, the anti-fungal agent is preferably amphotericin B or liposomal amphotericin B. In cases in which the pulmonary or respiratory fungal infection is pseudoallescheriasis, the anti-fungal agent is preferably itraconazole or miconazole. Anti-fungal therapies and their dosages, routes of administration, and recommended usage are known in the art and have been described in such literature as Dodds et al., 2000 Pharmacotherapy 20(11) 1335-1355, the Physician's Desk Reference (57th ed., 2003) and the Merk Manual of Diagnosis and Therapy (17th ed., 1999).

### Uses of PPAR Agonists

In some embodiments, a pharmaceutical composition comprising one or more PPAR agonist is administered to a subject, preferably a human, to prevent, treat, manage, or ameliorate a respiratory infection. In accordance with the invention, pharmaceutical compositions of the invention can also comprise one or more prophylactic or therapeutic agents which are currently being used, have been used, or are known to be useful in the prevention, treatment, management, or amelioration of a respiratory condition or one or more symptoms thereof.

### Compositions & Methods of Administering Therapies

The invention also provides compositions for the prevention, treatment, management, or amelioration of an infection, a respiratory condition or one or more symptoms thereof. In some embodiments, the composition comprises one or more PPAR agonists. In some embodiments, the composition comprises one or more PPARδ agonists and one or more prophylactic or therapeutic agents other than PPAR agonists, said prophylactic or therapeutic agents known to be useful for or having been or currently being used in the prevention, treatment, management, or amelioration of an infection, respiratory condition or one or more symptoms thereof. In some embodiments, the composition comprises one or more antibodies that are PPAR agonists. In some other embodiments, the composition comprises one or more antibodies that are PPAR agonists and one or more prophylactic or therapeutic agents other than PPAR agonists, said prophylactic or therapeutic agents known to be useful for or having been or currently being used in the prevention, treatment, management, or amelioration of a respiratory condition or one or more symptoms thereof.

In some embodiments, the composition comprises one or more PPAR agonists and one or immunomodulatory agents. In some other embodiments, the composition comprises one or more PPAR agonists and one or more anti-inflammatory agents. In some embodiments, the composition comprises one or more PPAR agonists and one or more mast cell modulators. Exemplary mast cell modulators include, but are not limited to, stem cell factor (c-kit receptor ligand) inhibitors (e.g., mAb 7H6, mAb 8H7a, pAb 1337, FK506, CsA, dexamthasone, and fluconcinonide), c-kit receptor inhibitors (e.g., STI 571 (formerly known as CGP 57148B)), mast cell protease inhibitors (e.g., GW-45, GW-58, wortmannin, LY 294002, calphostin C, cytochalasin D, genistein, KT5926, and staurosproine, and lactoferrin), relaxin (“RLX”), IgE antagonists (e.g., antibodies rhuMAb-E25 omalizumab, HMK-12 and 6HD5, and mAB Hu-901), IL-3 antagonists, IL-4 antagonists, IL-10 antagonists, and TGF-beta. In some embodiments, the composition comprises one or more PPAR agonists and one or more anti-viral agents. In some other embodiments, the composition comprises one or more PPAR agonists and one or more anti-bacterial agents. In some embodiments, the composition comprises one or more PPAR agonists and one or more anti-fungal agents.

In some embodiments, the composition comprises one or more PPAR agonists and any combination of one, two, three, or more of each of the following prophylactic or therapeutic agents: an immunomodulatory agent, an anti-inflammatory agent, a mast cell modulator, an anti-viral agent, an anti-bacterial agent, and an anti-fungal agent.

In another embodiment, the composition comprises one or more PPARδ agonists and VITAXIN™, siplizumab, palivizumab (SYNAGIS™; MedImmune, Inc.), an EphA2 inhibitor, or any combination thereof. In accordance with this embodiment, the composition can also comprise of one or more other prophylactic or therapeutic agent known or used to treat, manage, prevent, or ameliorate an infection, a respiratory condition or one or more symptoms thereof.

For administration to a subject, the composition can be a pharmaceutical composition, i.e. a composition described herein further comprising a pharmaceutically acceptable carrier. These pharmaceutically acceptable compositions comprise the composition described herein (e.g., a composition comprising a PPARδ agonist and optionally an effective amount of one or more other prophylactic or therapeutic agents) formulated together with one or more pharmaceutically acceptable carriers (additives) and/or diluents. As described in detail below, the pharmaceutical compositions of the present disclosure can be specifically formulated for administration in solid or liquid form, including those adapted for the following: (1) oral administration, for example, drenches (aqueous or non-aqueous solutions or suspensions), lozenges, dragees, capsules, pills, tablets (e.g., those targeted for buccal, sublingual, and/or systemic absorption), boluses, powders, granules, pastes for application to the tongue; (2) parenteral administration, for example, by subcutaneous, intramuscular, intravenous (e.g., bolus or infusion) or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) transmucosally; or (9) nasally. Additionally, compounds can be implanted into a patient or injected using a drug delivery system. See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. No. 3,773,919; and U.S. Pat. No. 35 3,270,960, content of all of which is herein incorporated by reference.

As used herein, the term “pharmaceutically acceptable” or “pharmacologically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. Moreover, for animal (e.g., human) administration, it will be understood that compositions should meet sterility, pyrogenicity, general safety and purity standards as required by FDA Office of Biological Standards. Accordingly, in some embodiments, the term “pharmaceutically acceptable” means approved by a regulatory agency of the Federal or a state government or listed in the U.S. Pharmacopeia or other generally recognized pharmacopeia for use in animals, and more particularly in humans.

As used herein, the term “pharmaceutically acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, including those of petroleum, animal, vegetable or synthetic origin, such as peanut oil, cottonseed oil, safflower oil, sesame oil, olive oil, corn oil, soybean oil, mineral oil, and the like; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alchols, such as ethanol; and (23) other non-toxic compatible substances employed in pharmaceutical formulations. Wetting agents, coloring agents, release agents, coating agents, disintegrating agents, binders, sweetening agents, flavoring agents, perfuming agents, protease inhibitors, plasticizers, emulsifiers, stabilizing agents, viscosity increasing agents, film forming agents, solubilizing agents, surfactants, preservative and antioxidants can also be present in the formulation. The terms such as “excipient”, “carrier”, “pharmaceutically acceptable carrier” or the like are used interchangeably herein. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like

The compositions of the invention include, but are not limited to, bulk drug compositions useful in the manufacture of pharmaceutical compositions (e.g., impure or non-sterile compositions) and pharmaceutical compositions (i.e., compositions that are suitable for administration to a subject or patient) which can be used in the preparation of unit dosage forms. Such compositions comprise a prophylactically or therapeutically effective amount of a prophylactic and/or therapeutic agent disclosed herein or a combination of those agents and a pharmaceutically acceptable carrier. Preferably, compositions of the invention are pharmaceutical compositions and comprise an effective amount of one or more PPARδ agonists, a pharmaceutically acceptable carrier, and, optionally, an effective amount of another prophylactic or therapeutic agent.

Generally, the ingredients of compositions of the invention are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients can be mixed prior to administration.

The compositions of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

Various delivery systems are known and can be used to administer one or more PPARδ agonists or the combination of one or more PPARδ agonists and a prophylactic agent or therapeutic agent useful for preventing, managing, treating, or ameliorating an infection, a respiratory condition or one or more symptoms thereof, e.g., encapsulation in liposomes, microparticles, microcapsules, recombinant cells capable of expressing the antibody or antibody fragment, receptor-mediated endocytosis (see, e.g., Wu and Wu, J. Biol. Chem. 262:4429-4432 (1987)), construction of a nucleic acid as part of a retroviral or other vector, etc. Methods of administering a prophylactic or therapeutic agent of the invention include, but are not limited to, parenteral administration (e.g., intradermal, intramuscular, intraperitoneal, intravenous and subcutaneous), epidurala administration, intratumoral administration, and mucosal administration (e.g., intranasal and oral routes). In addition, pulmonary administration can be employed, e.g., by use of an inhaler or nebulizer, and formulation with an aerosolizing agent. See, e.g., U.S. Pat. Nos. 6,019,968, 5,985,320, 5,985,309, 5,934,272, 5,874,064, 5,855,913, 5,290,540, and 4,880,078; and PCT Publication Nos. WO 92/19244, WO 97/32572, WO 97/44013, WO 98/31346, and WO 99/66903, each of which is incorporated herein by reference their entirety. In one embodiment, an PPARδ agonist, combination therapy, or a composition of the invention is administered using Alkermes AIR™ pulmonary drug delivery technology (Alkermes, Inc., Cambridge, Mass.). In some embodiments, prophylactic or therapeutic agents of the invention are administered intramuscularly, intravenously, intratumorally, orally, intranasally, pulmonary, or subcutaneously. The prophylactic or therapeutic agents can be administered by any convenient route, for example by infusion or bolus injection, by absorption through epithelial or mucocutaneous linings (e.g., oral mucosa, rectal and intestinal mucosa, etc.) and can be administered together with other biologically active agents. Administration can be systemic or local.

In some embodiments, it can be desirable to administer the prophylactic or therapeutic agents of the invention locally to the area in need of treatment; this can be achieved by, for example, and not by way of limitation, local infusion, by injection, or by means of an implant, said implant being of a porous or non-porous material, including membranes and matrices, such as sialastic membranes, polymers, fibrous matrices (e.g., Tissue1™), or collagen matrices. In one embodiment, an effective amount of one or more PPARδ agonists is administered locally to the affected area to a subject to prevent, treat, manage, and/or ameliorate a respiratory condition or a symptom thereof. In another embodiment, an effective amount of one or more PPARδ agonists is administered locally to the affected area in combination with an effective amount of one or more therapies (e.g., one or more prophylactic or therapeutic agents) other than an PPARδ agonist of a subject to prevent, treat, manage, and/or ameliorate a respiratory condition or one or more symptoms thereof. In another embodiment, an effective amount of a therapy such as a mast cell modulator (e.g., astem cell factor (c-kit receptor ligand) inhibitor (e.g., mAb 7H6, mAb 8H7a, pAb 1337, FK506, CsA, dexamthasone, and fluconcinonide), a c-kit receptor inhibitor (e.g., STI 571 (formerly known as CGP 57148B)) and a mast cell protease inhibitor (e.g., GW-45, GW-58, wortmannin, LY 294002, calphostin C, cytochalasin D, genistein, KT5926, staurosproine, and lactoferrin), and relaxin (“RLX”)) is administered locally to the affected area in a subject to prevent, treat, manage, and/or ameliorate a respiratory condition or one or more symptoms thereof.

In yet another embodiment, the prophylactic or therapeutic agent can be delivered in a controlled release or sustained release system. In one embodiment, a pump can be used to achieve controlled or sustained release (see Langer, supra; Sefton, 1987, CRC Crit. Ref. Biomed. Eng. 14:20; Buchwald et al., 1980, Surgery 88:507; Saudek et al., 1989, N. Engl. J. Med. 321:574). In another embodiment, polymeric materials can be used to achieve controlled or sustained release of the therapies of the invention (see e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J., Macromol. Sci. Rev. Macromol. Chem. 23:61; see also Levy et al., 1985, Science 228:190; During et al., 1989, Ann. Neurol. 25:351; Howard et al., 1989, J. Neurosurg. 7 1:105); U.S. Pat. No. 5,679,377; U.S. Pat. No. 5,916,597; U.S. Pat. No. 5,912,015; U.S. Pat. No. 5,989,463; U.S. Pat. No. 5,128,326; PCT Publication No. WO 99/15154; and PCT Publication No. WO 99/20253. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In some embodiments, the polymer used in a sustained release formulation is inert, free of leachable impurities, stable on storage, sterile, and biodegradable. In yet another embodiment, a controlled or sustained release system can be placed in proximity of the prophylactic or therapeutic target, thus requiring only a fraction of the systemic dose (see, e.g., Goodson, in Medical Applications of Controlled Release, supra, vol. 2, pp. 115-138 (1984)).

Controlled release systems are discussed in the review by Langer (1990, Science 249: 1527-1533). Any technique known to one of skill in the art can be used to produce sustained release formulations comprising one or more therapeutic agents of the invention. See, e.g., U.S. Pat. No. 4,526,938, PCT publication WO 91/05548, PCT publication WO 96/20698, Ning et al., 1996, “Intratumoral Radioimmunotheraphy of a Human Colon Cancer Xenograft Using a Sustained-Release Gel,” Radiotherapy & Oncology 39:179-189, Song et al., 1995, “Antibody Mediated Lung Targeting of Long-Circulating Emulsions,” PDA Journal of Pharmaceutical Science & Technology 50:372-397, Cleek et al., 1997, “Biodegradable Polymeric Carriers for a bFGF Antibody for Cardiovascular Application,” Pro. Intl Symp. Control. Rel. Bioact. Mater. 24:853-854, and Lam et al, 1997, “Microencapsulation of Recombinant Humanized Monoclonal Antibody for Local Delivery,” Proc. Int'l. Symp. Control Rel. Bioact. Mater. 24:759-760, each of which is incorporated herein by reference in their entirety.

In some embodiments, where the composition of the invention is a nucleic acid encoding a prophylactic or therapeutic agent, the nucleic acid can be administered in vivo to promote expression of its encoded prophylactic or therapeutic agent, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see, e.g., Joliot et al., 1991, Proc. Natl. Acad. Sci. USA 88:1864-1868). Alternatively, a nucleic acid can be introduced intracellularly and incorporated within host cell DNA for expression by homologous recombination.

A pharmaceutical composition of the invention is formulated to be compatible with its intended route of administration. Examples of routes of administration include, but are not limited to, parenteral, e.g., intravenous, intradermal, subcutaneous, oral, intranasal (e.g., inhalation), transdermal (e.g., topical), transmucosal, and rectal administration. In some embodiments, the composition is formulated in accordance with routine procedures as a pharmaceutical composition adapted for intravenous, subcutaneous, intramuscular, oral, intranasal, or topical administration to human beings. Typically, compositions for intravenous administration are solutions in sterile isotonic aqueous buffer. Where necessary, the composition can also include a solubilizing agent and a local anesthetic such as lignocamne to ease pain at the site of the injection.

If the compositions of the invention are to be administered topically, the compositions can be formulated in the form of an ointment, cream, transdermal patch, lotion, gel, shampoo, spray, aerosol, solution, emulsion, or other form well-known to one of skill in the art. See, e.g., Remington's Pharmaceutical Sciences and Introduction to Pharmaceutical Dosage Forms, 19th ed., Mack Pub. Co., Easton, Pa. (1995). For non-sprayable topical dosage forms, viscous to semi-solid or solid forms comprising a carrier or one or more excipients compatible with topical application and having a dynamic viscosity preferably greater than water are typically employed. Suitable formulations include, without limitation, solutions, suspensions, emulsions, creams, ointments, powders, liniments, salves, and the like, which are, if desired, sterilized or mixed with auxiliary agents (e.g., preservatives, stabilizers, wetting agents, buffers, or salts) for influencing various properties, such as, for example, osmotic pressure. Other suitable topical dosage forms include sprayable aerosol preparations wherein the active ingredient, preferably in combination with a solid or liquid inert carrier, is packaged in a mixture with a pressurized volatile (e.g., a gaseous propellant, such as freon) or in a squeeze bottle. Moisturizers or humectants can also be added to pharmaceutical compositions and dosage forms if desired. Examples of such additional ingredients are well-known in the art.

If the method of the invention comprises intranasal administration of a composition, the composition can be formulated in an aerosol form, spray, mist or in the form of drops. In particular, prophylactic or therapeutic agents for use according to the present invention can be conveniently delivered in the form of an aerosol spray presentation from pressurized packs or a nebuliser, with the use of a suitable propellant (e.g., dichlorodifluoromethane, trichlorofluoromethane, dichlorotetrafluoroethane, carbon dioxide or other suitable gas). In the case of a pressurized aerosol the dosage unit can be determined by providing a valve to deliver a metered amount. Capsules and cartridges (composed of, e.g., gelatin) for use in an inhaler or insufflator can be formulated containing a powder mix of the compound and a suitable powder base such as lactose or starch.

If the method of the invention comprises oral administration, compositions can be formulated orally in the form of tablets, capsules, cachets, gelcaps, solutions, suspensions, and the like. Tablets or capsules can be prepared by conventional means with pharmaceutically acceptable excipients such as binding agents (e.g., pregelatinised maize starch, polyvinylpyrrolidone, or hydroxypropyl methylcellulose); fillers (e.g., lactose, microcrystalline cellulose, or calcium hydrogen phosphate); lubricants (e.g., magnesium stearate, talc, or silica); disintegrants (e.g., potato starch or sodium starch glycolate); or wetting agents (e.g., sodium lauryl sulphate). The tablets can be coated by methods well-known in the art. Liquid preparations for oral administration can take the form of, but not limited to, solutions, syrups or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives, or hydrogenated edible fats); emulsifying agents (e.g., lecithin or acacia); non-aqueous vehicles (e.g., almond oil, oily esters, ethyl alcohol, or fractionated vegetable oils); and preservatives (e.g., methyl or propyl-p-hydroxybenzoates or sorbic acid). The preparations can also contain buffer salts, flavoring, coloring, and sweetening agents as appropriate. Preparations for oral administration can be suitably formulated for slow release, controlled release, or sustained release of a prophylactic or therapeutic agent(s).

The method of the invention can comprise pulmonary administration, e.g., by use of an inhaler or nebulizer, of a composition formulated with an aerosolizing agent. See, e.g., U.S. Pat. Nos. 6,019,968, 5,985,320, 5,985,309, 5,934,272, 5,874,064, 5,855,913, 5,290,540, and 4,880,078; and PCT Publication Nos. WO 92/19244, WO 97/32572, WO 97/44013, WO 98/31346, and WO 99/66903, each of which is incorporated herein by reference their entirety. In some embodiments, an PPARδ agonist, combination therapy, and/or composition of the invention is administered using Alkermes AIR™ pulmonary drug delivery technology (Alkermes, Inc., Cambridge, Mass.).

The method of the invention can comprise administration of a composition formulated for parenteral administration by injection (e.g., by bolus injection or continuous infusion). Formulations for injection can be presented in unit dosage form (e.g., in ampoules or in multi-dose containers) with an added preservative. The compositions can take such forms as suspensions, solutions or emulsions in oily or aqueous vehicles, and can contain formulatory agents such as suspending, stabilizing and/or dispersing agents. In some embodiments, a pharmaceutical composition of the invention is formulated in single dose vials as a sterile liquid that contains 10 mM histidine buffer at pH 6.0 and 150 mM sodium chloride. Each 1.0 mL of solution contains 100 mg of protein, 1.6 mg of histidine and 8.9 mg of sodium chloride in water for optimal stability and solubility.

The methods of the invention can additionally comprise of administration of compositions formulated as depot preparations. Such long acting formulations can be administered by implantation (e.g., subcutaneously or intramuscularly) or by intramuscular injection. Thus, for example, the compositions can be formulated with suitable polymeric or hydrophobic materials (e.g., as an emulsion in an acceptable oil) or ion exchange resins, or as sparingly soluble derivatives (e.g., as a sparingly soluble salt).

The methods of the invention encompass administration of compositions formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

Generally, the ingredients of compositions are supplied either separately or mixed together in unit dosage form, for example, as a dry lyophilized powder or water free concentrate in a hermetically sealed container such as an ampoule or sachette indicating the quantity of active agent. Where the mode of administration is infusion, composition can be dispensed with an infusion bottle containing sterile pharmaceutical grade water or saline. Where the mode of administration is by injection, an ampoule of sterile water for injection or saline can be provided so that the ingredients can be mixed prior to administration.

In particular, the invention also provides that one or more of the prophylactic or therapeutic agents, or pharmaceutical compositions of the invention is packaged in a hermetically sealed container such as an ampoule or sachette indicating the quantity of the agent. In one embodiment, one or more of the prophylactic or therapeutic agents, or pharmaceutical compositions of the invention is supplied as a dry sterilized lyophilized powder or water free concentrate in a hermetically sealed container and can be reconstituted (e.g., with water or saline) to the appropriate concentration for administration to a subject. Preferably, one or more of the prophylactic or therapeutic agents or pharmaceutical compositions of the invention is supplied as a dry sterile lyophilized powder in a hermetically sealed container at a unit dosage of at least 5 mg, more preferably at least 10 mg, at least 15 mg, at least 25 mg, at least 35 mg, at least 45 mg, at least 50 mg, at least 75 mg, or at least 100 mg. The lyophilized prophylactic or therapeutic agents or pharmaceutical compositions of the invention should be stored at between 2 degree C. and 8 degree C. in its original container and the prophylactic or therapeutic agents, or pharmaceutical compositions of the invention should be administered within 1 week, preferably within 5 days, within 72 hours, within 48 hours, within 24 hours, within 12 hours, within 6 hours, within 5 hours, within 3 hours, or within 1 hour after being reconstituted. In an alternative embodiment, one or more of the prophylactic or therapeutic agents or pharmaceutical compositions of the invention is supplied in liquid form in a hermetically sealed container indicating the quantity and concentration of the agent. Preferably, the liquid form of the administered composition is supplied in a hermetically sealed container at least 0.25 mg/ml, more preferably at least 0.5 mg/ml, at least 1 mg/ml, at least 2.5 mg/ml, at least 5 mg/ml, at least 8 mg/ml, at least 10 mg/ml, at least 15 mg/kg, at least 25 mg/ml, at least 50 mg/ml, at least 75 mg/ml or at least 100 mg/ml. The liquid form should be stored at between 2.degree. C. and 8.degree. C. in its original container.

Generally, the ingredients of the compositions of the invention are derived from a subject that is the same species origin or species reactivity as recipient of such compositions. Thus, In some embodiments, human or humanized antibodies are administered to a human patient for therapy or prophylaxis.

### Dosage & Frequency of Administration

The amount of a prophylactic or therapeutic agent or a composition of the present invention which will be effective in the treatment, management, prevention, or amelioration of a respiratory condition or one or more symptoms thereof can be determined by standard clinical. The frequency and dosage will vary according to factors specific for each patient depending on the specific therapy or therapies (e.g., the specific therapeutic or prophylactic agent or agents) administered, the severity of the disorder, disease, or condition, the route of administration, as well as age, body, weight, response, and the past medical history of the patient. For example, the dosage of a prophylactic or therapeutic agent or a composition of the invention which will be effective in the treatment, prevention, management, or amelioration of a respiratory condition or one or more symptoms thereof can be determined by administering the composition to an animal model such as, e.g., the animal models disclosed herein or known to those skilled in the art. In addition, in vitro assays can optionally be employed to help identify optimal dosage ranges. Suitable regimens can be selected by one skilled in the art by considering such factors and by following, for example, dosages reported in the literature and recommended in the Physician's Desk Reference (57th ed., 2003).

Exemplary doses of a small molecule include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram).

In embodiments of the invention wherein antibodies, proteins, polypeptides, peptides and fusion proteins are administered to treat, manage, prevent, or ameliorate a respiratory condition or one or more symptoms thereof, the dosage administered to a patient is typically 0.0001 mg/kg to 100 mg/kg of the patient's body weight. Preferably, the dosage administered to a patient is between 0.0001 mg/kg and 20 mg/kg, 0.0001 mg/kg and 10 mg/kg, 0.0001 mg/kg and 5 mg/kg, 0.0001 and 2 mg/kg, 0.0001 and 1 mg/kg, 0.0001 mg/kg and 0.75 mg/kg, 0.0001 mg/kg and 0.5 mg/kg, 0.0001 mg/kg to 0.25 mg/kg, 0.0001 to 0.15 mg/kg, 0.0001 to 0.10 mg/kg, 0.001 to 0.5 mg/kg, 0.01 to 0.25 mg/kg, or 0.01 to 0.10 mg/kg of the patient's body weight. Generally, human antibodies have a longer half-life within the human body than antibodies from other species due to the immune response to the foreign polypeptides. Thus, lower dosages of human antibodies and less frequent administration is often possible. Further, the dosage and frequency of administration of antibodies or fragments thereof can be reduced by enhancing uptake and tissue penetration of the antibodies by modifications such as, for example, lipidation.

In some embodiments, the dosage administered to a patient will be calculated using the patient's weight in kilograms (kg) multiplied by the dose to be administered in mg/kg. The required volume (in mL) to be given is then determined by taking the mg dose required divided by the concentration of the antibody or fragment thereof in the formulations (100 mg/mL). The final calculated required volume will be obtained by pooling the contents of as many vials as are necessary into syringe(s) to administer the drug. A maximum volume of 2.0 mL of antibody or fragment thereof in the formulations can be injected per site.

In some embodiments, the method of the invention comprises the administration of an PPAR agonist or a composition comprising said agonist to a subject to prevent, treat, manage, or ameliorate a respiratory condition or one or more symptoms thereof in a dosage that is 150 μg/kg or less, preferably 125 μg/kg or less, 100 μg/kg or less, 95 μg/kg or less, 90 μg/kg or less, 85 μg/kg or less, 80 μg/kg or less, 75 μg/kg or less, 70 μg/kg or less, 65 μg/kg or less, 60 μg/kg or less, 55 μg/kg or less, 50 μg/kg or less, 45 μg/kg or less, 40 μg/kg or less, 35 μg/kg or less, 30 μg/kg or less, 25 μg/kg or less, μg/kg or less, 15 μg/kg or less, 10 μg/kg or less, 5 μg/kg or less, 2.5 μg/kg or less, 2 μg/kg or less, 1.5 μg/kg or less, 1 μg/kg or less, 0.5 μg/kg or less, or 0.5 μg/kg or less of a patient's body weight. In another embodiment, the dosage of the PPARδ agonist or composition comprising said agonist that is administered to prevent, treat, manage, or ameliorate a respiratory condition or one or more symptoms thereof in a patient is a unit dose of 0.1 mg to 20 mg, 0.1 mg to 15 mg, 0.1 to 12 mg, 0.1 mg to 10 mg, 0.1 mg to 8 mg, 0.1 mg to 7 mg, 0.1 mg to 5 mg, 0.1 to 2.5 mg, 0.25 mg to 20 mg, 0.25 to 15 mg, 0.25 to 12 mg, 0.25 to 10 mg, 0.25 to 8 mg, 0.25 mg to 7 mg, 0.25 mg to 5 mg, 0.5 mg to 2.5 mg, 1 mg to 20 mg, 1 mg to 15 mg, 1 mg to 12 mg, 1 mg to 10 mg, 1 mg to 8 mg, 1 mg to 7 mg, 1 mg to 5 mg, or 1 mg to 2.5 mg.

In certain embodiments, a subject is administered one or more doses of an effective amount of one or more PPAR agonist, wherein the dose of an effective amount of said PPAR agonist prevents at least 20% to 25%, preferably at least 25% to 30%, at least 30% to 35%, at least 35% to 40%, at least 40% to 45%, at least 45% to 50%, at least 50% to 55%, at least 55% to 60%, at least 60% to 65%, at least 65% to 70%, at least 70% to 75%, at least 75% to 80%, or up to at least 85% of endogenous PPAR from binding to its receptor.

In certain embodiments, a subject is administered one or more doses of an effective amount of one or more PPAR agonist, wherein the dose of an effective amount of said PPAR agonist reduces and/or inhibits mast cell degranulation at least 20% to 25%, preferably at least 25% to 30%, at least 30% to 35%, at least 35% to 40%, at least 40% to 45%, at least 45% to 50%, at least 50% to 55%, at least 55% to 60%, at least 60% to 65%, at least 65% to 70%, at least 70% to 75%, at least 75% to 80%, at least 80 to 85%, at least 85% to 90%, at least 90% to 95%, or at least 95% to 98% relative to a control such as PBS in an in vitro and/or in vivo assay well-known in the art.

In certain embodiments, a subject is administered one or more doses of an effective amount of one or more PPAR agonist, wherein the dose of an effective amount of said PPAR agonist reduces and/or inhibits mast cell activation at least 20% to 25%, preferably at least 25% to 30%, at least 30% to 35%, at least 35% to 40%, at least 40% to 45%, at least 45% to 50%, at least 50% to 55%, at least 55% to 60%, at least 60% to 65%, at least 65% to 70%, at least 70% to 75%, at least 75% to 80%, at least 80 to 85%, at least 85% to 90%, at least 90% to 95%, or at least 95% to 98% relative to a control such as PBS in an in vitro and/or in vivo assay well-known in the art.

In certain embodiments, a subject is administered one or more doses of an effective amount of one or more PPAR agonist, wherein the dose of an effective amount of said PPAR agonist reduces and/or inhibits mast cell proliferation at least 20% to 25%, preferably at least 25% to 30%, at least 30% to 35%, at least 35% to 40%, at least 40% to 45%, at least 45% to 50%, at least 50% to 55%, at least 55% to 60%, at least 60% to 65%, at least 65% to 70%, at least 70% to 75%, at least 75% to 80%, at least 80 to 85%, at least 85% to 90%, at least 90% to 95%, or at least 95% to 98% relative to a control such as PBS in an in vitro and/or in vivo assay well-known in the art.

In certain embodiments, a subject is administered one or more doses of an effective amount of one or more PPAR agonist, wherein the dose of an effective amount of said PPAR agonist reduces and/or inhibits mast cell infiltration in the upper and/or lower respiratory tracts at least 20% to 25%, preferably at least 25% to 30%, at least 30% to 35%, at least 35% to 40%, at least 40% to 45%, at least 45% to 50%, at least 50% to 55%, at least 55% to 60%, at least 60% to 65%, at least 65% to 70%, at least 70% to 75%, at least 75% to 80%, at least 80 to 85%, at least 85% to 90%, at least 90% to 95%, or at least 95% to 98% relative to a control such as PBS in an in vitro and/or in vivo assay well-known in the art.

In other embodiments, a subject is administered one or more doses of an effective amount of one or more PPAR agonists, wherein the dose of an effective amount achieves a serum titer of at least 0.1 μg/ml, at least 0.5 μg/ml, at least 1 μg/ml, at least 2 μg/ml, at least 5 μg/ml, at least 6 μg/ml, at least 10 μg/ml, at least 15 μg/ml, at least 20 μg/ml, at least 25 μg/ml, at least 50 μg/ml, at least 100 μg/ml, at least 125 μg/ml, at least 150 μg/ml, at least 175 μg/ml, at least 200 μg/ml, at least 225 μg/ml, at least 250 μg/ml, at least 275 μg/ml, at least 300 μg/ml, at least 325 μg/ml, at least 350 μg/ml, at least 375 μg/ml, or at least 400 μg/ml of the antagonists. In yet other embodiments, a subject is administered a dose of a prophylactically or therapeutically effective amount of one or more PPARδ agonists to achieve a serum titer of at least 0.1 μg/ml, at least 0.5 μg/ml, at least 1 μg/ml, at least 2 μg/ml, at least μg/ml, at least 6 μg/ml, at least 10 μg/ml, at least 15 μg/ml, at least 20 μg/ml, at least 25 μg/ml, at least 50 μg/ml, at least 100 μg/ml, at least 125 μg/ml, at least 150 μg/ml, at least 175 μg/ml, at least 200 μg/ml, at least 225 μg/ml, at least 250 μg/ml, at least 275 μg/ml, at least 300 μg/ml, at least 325 μg/ml, at least 350 μg/ml, at least 375 μg/ml, or at least 400 μg/ml of the antagonists and a subsequent dose of a prophylactically or therapeutically effective amount of one or more PPAR agonists is administered to maintain a serum titer of at least 0.1 μg/ml, at least 0.5 μg/ml, at least 1 μg/ml, at least, 2 μg/ml, at least 5 μg/ml, at least 6 μg/ml, at least 10 μg/ml, at least 15 μg/ml, at least 20 μg/ml, at least 25 μg/ml, at least 50 μg/ml, at least 100 μg/ml, at least 125 μg/ml, at least 150 μg/ml, at least 175 μg/ml, at least 200 μg/ml, at least 225 μg/ml, at least 250 μg/ml, at least 275 μg/ml, at least 300 μg/ml, at least 325 μg/ml, at least 350 μg/ml, at least 375 μg/ml, or at least 400 μg/ml. In accordance with these embodiments, a subject can be administered 1, 2, 3, 4, 5, 6, 7, 8, 9, 10, 11, 12 or more subsequent doses.

In some embodiments, the invention provides methods of preventing, treating, managing, or treating a respiratory condition or one or more symptoms thereof, said method comprising administering to a subject in need thereof a dose of at least 10 μg, preferably at least 15 μg, at least 20 μg, at least 25 μg, at least 30 μg, at least 35 μg, at least 40 μg, at least 45 μg, at least 50 μg, at least 55 μg, at least 60 μg, at least 65 μg, at least 70 μg, at least 75 μg, at least 80 μg, at least 85 μg, at least 90 μg, at least 95 μg, at least 100 μg, at least 105 μg, at least 110 μg, at least 115 μg, or at least 120 μg of one or more PPAR agonists. In another embodiment, the invention provides a method of preventing, treating, managing, or ameliorating an infection, a respiratory condition or one or more symptoms thereof, said methods comprising administering to a subject in need thereof a dose of at least 10 μg, preferably at least 15 μg, at least 20 μg, at least 25 μg, at least 30 μg, at least 35 μg, at least 40 μg, at least 45 μg, at least 50 μg, at least 55 μg, at least 60 μg, at least 65 μg, at least 70 μg, at least 75 μg, at least 80 μg, at least 85 μg, at least 90 μg, at least 95 μg, at least 100 μg, at least 105 μg, at least 110 μg, at least 115 μg, or at least 120 μg of one or more PPARδ agonists once every 3 days, preferably, once every 4 days, once every 5 days, once every 6 days, once every 7 days, once every 8 days, once every 10 days, once every two weeks, once every three weeks, or once a month.

The present invention provides methods of preventing, treating, managing, or preventing an infection, a respiratory condition or one or more symptoms thereof, said method comprising: (a) administering to a subject in need thereof one or more doses of a prophylactically or therapeutically effective amount of one or more PPAR agonists; and (b) monitoring the plasma level/concentration of the administered PPAR agonist in said subject after administration of a certain number of doses of the said PPAR agonist or antagonists. Moreover, preferably, said certain number of doses is 1, 2, 3, 4, 5, 6, 7, 8, 9, 10, 11, or 12 doses of a prophylactically or therapeutically effective amount one or more PPAR agonists.

In some embodiments, the invention provides a method of preventing, treating, managing, or ameliorating an infection, a respiratory condition or one or more symptoms thereof, said method comprising: (a) administering to a subject in need thereof a dose of at least 10 μg (preferably at least 15 μg, at least 20 μg, at least 25 μg, at least 30 μg, at least 35 μg, at least 40 μg, at least 45 μg, at least 50 μg, at least 55 μg, at least 60 μg, at least 65 μg, at least 70 μg, at least 75 μg, at least 80 μg, at least 85 μg, at least 90 μg, at least 95 μg, or at least 100 μg) of one or more PPAR agonists; and (b) administering one or more subsequent doses to said subject when the plasma level of the PPAR agonist administered in said subject is less than 0.1 μg/ml, preferably less than 0.25 μg/ml, less than 0.5 μg/ml, less than 0.75 μg/ml, or less than 1 μg/ml. In another embodiment, the invention provides a method of preventing, treating, managing, or ameliorating an infection, a respiratory condition or one or more symptoms thereof, said method comprising: (a) administering to a subject in need thereof one or more doses of at least 10 μg (preferably at least 15 μg, at least 20 μg, at least 25 μg, at least 30 μg, at least 35 μg, at least 40 μg, at least 45 μg, at least 50 μg, at least 55 μg, at least 60 μg, at least 65 μg, at least 70 μg, at least 75 μg, at least 80 μg, at least 85 μg, at least 90 μg, at least 95 μg, or at least 100 μg) of one or more PPAR agonists; (b) monitoring the plasma level of the administered PPAR agonist in said subject after the administration of a certain number of doses; and (c) administering a subsequent dose of the PPAR agonist when the plasma level of the administered PPAR agonist or antagonists in said subject is less than 0.1 μg/ml, preferably less than 0.25 μg/ml, less than 0.5 μg/ml, less than 0.75 μg/ml, or less than 1 μg/ml. Preferably, said certain number of doses is 1, 2, 3, 4, 5, 6, 7, 8, 9, 10, 11, or 12 doses of an effective amount of one or more PPAR agonists.

Therapies (e.g., prophylactic or therapeutic agents), other than PPAR agonists, which have been or are currently being used to prevent, treat, manage, or ameliorate a respiratory condition or one or more symptoms thereof can be administered in combination with one or more PPAR agonists according to the methods of the invention to treat, manage, prevent, or ameliorate a respiratory condition or one or more symptoms thereof. Preferably, the dosages of prophylactic or therapeutic agents used in combination therapies of the invention are lower than those which have been or are currently being used to prevent, treat, manage, or ameliorate a respiratory condition or one or more symptoms thereof. The recommended dosages of agents currently used for the prevention, treatment, management, or amelioration of a respiratory condition or one or more symptoms thereof can be obtained from any reference in the art including, but not limited to, Hardman et al., eds., 2001, Goodman & Gilman's The Pharmacological Basis Of Basis Of Therapeutics, 10th ed., McGraw-Hill, New York; Physician's Desk Reference (PDR) 57th ed., 2003, Medical Economics Co., Inc., Montvale, N.J., which are incorporated herein by reference in its entirety.

In various embodiments, the therapies (e.g., prophylactic or therapeutic agents) are administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In preferred embodiments, two or more therapies are administered within the same patient visit.

In certain embodiments, one or more PPAR agonists and one or more other therapies (e.g., prophylactic or therapeutic agents) are cyclically administered. Cycling therapy involves the administration of a first therapy (e.g., a first prophylactic or therapeutic agent) for a period of time, followed by the administration of a second therapy (e.g., a second prophylactic or therapeutic agent) for a period of time, optionally, followed by the administration of a third therapy (e.g., prophylactic or therapeutic agent) for a period of time and so forth, and repeating this sequential administration, i.e., the cycle in order to reduce the development of resistance to one of the therapies, to avoid or reduce the side effects of one of the therapies, and/or to improve the efficacy of the therapies.

In certain embodiments, the administration of the same PPAR agonists can be repeated and the administrations can be separated by at least 1 day, 2 days, 3 days, 5 days, 10 days, 15 days, 30 days, 45 days, 2 months, 75 days, 3 months, or at least 6 months. In other embodiments, the administration of the same therapy (e.g., prophylactic or therapeutic agent) other than an PPAR agonist can be repeated and the administration can be separated by at least at least 1 day, 2 days, 3 days, 5 days, 10 days, 15 days, 30 days, 45 days, 2 months, 75 days, 3 months, or at least 6 months.

Embodiments can be defined by any of the following numbered paragraphs:
1. A method of treating, ameliorating, or preventing an infection, a respiratory condition or one or more symptoms thereof in a subject, said method comprising administering to said subject a therapeutically effective amount of a peroxisome proliferator activated receptor PPAR agonist to a subject in need thereof.
2. The method of paragraph 1, wherein the PPAR agonist is a PPARδ or a PPARγ agonist
3. The method of paragraph 1 or 2, further comprising co-administering a prophylactic or therapeutic agent to the subject,
4. The method of paragraph 3, wherein the prophylactic or therapeutic agent is not a PPAR agonist.
5. The method of paragraph 3 or 4, wherein the prophylactic or therapeutic agent is an immunomodulatory agent, an anti-inflammatory agent, an anti-viral agent, an antibiotic, an antifungal agent or a mast cell modulator.
6. The method of any of paragraphs 1-5, wherein the subject is human.
7. The method of any of paragraphs 1-6, wherein the subject is an immunocompromised or immunosuppressed subject.
8. The method of any of paragraphs 1-7, wherein the subject is a preterm infant, an infant, a child or an elderly person.
9. The method of any of paragraphs 1-8, wherein the subject has bronchopulmonary dysplasia, congenital heart disease, cystic fibrosis or acquired or congenital immunodeficiency.
10. The method of any of paragraphs 1-9, wherein the respiratory infection is a viral infection, a bacterial infection or a fungal infection.
11. The method of any of paragraphs 1-10, wherein the respiratory infection is selected from the group consisting of upper respiratory infection, influenza, croup, respiratory syncytial virus, bronchitis, bronchiolitis and pneumonia.
12. The method of any of paragraphs 1-11, wherein the PPAR agonist is selected from the group consisting of small organic or inorganic molecules; saccharines; oligosaccharides; polysaccharides; peptides; proteins; peptide analogs and derivatives; peptidomimetics; antibodies; antigen binding fragments of antibodies; nucleic acids; nucleic acid analogs and derivatives; an extract made from biological materials; naturally occurring or synthetic compositions; and any combinations thereof.
13. The method of any of paragraphs 1-12, wherein the PPAR agonist is a compound of formula (I):

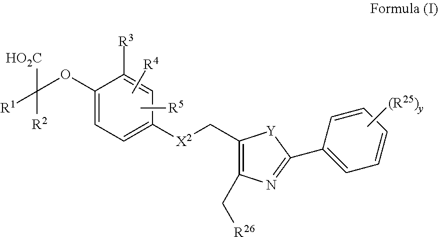

wherein:
R¹ and R² are independently hydrogen or C₁₋₃alkyl;
X² is O, S, or CH₂;
R³, R⁴, and R⁵ are independently H, C₁₋₃alkyl, OCH₃, CF₃, OCF₃, CN, allyl, or halogen;
Y is S or O;
each R²⁵ is independently CH₃, OCH₃, CF₃, or halogen;
each R²⁶ is independently for each occurrence

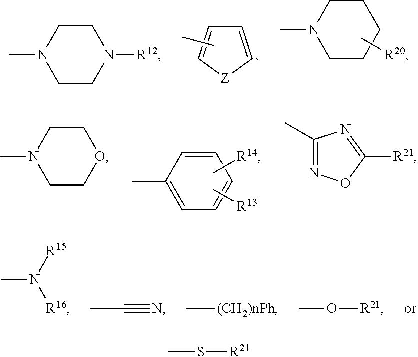

R¹² is independently for each occurrence C₁₋₆alkyl, C₁₋₆alkylenearyl,

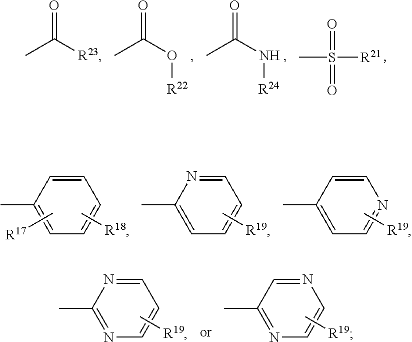

R¹³ and R¹⁴ are independently hydrogen, halogen, CN, perfluoroC₁₋₆alkyl, perfluoro-O—C₁₋₆alkyl, C₁₋₆alkyl, —OC₁₋₆alkyl, —C₁₋₆alkyleneOC₁₋₆alkyl, —SC₁₋₆alkyl, or aryl;
R¹⁵ and R¹⁶ are independently hydrogen, C₁₋₆alkyl, C₃₋₆cycloalkyl optionally substituted with 1 or 2 C₁₋₃alkyl groups, or R¹² as defined above;
R¹⁷ and R¹⁸ are independently hydrogen, halogen, hydroxy, —CN, C₁₋₆alkyl, C₁₋₆ perfluoroalkyl, C₁₋₆acyl, —OC₁₋₆alkyl, perfluoroOC₁₋₆alkyl, or C₁₋₆hydroxyalkyl;
R¹⁹ is independently for each occurrence hydrogen or C₁₋₆alkyl;
R²⁰ is independently for each occurrence C₁₋₆alkyl, aryl, —OC₁₋₆alkyl, hydroxy, C₁₋₆ hydroxyalkyl, or 1-alkoxyC₁₋₆alkyl;
R²¹ is independently for each occurrence C₁₋₆alkyl, —C₁₋₆alkylenearyl, aryl, or aryl-heteroaryl;
R²² is independently for each occurrence independently for each occurrence C₁₋₆alkyl, aryl, or —C₁₋₆alkylenearyl;
R²³ is C₁₋₆alkyl, C₃₋₆cycloalkyl, or aryl;
R²⁴ is independently for each occurrence C₁₋₆alkyl, C₁₋₆alkylenearyl, C₃₋₆cycloalkyl, or aryl;
Z is independently for each occurrence O, N, or S (note that when Z is N, the depicted bond can be attached to the nitrogen in the ring as well as any of the carbons in the ring);
y is 0, 1, 2, 3, 4, or 5; and
n is 1, 2, or 3; and
pharmaceutically acceptable salts, solvates, or hydrolyzable esters thereof.
14. The method of paragraph 12, wherein the compound of formula (I) is:

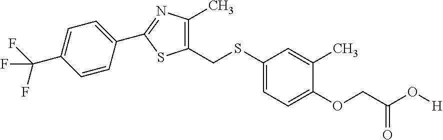 [SMILES: Cc1cc(SCc2sc(-c3ccc(C(F)(F)F)cc3)nc2C)ccc1OCC(=O)O]

### (GW501516).

15. The method of any of paragraphs 1-14, wherein the PPAR agonist is selected from the group consisting of 1-methylethyl 2-(4-(4-chlorobenzoyl)phenoxy)-2-methylpropionate (fenofibrate), ((4-chloro-6-((2,3-dimethylphenyl)amino)-2-pyrimidinyl)thio)acetic acid (WY-14643), (4-(3-(4-acetyl-3-hydroxy-2-propyl)phenoxy)propoxyphenoxy)acetic acid (L-165041), (4-(((2-(3-fluoro-4-(trifluoromethyl)phenyl)-4-methyl-5-thiazolyl)methyl)thio)-2-methylphenoxy)acetic acid (GW-0742), 2-methyl-4-((2R)-2-(3-methyl-5-(4-(trifluoromethyl)phenyl)-2-thienyl)propoxy)-benzenepropionic acid, 2-ethyl-2-(4-(4-(4-(4-methoxyphenyl)-piperazin-1-yl)-2-(4-trifluoromethyl-phenyl)-thiazol-5-ylmethylsulfanyl)-phenoxy)butyric acid (GSK-677954), (4-(3-(3-phenyl-7-propyl-benzofuran-6-yloxy)-propylsulfanyl)-phenyl)acetic acid (L-796449), 2-(4-(3-(1-(2-(2-chloro-6-fluoro-phenyl)-ethyl)-3-(2,3-dichloro-phenyl)-ureido)-propyl)-phenoxy)-2-methylpropionic acid (GW-2433), 2-{2-methyl-4-[({4-methyl-2-[4-(trifluoromethyl)phenyl]-1,3-thiazol-5-yl}methyl)sulfanyl]phenoxy}acetic acid, 2-{2-methyl-4-[({4-methyl-2-[4-(trifluoromethyl)phenyl]-1,3-oxazol-5-yl}methyl)sulfanyl]phenoxy}acetic acid, methyl 2-{4-[({4-methyl-2-[4-(trifluoromethyl)phenyl]-1,3-thiazol-5-yl}methyl)sulfanyl]phenoxy}acetate, 2-{4-[({4-methyl-2-[4-(trifluoromethyl)phenyl]-1,3-thiazol-5-yl}methyl)sulfanyl]phenoxy}acetic acid, (E)-3-[2-methyl-4-({4-methyl-2-[4-(trifluoromethyl)phenyl]-1,3-thiazol-5-yl}methoxy)phenyl]-2-propenoic acid, 2-{3-chloro-4-[({4-methyl-2-[4-(trifluoromethyl)phenyl]-1,3-thiazol-5-yl}methyl)sulfanyl]phenyl}acetic acid, and metabolites of arachidonic acid, e.g., 4-hydroxy-2-nonenal (4-HNE), 4-hydroxydodeca-(2E,6Z)-dienal (4-HDDE), rosiglitazone, pioglitazone, troglitazone, netoglitazone (also known as MCC-555 or isaglitazone or neoglitazone), 5-BTZD, farglitazar, 677954 (GlaxoSmithKline), PLX204 (Plexxikon), LY 519818, L-783483, L-165461, L-16504, and the like.
16. The method of any of paragraphs 1-15, wherein the infection is a viral infection, bacterial infection, or a parasitic infection.
17. The method of any of paragraphs 1-16, wherein the infection is a respiratory, peritoneum, urinary tract, or gut infection.
18. The method of any of paragraphs 1-17, wherein the infection is Streptococcus pneumonia, Staphylococcus aureus, Haemophilus influenzae, Chlcanda pneumoniae, C. psittaci, C. trachomatis, Moraxella (Branhamella) catarrhalis, Legionella pneumophila, or Klebsiella penumoniae infection.
19. A method of treating or ameliorating pneumonia in a human subject suffering therefrom, said method comprising administering to said human subject an effective amount of a PPAR agonist.
20. The method of paragraph 19, wherein the PPAR agonist is a PPARδ or a PPARγ agonist.
21. A composition for treating, ameliorating and/or preventing an infection in a subject comprising an effective amount of at least one effective PPAR agonist.
22. A composition for treating, ameliorating and/or preventing an infection in a subject comprising: an effective amount of at least one effective PPAR agonist; and at least one of an immunomodulatory agent, an anti-inflammatory agent, an anti-viral agent, an antibiotic, an antifungal agent or a mast cell modulator.
23. The composition of paragraph 21 or 22, wherein the PPAR agonist is a PPARδ or a PPARγ agonist.
24. The composition of any of paragraphs 21-23, wherein the infection is a viral infection, bacterial infection, or a parasitic infection.
25. The composition of any of paragraphs 21-24, wherein the infection is a respiratory, peritoneum, urinary tract, or gut infection.
26. A pharmaceutical composition for treating, ameliorating and/or preventing a respiratory infection in a subject comprising an effective amount of at least one effective PPAR agonist and a pharmaceutically acceptable carrier.
27. A pharmaceutical composition for treating, ameliorating and/or preventing a respiratory infection in a subject comprising: an effective amount of at least one effective PPAR agonist; at least one of an immunomodulatory agent, an anti-inflammatory agent, an anti-viral agent, an antibiotic, an antifungal agent or a mast cell modulator; and a pharmaceutically acceptable carrier.
28. The pharmaceutical composition of paragraph 26 or 27, wherein the PPAR agonist is a PPARδ or a PPARγ agonist.
29. The pharmaceutical composition of any of paragraphs 26-28, wherein the infection is a viral infection, bacterial infection, or a parasitic infection
30. The pharmaceutical composition of any of paragraphs 26-29, wherein the infection is a respiratory, peritoneum, urinary tract, or gut infection.
31. Use of a PPAR agonist for the manufacture of a medicament for treating, ameliorating, or preventing an infection, a respiratory condition or one or more symptoms thereof.
32. Use of paragraph 31, wherein the PPAR agonist is a PPARδ or a PPARγ agonist.
33. Use of paragraph 31 or 32, wherein the infection is a viral infection, bacterial infection, or a parasitic infection
34. Use of any of paragraphs 31-33, wherein the infection is a respiratory, peritoneum, urinary tract, or gut infection.

### Some Selected Definitions

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Further, unless otherwise required by context, singular terms shall include pluralities and plural terms shall include the singular.

As used herein the term “comprising” or “comprises” is used in reference to compositions, methods, and respective component(s) thereof, that are useful to the invention, yet open to the inclusion of unspecified elements, whether useful or not. The terms “comprising” and “comprises” include the terms “consisting of” and “consisting essentially of.”

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

Other than in the operating examples, or where otherwise indicated, all numbers expressing quantities of ingredients or reaction conditions used herein should be understood as modified in all instances by the term “about.” The term “about” when used in connection with percentages can mean±1%.

The singular terms “a,” “an,” and “the” include plural referents unless context clearly indicates otherwise. Similarly, the word “or” is intended to include “and” unless the context clearly indicates otherwise.

Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

The terms “decrease”, “reduced”, “reduction”, “decrease” or “inhibit” are all used herein generally to mean a decrease by a statistically significant amount. However, for avoidance of doubt, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g. absent level as compared to a reference sample), or any decrease between 10-100% as compared to a reference level.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “statistically significant” or “significantly” refers to statistical significance and generally means a two standard deviation (2SD) below normal, or lower, concentration of the marker. The term refers to statistical evidence that there is a difference. It is defined as the probability of making a decision to reject the null hypothesis when the null hypothesis is actually true. The decision is often made using the p-value.

As used herein, the terms “effective” and “effectiveness” includes both pharmacological effectiveness and physiological safety. Pharmacological effectiveness refers to the ability of the treatment to result in a desired biological effect in the patient. Physiological safety refers to the level of toxicity, or other adverse physiological effects at the cellular, organ and/or organism level (often referred to as side-effects) resulting from administration of the treatment. “Less effective” means that the treatment results in a therapeutically significant lower level of pharmacological effectiveness and/or a therapeutically greater level of adverse physiological effects.

As used herein, the term “human adult” refers to a human 18 years of age or older.

As used herein, the term “human child” refers to a human between 24 months of age and 18 years of age.

As used herein, the term “immunomodulatory agent” and variations thereof including, but not limited to, immunomodulatory agents, immunomodulants or immunomodulatory drugs, refer to an agent that modulates a host's immune system. In some embodiments, an immunomodulatory agent is an agent that shifts one aspect of a subject's immune response. In certain embodiments, an immunomodulatory agent is an agent that inhibits or reduces a subject's immune system (i.e., an immunosuppressant agent). In certain other embodiments, an immunomodulatory agent is an agent that activates or increases a subject's immune system (i.e., an immunostimulatory agent). In accordance with the invention, an immunomodulatory agent used in the combination therapies of the invention does not include an PPARδ agonist. Immunomodulatory agents include, but are not limited to, small molecules, peptides, polypeptides, proteins, nucleic acids (e.g., DNA and RNA nucleotides including, but not limited to, antisense nucleotide sequences, triple helices, RNAi, and nucleotide sequences encoding biologically active proteins, polypeptides or peptides), antibodies, synthetic or natural inorganic molecules, mimetic agents, and synthetic or natural organic molecules.

As used herein, the term “immunospecifically binds to an antigen” and analogous terms refer to peptides, polypeptides, proteins, fusion proteins and antibodies or fragments thereof that specifically bind to an antigen or a fragment and do not specifically bind to other antigens. A peptide, polypeptide, protein, or antibody that immunospecifically binds to an antigen can bind to other peptides, polypeptides, or proteins with lower affinity as determined by, e.g., immunoassays, BIAcore, or other assays known in the art. Antibodies or fragments that immunospecifically bind to an antigen can be cross-reactive with related antigens. Preferably, antibodies or fragments that immunospecifically bind to an antigen do not cross-react with other antigens. An antibody binds specifically to an antigen when it binds to the antigen with higher affinity than to any cross-reactive antigen as determined using experimental techniques, such as radioimmunoassays (RIAs) and enzyme-linked immunosorbent assays (ELISAs). See, e.g., Paul, ed., 1989, Fundamental Immunology, 2nd ed., Raven Press, New York at pages 332-336 for a discussion regarding antibody specificity.

As used herein, the term “in combination” refers to the use of more than one therapy (e.g., more than one prophylactic agent and/or therapeutic agent). The use of the term “in combination” does not restrict the order in which therapies (e.g., prophylactic and/or therapeutic agents) are administered to a subject with a respiratory condition. A first therapy (e.g., a first prophylactic or therapeutic agent) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., a second prophylactic or therapeutic agent) to a subject with a respiratory condition.

As used herein, the terms “manage,” “managing,” and “management” refer to the beneficial effects that a subject derives from a therapy (e.g., a prophylactic or therapeutic agent), which does not result in a cure of the disease. In certain embodiments, a subject is administered one or more therapies (e.g., one or more prophylactic or therapeutic agents) to “manage” a disease so as to prevent the progression or worsening of the disease.

As used herein, the term “mast cell modulator” refers to an agent which modulates the activation of a mast cell, mast cell degranulation, and/or expression of a particular protein such as a cytokine. Such an agent can directly or indirectly modulate the activation of a mast cell, degranulation of the mast cell, and/or the expression of a particular protein such as a cytokine. Non-limiting examples of mast cell modulators include, but are not limited to, small molecules, peptides, polypeptides, proteins, nucleic acids (e.g., DNA and RNA nucleotides including, but not limited to, antisense nucleotide sequences, triple helices, RNAi, and nucleotide sequences encoding biologically active proteins, polypeptides, or peptides), fusion proteins, antibodies, synthetic or natural inorganic molecules, synthetic or natural organic molecule, or mimetic agents which inhibit and/or reduce the expression, function, and/or activity of a stem cell factor, a mast cell protease, a cytokine (such as IL-3, IL-4, and IL-9), a cytokine receptor (such as IL-3R, IL-4R, and IL-9R), and a stem cell receptor. Other non-limiting examples of mast cell modulators include, but are not limited to small molecules, peptides, polypeptides, proteins, nucleic acids (e.g., DNA and RNA nucleotides including, but not limited to, antisense nucleotide sequences, triple helices, RNAi, and nucleotide sequences encoding biologically active proteins, polypeptides, or peptides), fusion proteins, antibodies, synthetic or natural inorganic molecules, synthetic or natural organic molecule, or mimetic agents which inhibit and/or reduce the expression, function and/or activity of IgE. In certain embodiments, a mast cell modulator is an agent that prevents or reduces the activation of additional mast cells following degranulation of mast cells. In other embodiments, a mast cell modulator is an agent that inhibits or reduces mast cell degranulation. In accordance with the invention, a mast cell modulator used in the combination therapies of the invention does not include a PPAR agonist.

As used herein, the term “isolated” in the context of an organic or inorganic molecule (whether it be a small or large molecule), other than a proteinaceous agent or nucleic acid molecule, refers to an organic or inorganic molecule substantially free of a different organic or inorganic molecule. Preferably, an organic or inorganic molecule is 60%, 65%, 70%, 75%, 80%, 85%, 90%, 95%, or 99% free of a second, different organic or inorganic molecule. In some embodiments, an organic and/or inorganic molecule is isolated.

As used herein, the phrase “pharmaceutically acceptable” means approved by a regulatory agency of the federal or a state government, or listed in the U.S. Pharmacopeia, European Pharmacopeia, or other generally recognized pharmacopeia for use in animals, and more particularly, in humans.

As used herein, the terms “prevent,” “preventing,” and “prevention” refer to the inhibition of the recurrence, onset, development or progression of a respiratory condition or the prevention of the recurrence, onset, or development of one or more symptoms of a respiratory condition in a subject resulting from the administration of a therapy (e.g., a prophylactic or therapeutic agent), or the administration of a combination of therapies (e.g., a combination of prophylactic and/or therapeutic agents).

As used herein, the terms “prophylactic agent” and “prophylactic agents” refer to any agent(s) which can be used in the prevention of a respiratory condition or one or more of the symptoms thereof. In certain embodiments, the term “prophylactic agent” refers to a PPAR agonist. In certain other embodiments, the term “prophylactic agent” refers to an agent other than a PPAR agonist. Preferably, a prophylactic agent is an agent, which is known to be useful to or has been or is currently being used to prevent or impede the onset, development, progression and/or severity of a respiratory condition or one or more symptoms thereof. Prophylactic agents can be characterized as different agents based upon one or more effects that the agents have in vitro and/or in vivo. For example, a mast cell modulator can also be characterized as an immunomodulatory agent.

As used herein, the term “prophylactically effective amount” refers to the amount of a therapy (e.g., prophylactic agent) which is sufficient to result in the prevention of the development, recurrence, onset or progression of a respiratory condition or one or more symptoms thereof, or to enhance or improve the prophylactic effect(s) of another therapy (e.g., a prophylactic agent).

As used herein, a “prophylactic protocol” refers to a regimen for dosing and timing the administration of one or more therapies (e.g., one or more prophylactic agents) that has a prophylactic effect.

A used herein, a “protocol” includes dosing schedules and dosing regimens. The protocols herein are methods of use and include prophylactic and therapeutic protocols.

As used herein, the phrase “side effects” encompasses unwanted and adverse effects of a prophylactic or therapeutic agent. Side effects are always unwanted, but unwanted effects are not necessarily adverse. An adverse effect from a therapy (e.g., a prophylactic or therapeutic agent) might be harmful, uncomfortable, or risky.

As used herein, the term “respiratory condition” refers to a disruption of normal respiratory function and/or activity of tissues, organs, and cells of the respiratory system (e.g., nose, ears, sinuses, throat, trachea, bronchial tubes, and lungs) caused by or associated with an environmental factor or irritant and/or an infectious agent. Respiratory conditions induced by environmental irritants include, but are not limited to, asthma and allergies. Symptoms of a respiratory condition include, but are not limited to, increased mucus production, coughing, bronchoconstriction (i.e., wheezing), fever, sinus pain, lesions in the lung, inflammation of bronchial tubes, sore throat, and/or elevated IgE levels.

As used herein, the term “small molecules” and analogous terms include, but are not limited to, peptides, peptidomimetics, amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such agents.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments of the aspects described herein, the subject is a mammal, e.g., a primate, e.g., a human. The terms, “patient” and “subject” are used interchangeably herein. A subject can be male or female.

Preferably, the subject is a mammal. The mammal can be a human, non-human primate, mouse, rat, dog, cat, horse, or cow, but are not limited to these examples. Mammals other than humans can be advantageously used as subjects that represent animal models of human diseases and disorders. In addition, compounds, compositions and methods described herein can be used to treat domesticated animals and/or pets.

In some embodiments, the methods disclosed herein comprise diagnosing the subject before onset of treatment, e.g., diagnosing for infection or a disease or disorder associated with an infection. Methods of diagnosing a subject for infection are well known in the art. Exemplary methods for diagnosing infections include, but are not limited to, those described, for example, in U.S. Pat. No. 5,571,674; US Patent Application Publication No: US20120257199 and US20130040320; European Patent Publication No: EP0642588, No: EP0643942, No: EP0754243, No: EP0999782, No: EP1430025, No: EP1489416, No: EP1493034, No: EP1609870, No: EP1778880, No: EP1901071, No: EP1915180, No: EP1928614, No: EP2076284; and International Patent Publication No. WO 01/44815, content of all of which is incorporated herein by reference in their entirety. Methods for diagnosing pneumonia are described, for example in, U.S. Pat. No. 8,252,546 and US Patent Application Publication No.: US20120329666 and US20020160407, content of all of which is incorporated herein by reference in their entirety.

For example, pneumonia is diagnosed based on characteristic symptoms and an infiltrate on chest x-ray. Common symptoms of pneumonia include cough, fever, sputum production, tachypnea, and crackles with bronchial breath sounds. Determination of the specific pathogen causing the pneumonia cannot be made in about 30-50% of patients and specimens can be misleading because normal flora can contaminate samples through the upper airways. Special culture techniques, special stains, serologic assays, or lung biopsies can be used for diagnosis.

In some embodiments, the subject is at risk of developing a respiratory condition (e.g., an immunocompromised or immunosuppressed subject). In another embodiment, the subject is not an immunocompromised or immunosuppressed subject. As used herein the terms “immunocompromised,” “immunosuppressed,” and “immunodeficient” refer to a subject whose immune system is deficient or suppressed as indicated by reduced absolute lymphocyte count, depressed CD4(T-helper) lymphocyte count or decreased CD4/CD8(helper/suppressor) ratio which renders the immunocompromised subject unable to evoke an effective immune response. This can render the immunocompromised subject susceptible to a variety of opportunistic infections.

In some embodiments, the subject is a male. In some other embodiments, the subject is a female.

In some embodiments, the subject is an infant or an infant born prematurely. In some embodiments, the subject is a child or an adult.

In some embodiments, the subject is a human infant or a human infant born prematurely. In some other embodiments, the subject is a human child or a human adult. In some embodiments, the subject is a human child with bronchopulmonary dysplasia, congenital heart diseases, or cystic fibrosis. In yet some other embodiments, the subject is an elderly human. In yet still some other embodiments, the subject is a human in an institution or group home, such as, but not limited to, a nursing home.

In some embodiments, the subject has a lymphocyte count that is not under approximately 500 cells/mm³.

As used herein, the term “synergistic” refers to a combination of therapies (e.g., prophylactic or therapeutic agents) which is more effective than the additive effects of any two or more single therapies (e.g., one or more prophylactic or therapeutic agents). A synergistic effect of a combination of therapies (e.g., a combination of prophylactic or therapeutic agents) permits the use of lower dosages of one or more of therapies (e.g., one or more prophylactic or therapeutic agents) and/or less frequent administration of said therapies to a subject with a respiratory condition. The ability to utilize lower dosages of therapies (e.g., prophylactic or therapeutic agents) and/or to administer said therapies less frequently reduces the toxicity associated with the administration of said therapies to a subject without reducing the efficacy of said therapies in the prevention or treatment of a respiratory condition. In addition, a synergistic effect can result in improved efficacy of therapies (e.g., prophylactic or therapeutic agents) in the prevention or treatment of a respiratory condition. Finally, the synergistic effect of a combination of therapies (e.g., prophylactic or therapeutic agents) can avoid or reduce adverse or unwanted side effects associated with the use of any single therapy. In some embodiments, the activity of the combination is at least 5%, at least 10%, at least 15%, at least 20%, at least 25%, at least 30%, at least 40%, at least 50%, at least 60%, at least 70%, at least 80%, at least 90%, at least 1-fold, at least 2-fold, at least 3-fold, at least 4-fold, at least 5-fold, at least 10-fold, at least 50-fold, at least 100-fold or greater than the additive of the individual activities of each component of the combination.

As used herein, the term “T cell receptor modulator” refers to an agent which modulates the phosphorylation of a T cell receptor, the activation of a signal transduction pathway associated with a T cell receptor and/or the expression of a particular protein associated with T cell receptor activity such as a cytokine. Such an agent can directly or indirectly modulate the phosphorylation of a T cell receptor, the activation of a signal transduction pathway associated with a T cell receptor, and/or the expression of a particular protein associated with T cell receptor activity such as a cytokine. Examples of T cell receptor modulators include, but are not limited to, peptides, polypeptides, proteins, fusion proteins and antibodies which immunospecifically bind to a T cell receptor or a fragment thereof. Further, examples of T cell receptor modulators include, but are not limited to, proteins, peptides, polypeptides (e.g., soluble T cell receptors), fusion proteins and antibodies that immunospecifically bind to a ligand for a T cell receptor or fragments thereof.

As used herein, the terms “therapeutic agent” and “therapeutic agents” refer to any agent(s) which can be used in the prevention, treatment, management, or amelioration of a respiratory condition or one or more symptoms thereof. In certain embodiments, the term “therapeutic agent” refers to a PPAR agonist. In certain other embodiments, the term “therapeutic agent” refers an agent other than a PPAR agonist. Preferably, a therapeutic agent is an agent that is known to be useful for, or has been or is currently being used for the prevention, treatment, management, or amelioration of a respiratory condition or one or more symptoms thereof. Therapeutic agents can be characterized as different agents based upon one or more effects the agents have in vivo and/or in vitro, for example, an anti-inflammatory agent can also be characterized as an immunomodulatory agent.

As used herein, the term “therapeutically effective amount” refers to the amount of a therapy (e.g., a PPAR agonist), that is sufficient to reduce the severity of a respiratory condition, reduce the duration of a respiratory condition, ameliorate one or more symptoms of a respiratory condition, prevent the advancement of a respiratory condition, cause regression of a respiratory condition, or enhance or improve the therapeutic effect(s) of another therapy.

The terms “therapies” and “therapy” can refer to any protocol(s), method(s), and/or agent(s) that can be used in the prevention, treatment, management, or amelioration of a respiratory condition or one or more symptoms thereof. In certain embodiments, the terms “therapy” and “therapy” refer to anti-viral therapy, anti-bacterial therapy, anti-fungal therapy, biological therapy, supportive therapy, and/or other therapies useful in treatment, management, prevention, or amelioration of a respiratory condition or one or more symptoms thereof known to skilled medical personnel.

As used herein, the term “therapeutic protocol” refers to a regimen for dosing and timing the administration of one or more therapies (e.g., therapeutic agents) that has a therapeutic effectiveness.

As used herein, the term “administer” refers to the placement of a composition into a subject by a method or route which results in at least partial localization of the composition at a desired site such that desired effect is produced. A compound or composition described herein can be administered by any appropriate route known in the art including, but not limited to, oral or parenteral routes, including intravenous, intramuscular, subcutaneous, transdermal, airway (aerosol), pulmonary, nasal, rectal, and topical (including buccal and sublingual) administration.

Exemplary modes of administration include, but are not limited to, injection, infusion, instillation, inhalation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In preferred embodiments, the compositions are administered by intravenous infusion or injection.

Administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the compounds are formulated into ointments, salves, gels, or creams as generally known in the art.

In jurisdictions that forbid the patenting of methods that are practiced on the human body, the meaning of “administering” of a composition to a human subject shall be restricted to prescribing a controlled substance that a human subject will self-administer by any technique (e.g., orally, inhalation, topical application, injection, insertion, etc.). The broadest reasonable interpretation that is consistent with laws or regulations defining patentable subject matter is intended. In jurisdictions that do not forbid the patenting of methods that are practiced on the human body, the “administering” of compositions includes both methods practiced on the human body and also the foregoing activities.

Generally, the term “treatment” or “treating” is defined as the application or administration of a therapeutic agent to a patient, or application or administration of a therapeutic agent to an isolated tissue or cell line from a patient, said patient having a disease, a symptom of disease or a predisposition toward a disease, with the purpose to cure, heal, alleviate, relieve, alter, remedy, ameliorate, improve or affect the disease, the symptoms of disease or the predisposition toward disease. Thus, treating can include suppressing, inhibiting, preventing, treating, or a combination thereof. Treating refers, inter alia, to increasing time to sustained progression, expediting remission, inducing remission, augmenting remission, speeding recovery, increasing efficacy of or decreasing resistance to alternative therapeutics, or a combination thereof. “Suppressing” or “inhibiting”, refers, inter alia, to delaying the onset of symptoms, preventing relapse to a disease, decreasing the number or frequency of relapse episodes, increasing latency between symptomatic episodes, reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, or a combination thereof. In one embodiment the symptoms are primary, while in another embodiment symptoms are secondary. “Primary” refers to a symptom that is a direct result of a disorder, e.g., diabetes, while, secondary refers to a symptom that is derived from or consequent to a primary cause. Symptoms may be any manifestation of a disease or pathological condition.

Accordingly, as used herein, the term “treatment” or “treating” includes any administration of a compound described herein and includes: (i) preventing the disease from occurring in a subject which may be predisposed to the disease but does not yet experience or display the pathology or symptomatology of the disease; (ii) inhibiting the disease in an subject that is experiencing or displaying the pathology or symptomatology of the diseased (i.e., arresting further development of the pathology and/or symptomatology); or (iii) ameliorating the disease in a subject that is experiencing or displaying the pathology or symptomatology of the diseased (i.e., reversing the pathology and/or symptomatology).

By “treatment”, “prevention” or “amelioration” of a disease or disorder is meant delaying or preventing the onset of such a disease or disorder, reversing, alleviating, ameliorating, inhibiting, slowing down or stopping the progression, aggravation or deterioration the progression or severity of a condition associated with such a disease or disorder. In one embodiment, the symptoms of a disease or disorder are alleviated by at least 5%, at least 10%, at least 20%, at least 30%, at least 40%, or at least 50%.

Specifically, as used herein, the terms “treat,” “treatment,” and “treating” with respect to a respiratory condition refer to the reduction or amelioration of the progression, severity, and/or duration of a respiratory condition or amelioration of one or more symptoms thereof resulting from the administration of one or more therapies (including, but not limited to, the administration of one or more prophylactic or therapeutic agents). In certain embodiments, such terms refer to a reduction in the swelling of organs or tissues, or a reduction in the pain associated with a respiratory condition. In other embodiments, such terms refer to a reduction in the inflammation or constriction of an airway(s) associated with asthma. In other embodiments, such terms refer to a reduction in the replication of an infectious agent, or a reduction in the spread of an infectious agent to other organs or tissues in a subject or to other subjects. In other embodiments, such terms refer to the reduction of the release of inflammatory agents by mast cells, or the reduction of the biological effect of such inflammatory agents.

As used herein, the term “infant” refers to a subject less than 24 months, preferably less than 16 months, less than 6 months, less than 3 months, less than 2 months, or less than 1 month of age. Accordingly, as used herein, the term “human infant” refers to a human less than 24 months, preferably less than 16 months, less than 6 months, less than 3 months, less than 2 months, or less than 1 month of age.

As used herein, the terms “human infant born prematurely,” “preterm infant,” or “premature infant,” or variations thereof refer to a human born at less than 40 weeks of gestational age, preferably less than 35 weeks gestational age, who is less than 6 months old, preferably less than 3 months old, more preferably less than 2 months old, and most preferably less than 1 month old.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow. Further, to the extent not already indicated, it will be understood by those of ordinary skill in the art that any one of the various embodiments herein described and illustrated may be further modified to incorporate features shown in any of the other embodiments disclosed herein.

The disclosure is further illustrated by the following examples which should not be construed as limiting. The examples are illustrative only, and are not intended to limit, in any manner, any of the aspects described herein. The following examples do not in any way limit the invention.

### EXAMPLES

### Example 1

### Regulation of Phagosomal Bacterial Clearance by Myeloid Ppar-δ

Phagosome maturation is a highly coordinated process in which the assemblies of vacuolar ATPases and NADPH oxidases are critical steps for acidification, reactive oxygen species generation and eventually bacterial killing. While the major components constituting the phagosome have been identified, the transcription factors that regulate phagosome function remain unclear. Here we show that the lipid sensing nuclear receptor Ppar-δ controls key genes in ROS production thereby promoting bacterial clearance. Lack of Ppar-δ in the macrophage prevents efficient killing of Streptococcus pneumoniae, the causative bacterium in many cases of respiratory pneumonia. In contrast, Ppar-δ activation improves animal survival after bacterial infection. Our data reveal an unexpected role for Ppar-δ in the control of innate immunity and identify a therapeutic target to improve the outcome of bacterial infections.

It is well established that patients with altered metabolic function such as those with type II diabetes mellitus or elderly people, are more likely to die from bacterial or fungal infections¹⁻³. Respiratory infections are the fifth leading cause of disease in people aged 65 and older⁴. Vaccines have also proven less effective in these populations⁵. To date, several immunological biomarkers have been associated with the increased risk of infections in these susceptible populations. However, the significance of these immunological changes is poorly understood. Some common features found in elderly or people with metabolic diseases include decreased systemic metabolic capacity and increased oxidative stress⁶. These metabolic changes have also been observed in the macrophage. Functionally, myeloid-derived cells from the populations described above have decreased bactericidal capacity and respiratory burst⁸⁻¹². As the immunological process responsible for fending off pathogen attack consumes substantial amounts of energy, identifying signaling nodes that integrate metabolic and immunological functions in immune cells, such as macrophage, may provide novel mechanisms to improve host bactericidal activity in susceptible populations.

Peroxisome proliferator-activated receptors (PPARs) are ligand activated transcription factors critical for maintaining metabolic homeostasis by sensing extracellular nutrients and modulating intracellular metabolism. The Ppar family of receptors consists of Ppar-α (Nr1c1), Ppar-β/δ (Nr1c2) and Ppar-γ (Nr1c3) and is best known for their function in fat metabolism¹³. In the macrophage, Ppar-δ and Ppar-γ regulate oxidative metabolism and anti-inflammatory pathways, such as apoptotic cell clearance¹⁴⁻¹⁶. Given the established role of PPARs in cellular metabolism, immune cell function and organelle regulation, we hypothesized that Ppar-δ may play a role in innate immune response against bacterial infection. Using the gram-positive bacteria Streptococcus pneumoniae (S. pneumoniae), we show here that Ppar-δ regulates phagosomal ROS production and subsequent bacterial killing in the macrophage through transcriptional regulation of genes involved in activation of the NADPH oxidase complex. Our results show pharmacological interventions that increase Ppar-δ activity improve survival in a mouse model of pneumonia.

### Results and Discussion

Myeloid-Specific Ppar-δ Knockout Mice are More Prone to Death Caused by S. pneumoniae Infection:

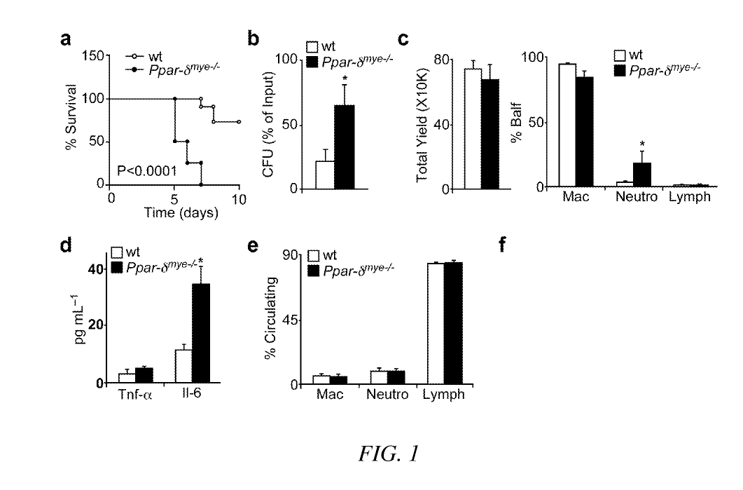

The nuclear receptor Ppar-δ has been shown to regulate macrophage function in immune-metabolism. To assess its role in pathologies associated with the innate immune response, myeloid-specific Ppar-δ knockout mice (Ppar-δ^{mye-/-}; Ppar-δ^{fl/fl}×LysM-cre) and wild-type controls (wt; Ppar-δ^{fl/fl}) were infected intranasally with S. pneumoniae and survival was monitored over 10 days post infection. Remarkable, while most wt mice survived at the relatively low bacteria load, all Ppar-δ^{mye-/-} mice died in 7 days (FIG. 1a). This phenotype was associated with increased bacterial counts (colony-forming units, CFUs) in the bronchoalveolar lavage fluid (Balf) of Ppar-δ^{mye-/-} mice, compared to wt animals (FIG. 1b). Total Balf cell numbers were similar between wt and Ppar-δ^{mye-/-} mice, ruling out a defect in immune cell recruitment (FIG. 1c). However, there was an increase in the neutrophil population and Il-6 concentration in Balf of Ppar-δ^{mye-/-} mice (FIGS. 1c and 1d), indicative of increased inflammation and consistent with the higher bacterial counts in their lungs. No differences were observed in the circulating population of macrophage, neutrophil and lymphocyte (FIG. 1e). These data suggest Ppar-δ^{mye-/-} mice have defects in clearing bacterial infection in a mouse model of pneumonia.

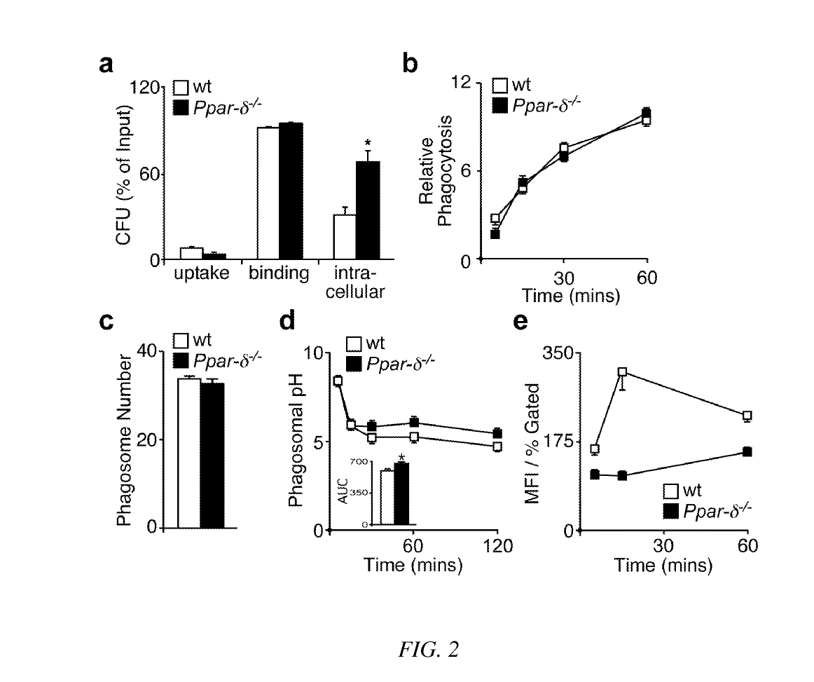

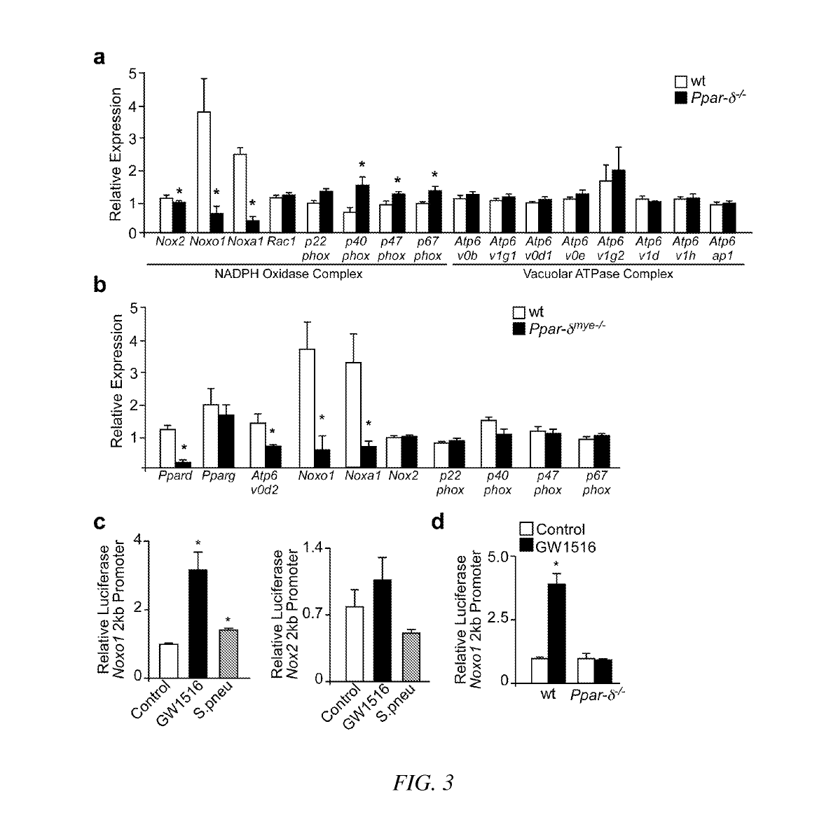

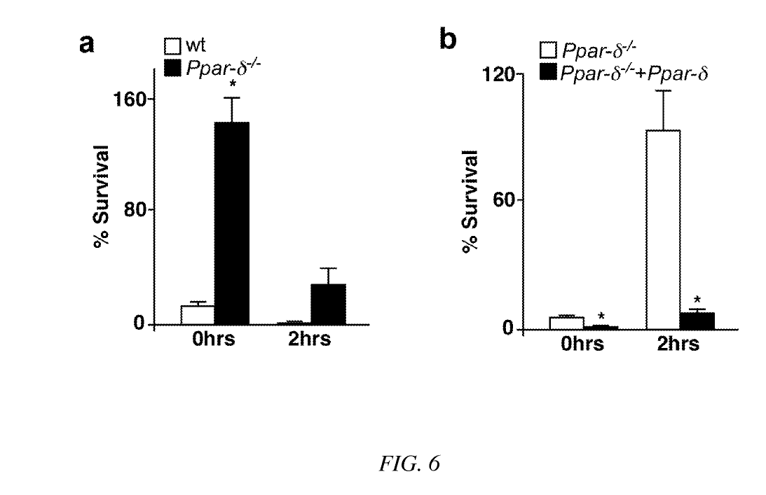

To determine the mechanism underlying the reduced bacteria clearance in Ppar-δ^{mye-/-} mice, in vitro bacterial killing assays were conducted in bone marrow derived macrophages from wt and Ppar-δ^{mye-/-} mice. Macrophages lacking Ppar-δ had approximately two times as many intracellular bacteria remaining two hours after S. pneumoniae infection, compared to wt macrophages (FIG. 2a). There was no difference in bacteria uptake and binding by macrophages between the two genotypes. Rate of phagocytosis and number of phagosomes generated after uptake of bacteria were also similar (FIGS. 2b and 2c). While there was a moderate alteration in phagosomal acidification (higher pH) in Ppar-δ^{mye-/-} macrophages, a major defect associated with Ppar-δ loss-of-function appeared to be the reduction in ROS production (FIGS. 2d and 2e). These results reveal a novel role for Ppar-δ in bacterial killing through regulation of ROS production. In line with this, a similar defect in clearance of gram-negative bacteria was also observed in Ppar-δ^{-/-} macrophages, compared to wt cells (FIG. 6).

Ppar-δ Regulates Expression of Genes Involved in ROS Production:

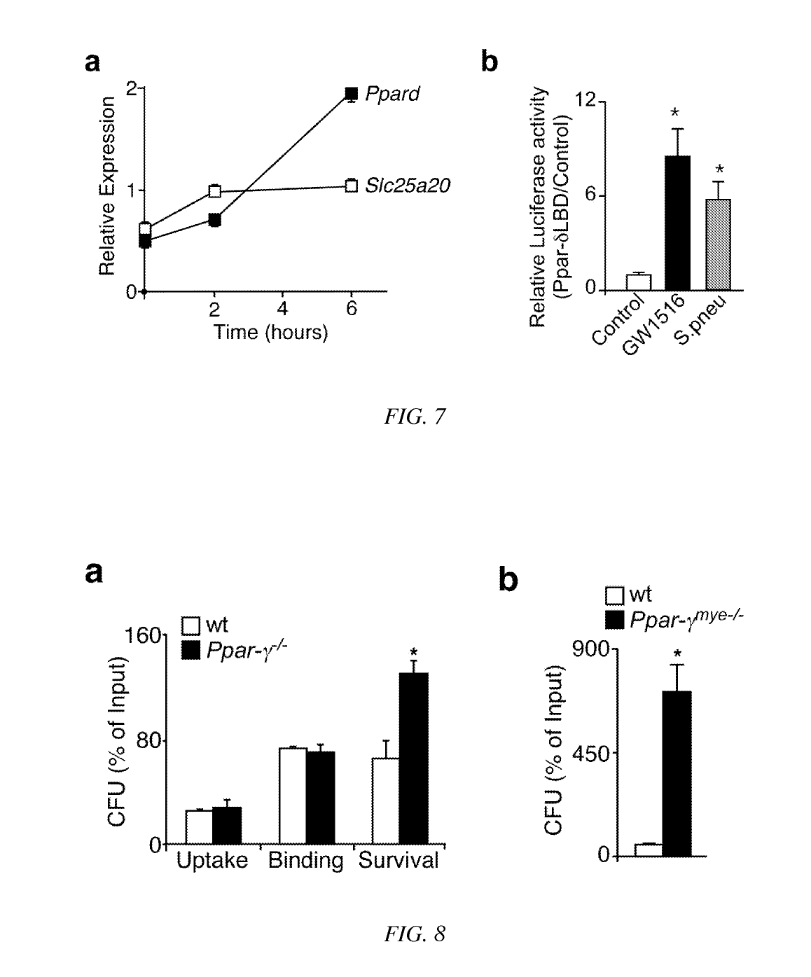

To elucidate downstream targets of Ppar-δ mediating phagosomal ROS production, expression of genes involved in this process was analyzed in stably transformed wt and Ppar-δ^{-/-} macrophages and in alveolar lavage cells from wt and Ppar-δ^{mye-/-} mice after S. pneumoniae infection. Several genes from the NADPH oxidase family, namely Noxo1 and Noxa1, were significantly decreased by Ppar-δ gene deletion (FIGS. 3a and 3b). Interestingly, NADPH oxidase associated proteins, such as p47phox and p22phox, were up-regulated in cultured Ppar-δ^{-/-} macrophages (compared to wt cells) in vitro, but not in vivo. The expression of genes in the vacuolar ATPase complex was mainly unaffected, with the exception of Atp6v0d2, which was down-regulated in alveolar lavage cells from Ppar-δ^{mye-/-} mice (FIG. 3b). To determine whether members of the NADPH complex are transcriptional targets of Ppar-δ, we generated luciferase reporter constructs driven by 2 kb upstream promoters of Noxo1 and Nox2. In RAW267.4 macrophages, treatment with a Ppar-δ agonist, GW501516, or S. pneumoniae increased the promoter activity of Noxo1, but not Nox2 (FIG. 3c). In addition, the ability of GW501516 to induce Noxo1 promoter activity was lost in cells lacking Ppar-δ. These results indicated that the transcriptional activity of Ppar-δ could be modulated during bacteria infection. Indeed, we found the expression of Ppar-δ and its known target Slc25a20 increased after bacteria infection (FIG. 7a). The transactivation activity of Ppar-δ ligand binding domain was also induced in RAW267.4 cells infected with S. pneumoniae (FIG. 7b). Taken together, these findings suggest that Ppar-δ is a transcriptional regulator of phagosomal function in the macrophage.

Noxo1 Gain-of-Function Enhances ROS Production and Bacterial Killing:

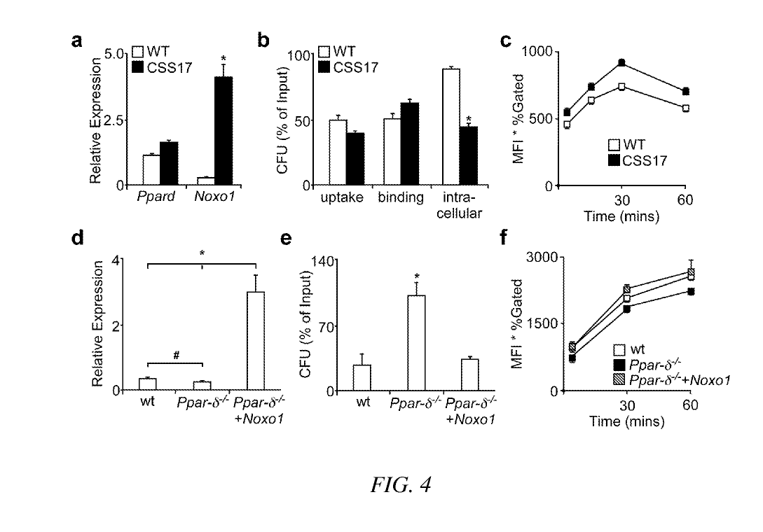

Although Noxo1 has been shown to facilitate generation of ROS by the Nox1 complex, its role in bacterial stimulated ROS production, which is thought to be primarily through the Nox2 complex, has not been described. To determine whether Noxo1 is involved in bacterial killing, we utilized a mouse line that was previously shown to overexpress Noxo1¹⁷. As predicted, Noxo1 expression was increased in bone marrow derived macrophages from CSS17 mice (FIG. 4a). The in vitro bacterial killing assay demonstrated that CSS17 macrophages cleared S. pneumoniae more efficiently than wt cells (FIG. 4b). They also exhibited a higher level of bacterial stimulated ROS production (FIG. 4c). As Noxo1 was down-regulated in Ppar-δ^{-/-} macrophages, we sought to determine whether forced Noxo1 expression could rescue the defect in bacterial killing of Ppar-δ^{-/-} macrophages. To test this possibility, we generated stably transformed wt and Ppar-δ^{-/-} macrophages from bone marrow cells. The mRNA level of Noxo1 was also lower in stable Ppar-δ^{mye-/-} macrophage lines, compared to wt cell lines (FIG. 4d). Retrovirus-mediated Noxo1 gene integration in stable Ppar-δ^{-/-} macrophage lines led to an expected increase in the Noxo1 mRNA level (FIG. 4d). As observed in primary macrophages, stable Ppar-δ^{-/-} macrophage lines had more bacterial counts and reduced bacteria-stimulated ROS burst, compared to wt cells, in the bacterial killing assay (FIGS. 4e and 4f). Forced Noxo1 expression normalized the reduction in bacterial killing and ROS generation in stable Ppar-δ^{-/-} macrophage lines, compared to wt controls. These data suggest that Ppar-δ promotes macrophage bacterial clearance, in part, through transcriptional regulation of Noxo1.

Ppar-δ Activation Improves Survival in a Mouse Model of Pneumonia:

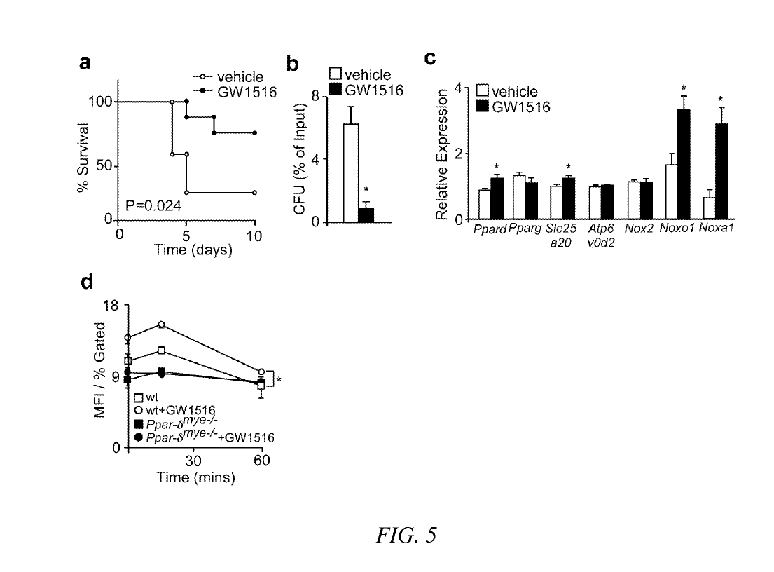

We next sought to explore the therapeutic potential of the Ppar-δ controlled phagosomal function in pathologies associated with infection. For this purpose, wt mice were infected with a higher dose of S. pneumoniae through nasal inhalation, which led to lethality in 75% of mice within 5 days post-injection (FIG. 5a). In contrast, mice that were given a Ppar-δ agonist GW501516 (2 mg kg⁻¹ per day) displayed significant improvement in survival, compared to vehicle-treated mice (FIG. 5a). There was also a delayed onset of death in GW501516 treated animals. Consistent with this finding, the bacteria counts in Balf were substantially lower by GW501516 treatment (FIG. 5b). Gene expression analyses of Balf cells showed an increase in expression of Ppar-δ and its target gene Slc25a20, indicative of Ppar-δ activation in vivo. Levels of Noxo1 and Noxa1 mRNA were also increased (FIG. 5c). To determine whether this regulatory mechanism led to functional consequences, Fc-stimulated phagosomal ROS production was examined ex vivo in isolated alveolar macrophages from wt and Ppar-δ^{mye-/-} mice that have been given GW501516 for 3 days. Similar to our in vitro data, Ppar-δ^{-/-} alveolar macrophages showed reduced levels of Fc-stimulated ROS (FIG. 5d). In addition, GW501516 increased ROS production in a Ppar-δ dependent manner. Taken together, our data implicate the potential use of synthetic Ppar-δ agonists to improve the outcomes of bacteria infection, such as pneumonia.

Without wishing to be bound by a theory, it is noted that the bactericidal activity is not limited to myeloid/macrophage Ppar-δ. Myeloid Ppar-γ, which is a target of the TZD class of insulin sensitizing drugs (such as rosiglitazone and pioglitazone), also has a similar effect in promoting bacterial clearance (FIG. 8).

We investigated the role of lipid sensing nuclear receptors in bacterial killing. We show that Ppar-δ plays a key role in phagosomal ROS production and transcriptional activation of Noxo1. Our data also suggests that Noxo1 is important for efficient bacterial killing. Lack of Ppar-δ in the macrophage contributes to inefficient bacterial killing. In contrast, pharmacological activation of Ppar-δ enhances bacterial clearance and improves the outcome of pneumonia.

Ppar-δ is a lipid sensing nuclear receptor. Despite its role in regulating metabolic processes in other cells, Ppar-δ does not appear to regulate these pathways during bacterial killing. As such, we have elucidated a mechanism by which Ppar-δ regulates phagosomal ROS production. The defects in ROS production lead to alterations in the ability of the macrophage to kill bacteria. This is observed in vitro and in vivo indicating that there is a cell autonomous defect that contributes to the inability of macrophages lacking Ppar-δ to kill bacteria. We show here that the defect arises from an inability of Ppar-δ to regulate Noxo1 transcription. Noxo1 is a homologue of p47phox, an organizer protein that has been shown to associate with the Nox2 complex to mediate proper phagosomal ROS production. Phosphorylation of the AIR domain in response to pathogen ligation of surface receptors activates p47phox allowing for a tight regulation of ROS production in the phagosome¹⁸. Lack of the AIR domain in Noxo1 is thought to lead to constitutive activation and therefore contributes to the activity of the more ubiquitous oxidases, Nox1 and Nox3^{18,19}. To date, a role for Noxo1 in bacterial stimulated ROS production and bacterial killing has not been shown.

Recently it was observed that Noxo1 is expressed in macrophages and that mRNA is induced in the presence of inflammatory agents such as LPS and Tnf-α, suggesting that transcriptional upregulation of Noxo1 may play a role in the bactericidal activity of the macrophage^{20,21}. Several studies showed that Noxo1 can associate with Nox2 and p22phox²²⁻²⁴, although a functional requirement for Noxo1 during bacterial killing was never established in vivo. Our data suggest that Noxo1 is required for efficient bacterial killing and ROS production. Through in vitro assays, we show that Ppar-δ ligand, GW501516, is able to increase activity of the promoter of Noxo1. This effect was lost in cells that did not contain Ppar-δ. Reconstituting Noxo1 in Pparδ^{-/-} macrophages restored the bacterial killing defects we observed. Together our data suggest a mechanism by which PPAR-6 regulated Noxo1 transcriptional activation during bacterial killing regulated bacterial stimulated ROS production and killing.

Neonates, elderly patients, and people with type 2 diabetes have increased rates of morbidity from bacterial and fungal infections compared to a young, healthy population, suggesting a common defect in immune function^{1,25-27}. A previous study showed that mice with higher activity of nuclear receptor co-repressors display decreased Ppar-δ activity and exhibit pathologies associated with aging. Polymorphisms in these same co-repressors are associated with type 2 diabetes and increased insulin resistance²⁸. Age-associated changes in Ppar nuclear protein, mRNA levels, and promoter binding were also observed in rat kidneys^{6,29}. Together, we suggest a novel mechanism to increase host bacterial killing potential to improve killing efficiency. Further studies will determine if ligand treatment post infection can recapitulate these same findings. Additionally, studies should be conducted addressing the ability of combination treatments involving Ppar-δ ligands and antibiotics in the treatment of bacterial infections. While Ppar-δ ligand appears to improve bacterial killing, it is also likely that use of multiple ligands to improve several pathways of host killing could be much more potent. If these pathways regulate bacterial killing in the human macrophage, it would also be exciting to see if similar ligands could be used to treat other types of bacterial, viral, or fungal stresses. In recent years, a lack of novel antibiotics and the rise of multi-drug resistant pathogens have led to the immediate need for novel therapeutics (CDC, WHO). The link between Ppar-δ and phagosomal function provides a novel mechanism to improve host immune function and bacterial killing.

Together, the data presented herein provides a novel mechanism to increase host bacterial killing potential to improve killing efficiency.

### Material and Methods

Bacterial Growth and Manipulation.

Streptococcus pneumoniae, serotype 3 was obtained from American Type Culture Collection. Bacteria were cultured as described previously³⁰. For large volumes of S. pneumoniae, bacteria was cultured in Tryptic Soy Broth for 8 hours at 37° C. without shaking. Fluorescent magnetic beads (BM570, Bangs Laboratory) were conjugated to S. pneumoniae as previously described³¹. S. pneumoniae was heat-killed by incubation at 50° C. for 40 mins. Conjugation of S. pneumoniae to Alexa Fluor 647 and FITC was performed as previously described³²

Animal Experiments.

Ppar-δ floxed animals were generated as described previously^{33,34}. Ppar-δ mice were crossed to lysozyme cre mice (Jackson laboratory) to generate Ppar-δ^{mye-/-} mice. Ppar-δ^{fl/fl} mice were used as wt controls. All mice were maintained on a C57BL/6 background. Mice used for survival studies in FIG. 5 were C57BL/6 mice purchased from Jackson Laboratory. Bone marrow from CSS-17 mice was provided by Dr. Colleen Croniger (Case Western Reserve University). For in vivo killing assays, mice were anesthetized with a ketamine/xylazine solution and infected intranasally with 10⁶ CFU S. pneumoniae. 24 hours post infection, mice were sacrificed with isofluorane and Balf was extracted³⁰. For survival experiments with Ppar-δ^{mye-/-} mice, animals were infected intranasally with 10⁵ CFU S. pneumoniae. C57BL/6 mice used for survival experiments with ligand treatment were infected with 5×10⁵ CFU. Mice were observed for signs of distress twice a day for 2 weeks³⁰. Ligand was gavaged for the indicated amount of time at a dose of 2 mg kg⁻¹ per day. All animal studies were approved by the Harvard Medical Area Standing Committee on Animals.

Primary Cells and Stable Cell Lines.

Bone marrow-derived macrophages were differentiated in L929 conditioned media as previously described¹⁴. Stables lines were created from bone marrow of Ppar-δ^{mye-/-} and control mouse lines using a CreJ2 viral system^{35,36}. A retroviral Cre virus was then used to generate clonal wt and knockout cell lines. Retroviral vectors were also used to generate cell lines overexpressing Ppar-δ. Alveolar macrophages were obtained from mice by lavage after sacrifice.

Functional Assays.

Bacterial killing assays were used for in vitro bacterial killing^{37,38}. Briefly, macrophages were incubated with S. pneumoniae. After 1 hour, cells were washed with PBS to remove uningested bacteria. Cells were allowed to proceed with killing for an additional hour. Colony forming units (CFU) were determined as a measure of bacterial survival. ROS assays were done using FcOxyburst (Invitrogen) according to manufacturers protocols and analyzed by fluorescence activated cell sorting (FACS). Phagocytosis and phagosome number were determined by FACS using Alexa Fluor 647 labeled heat killed S. pneumoniae³². pH was determined as previously described³². Briefly, S. pneumoniae was cultured, heat-killed and conjugated with Alexa Fluor 647 (pH insensitive) and fluorescein isothiocyanate (FITC; pH sensitive). Macrophages were incubated with labeled bacteria and analyzed by FACS for FITC relative to Alexa Fluor 647 signal. ATP and lactate were measured using commercially available kits. Mitotracker (Invitrogen) was used to determine mitochondrial number. Glucose uptake was modified from a previously described protocol^{39,40}. Data shown is insulin stimulated normalized to untreated glucose uptake.

Expression Analyses.

For gene expression analyses, we determined relative expression levels using SYBR Green-based real-time qPCR reactions. We used 36B4 expression levels for normalization. For in vitro analyses of infected macrophages, S. pneumoniae conjugated to magnetic beads were used during infection (see Bacterial Growth and Manipulation for details). Cells were infected for 1 hr with conjugated beads. At the end of the experiment, a magnet was used to separate cells that contained intracellular magnetic bacteria. These cells were used for expression analyses.

Statistical Analyses.

Statistical analyses comparing two parameters (between treatments or genotypes) in cell-based work were conducted using the two-tailed Student's t test. Two parameter analyses for samples from in vivo studies (non-gaussian distribution) were determined using the Mann-Whitney test. Statistics for multi-parameter analyses was determined by One-Way ANOVA followed by Bonferroni posthoc tests. Kaplan-Meier statistics were used for the survival curves. Values are presented as mean±s.e.m. For in vitro assays, the mean and s.e.m. were determined from 3-4 biological replicates for one representative experiment. Experiments were repeated at least three times. P<0.05 was considered significant.

### REFERENCES

1. Geerlings, S. E. & Hoepelman, A. I. Immune dysfunction in patients with diabetes mellitus (DM). in FEMS Immunol Med Microbiol, Vol. 26 259-265 (1999).
2. Redmond, H. P., et al. Impaired macrophage function in severe protein-energy malnutrition. Arch Surg 126, 192-196 (1991).
3. Sammalkorpi, K. Glucose intolerance in acute infections. J Intern Med 225, 15-19 (1989).
4. High, K. P. Infection as a cause of age-related morbidity and mortality. Ageing Res Rev 3, 1-14 (2004).
5. Aspinall, R., Del Giudice, G., Effros, R. B., Grubeck-Loebenstein, B. & Sambhara, S. Challenges for vaccination in the elderly. Immun Ageing 4, 9 (2007).
6. Erol, A. The Functions of PPARs in Aging and Longevity. PPAR Res 2007, 39654 (2007).
7. Rosa, L. F., De Almeida, A. F., Safi, D. A. & Curi, R. Metabolic and functional changes in lymphocytes and macrophages as induced by ageing. Physiol Behav 53, 651-656 (1993).
8. Lloberas, J. & Celada, A. Effect of aging on macrophage function. Exp Gerontol 37, 1325-1331 (2002).
9. Sebastian, C., Espia, M., Serra, M., Celada, A. & Lloberas, J. MacrophAging: a cellular and molecular review. Immunobiology 210, 121-126 (2005).
10. Alvarez, E. & Santa Maria, C. Influence of the age and sex on respiratory burst of human monocytes. Mech Ageing Dev 90, 157-161 (1996).
11. Butcher, S. K., et al. Senescence in innate immune responses: reduced neutrophil phagocytic capacity and CD16 expression in elderly humans. J Leukoc Biol 70, 881-886 (2001).
12. Lord, J. M., Butcher, S., Killampali, V., Lascelles, D. & Salmon, M. Neutrophil ageing and immunesenescence. Mech Ageing Dev 122, 1521-1535 (2001).
13. Lee, C. H., Olson, P. & Evans, R. M. Minireview: lipid metabolism, metabolic diseases, and peroxisome proliferator-activated receptors. Endocrinology 144, 2201-2207 (2003).
14. Kang, K., et al. Adipocyte-derived Th2 cytokines and myeloid PPARdelta regulate macrophage polarization and insulin sensitivity. Cell Metab 7, 485-495 (2008).
15. Odegaard, J. I., et al. Macrophage-specific PPARgamma controls alternative activation and improves insulin resistance. Nature 447, 1116-1120 (2007).
16. Mukundan, L., et al. PPAR-delta senses and orchestrates clearance of apoptotic cells to promote tolerance. Nature medicine 15, 1266-1272 (2009).
17. Millward, C. A., et al. Genetic factors for resistance to diet-induced obesity and associated metabolic traits on mouse chromosome 17. Mamm Genome 20, 71-82 (2009).
18. Sumimoto, H., Ueno, N., Yamasaki, T., Taura, M. & Takeya, R. Molecular mechanism underlying activation of superoxide-producing NADPH oxidases: roles for their regulatory proteins. Jpn J Infect Dis 57, S24-25 (2004).
19. Dutta, S. & Rittinger, K. Regulation of NOXO1 activity through reversible interactions with p22 and NOXA1. PLoS One 5, e10478.
20. Leto, T. L., Morand, S., Hurt, D. & Ueyama, T. Targeting and regulation of reactive oxygen species generation by Nox family NADPH oxidases. Antioxid Redox Signal 11, 2607-2619 (2009).
21. Lambeth, J. D., Kawahara, T. & Diebold, B. Regulation of Nox and Duox enzymatic activity and expression. Free Radic Biol Med 43, 319-331 (2007).
22. Kawano, M., Miyamoto, K., Kaito, Y., Sumimoto, H. & Tamura, M. Noxa1 as a moderate activator of Nox2-based NADPH oxidase. Arch Biochem Biophys 519, 1-7.
23. Piccoli, C., et al. Bone-marrow derived hematopoietic stem/progenitor cells express multiple isoforms of NADPH oxidase and produce constitutively reactive oxygen species. Biochem Biophys Res Commun 353, 965-972 (2007).
24. Takeya, R., et al. Novel human homologues of p47phox and p67phox participate in activation of superoxide-producing NADPH oxidases. J Biol Chem 278, 25234-25246 (2003).
25. Busse, P. J. & Mathur, S. K. Age-related changes in immune function: effect on airway inflammation. J Allergy Clin Immunol 126, 690-699; quiz 700-691.
26. Hoffman, J. A., et al. Streptococcus pneumoniae infections in the neonate. Pediatrics 112, 1095-1102 (2003).
27. Shivshankar, P., Boyd, A. R., Le Saux, C. J., Yeh, I. T. & Orihuela, C. J. Cellular senescence increases expression of bacterial ligands in the lungs and is positively correlated with increased susceptibility to pneumococcal pneumonia. Aging Cell 10, 798-806.
28. Reilly, S. M., et al. Nuclear receptor corepressor SMRT regulates mitochondrial oxidative metabolism and mediates aging-related metabolic deterioration. Cell metabolism 12, 643-653 (2010).
29. Sung, B., Park, S., Yu, B. P. & Chung, H. Y. Modulation of PPAR in aging, inflammation, and calorie restriction. J Gerontol A Biol Sci Med Sci 59, 997-1006 (2004).
30. Arredouani, M., et al. The scavenger receptor MARCO is required for lung defense against pneumococcal pneumonia and inhaled particles. J Exp Med 200, 267-272 (2004).
31. Lonnbro, P., Nordenfelt, P. & Tapper, H. Isolation of bacteria-containing phagosomes by magnetic selection. BMC Cell Biol 9, 35 (2008).
32. Sokolovska, A., Becker, C. E. & Stuart, L. M. Measurement of phagocytosis, phagosome acidification, and intracellular killing of Staphylococcus aureus. Curr Protoc Immunol Chapter 14, Unit14 30 (2012).
33. Barak, Y., et al. Effects of peroxisome proliferator-activated receptor delta on placentation, adiposity, and colorectal cancer. Proc Natl Acad Sci USA 99, 303-308 (2002).
34. Barak, Y., et al. PPAR gamma is required for placental, cardiac, and adipose tissue development. Mol Cell 4, 585-595 (1999).
35. Blasi, E., et al. Selective immortalization of murine macrophages from fresh bone marrow by a raf/myc recombinant murine retrovirus. Nature 318, 667-670 (1985).
36. Gandino, L. & Varesio, L. Immortalization of macrophages from mouse bone marrow and fetal liver. Exp Cell Res 188, 192-198 (1990).
37. Brodsky, I. E., Ghori, N., Falkow, S. & Monack, D. Mig-14 is an inner membrane-associated protein that promotes Salmonella typhimurium resistance to CRAMP, survival within activated macrophages and persistent infection. Mol Microbiol 55, 954-972 (2005).
38. Kohler, J., et al. NADPH-oxidase but not inducible nitric oxide synthase contributes to resistance in a murine Staphylococcus aureus Newman pneumonia model. Microbes Infect 13, 914-922.
39. Cooney, G. J., et al. Improved glucose homeostasis and enhanced insulin signalling in Grb14-deficient mice. EMBO J. 23, 582-593 (2004).
40. Molero, J. C., Whitehead, J. P., Meerloo, T. & James, D. E. Nocodazole inhibits insulin-stimulated glucose transport in 3T3-L1 adipocytes via a microtubule-independent mechanism. The Journal of biological chemistry 276, 43829-43835 (2001).
   All patents and other publications identified in the specification and examples are expressly incorporated herein by reference for all purposes. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

1. A method of treating, ameliorating, or preventing an infection, a respiratory condition or one or more symptoms thereof in a subject, said method comprising administering to said subject a therapeutically effective amount of a peroxisome proliferator activated receptor PPAR agonist to a subject in need thereof.

2.-34. (canceled)

35. The method of claim 1, wherein the PPAR agonist is a PPARδ or a PPARγ agonist.

36. The method of claim 1, further comprising co-administering a prophylactic or therapeutic agent to the subject.

37. The method of claim 36, wherein the prophylactic or therapeutic agent is not a PPAR agonist.

38. The method of claim 36, wherein the prophylactic or therapeutic agent is an immunomodulatory agent, an anti-inflammatory agent, an anti-viral agent, an antibiotic, an antifungal agent or a mast cell modulator.

39. The method of claim 1, wherein the subject is an immunocompromised or immunosuppressed subject.

40. The method of claim 1, wherein the subject has bronchopulmonary dysplasia, congenital heart disease, cystic fibrosis or acquired or congenital immunodeficiency.

41. The method of claim 1, wherein the infection is a viral infection, a bacterial infection, a fungal infection, or a parasite infection.

42. The method of claim 1, wherein the infection is a respiratory, peritoneum, urinary tract, or gut infection.

43. The method of claim 42, wherein the respiratory infection is selected from the group consisting of upper respiratory infection, influenza, croup, respiratory syncytial virus, bronchitis, bronchiolitis and pneumonia.

44. The method of claim 1 wherein the PPAR agonist is a compound of formula (I): 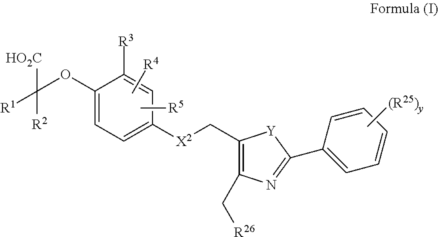
wherein:
R¹ and R² are independently hydrogen or C₁₋₃alkyl;
X² is O, S, or CH₂;
R³, R⁴, and R⁵ are independently H, C₁₋₃alkyl, OCH₃, CF₃, OCF₃, CN, allyl, or halogen;
Y is S or O;
each R²⁵ is independently CH₃, OCH₃, CF₃, or halogen;
each R²⁶ is independently for each occurrence 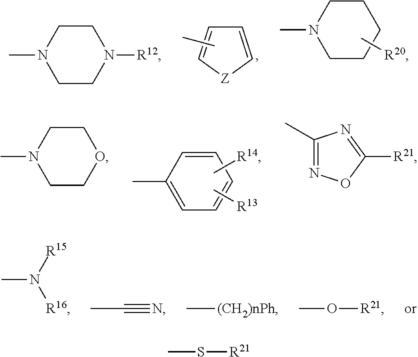
R¹² is independently for each occurrence C₁₋₆alkyl, C₁₋₆alkylenearyl, 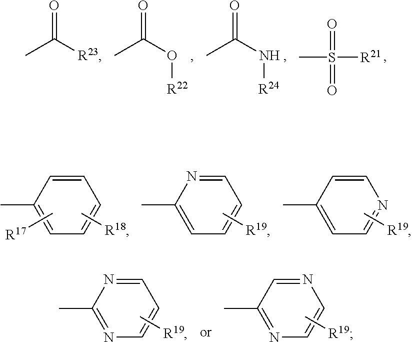
R¹³ and R¹⁴ are independently hydrogen, halogen, CN, perfluoroC₁₋₆alkyl, perfluoro-O—C₁₋₆alkyl, C₁₋₆alkyl, —OC₁₋₆alkyl, —C₁₋₆alkyleneOC₁₋₆alkyl, —SC₁₋₆alkyl, or aryl;
R¹⁵ and R¹⁶ are independently hydrogen, C₁₋₆alkyl, C₃₋₆cycloalkyl optionally substituted with 1 or 2 C₁₋₃alkyl groups, or R¹² as defined above;
R¹⁷ and R¹⁸ are independently hydrogen, halogen, hydroxy, —CN, C₁₋₆alkyl, C₁₋₆ perfluoroalkyl, C₁₋₆acyl, —OC₁₋₆alkyl, perfluoroOC₁₋₆alkyl, or C₁₋₆hydroxyalkyl;
R¹⁹ is independently for each occurrence hydrogen or C₁₋₆alkyl;
R²⁰ is independently for each occurrence C₁₋₆alkyl, aryl, —OC₁₋₆alkyl, hydroxy, C₁₋₆ hydroxyalkyl, or 1-alkoxyC₁₋₆alkyl;
R²¹ is independently for each occurrence C₁₋₆alkyl, —C₁₋₆alkylenearyl, aryl, or aryl-heteroaryl;
R²² is independently for each occurrence independently for each occurrence C₁₋₆alkyl, aryl, or —C₁₋₆alkylenearyl;
R²³ is C₁₋₆alkyl, C₃₋₆cycloalkyl, or aryl;
R²⁴ is independently for each occurrence C₁₋₆alkyl, C₁₋₆alkylenearyl, C₃₋₆cycloalkyl, or aryl;
Z is independently for each occurrence O, N, or S (note that when Z is N, the depicted bond can be attached to the nitrogen in the ring as well as any of the carbons in the ring);
y is 0, 1, 2, 3, 4, or 5; and
n is 1, 2, or 3; and
pharmaceutically acceptable salts, solvates, or hydrolyzable esters thereof.

45. The method of claim 44, wherein the compound of formula (I) is: 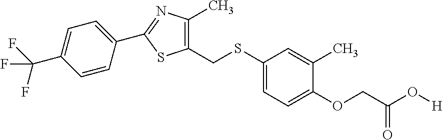 [SMILES: Cc1cc(SCc2sc(-c3ccc(C(F)(F)F)cc3)nc2C)ccc1OCC(=O)O]
(GW501516).

46. The method of claim 1, wherein the PPAR agonist is selected from the group consisting of 1-methylethyl 2-(4-(4-chlorobenzoyl)phenoxy)-2-methylpropionate (fenofibrate), ((4-chloro-6-((2,3-dimethylphenyl)amino)-2-pyrimidinyl)thio)acetic acid (WY-14643), (4-(3-(4-acetyl-3-hydroxy-2-propyl)phenoxy)propoxyphenoxy)acetic acid (L-165041), (4-(((2-(3-fluoro-4-(trifluoromethyl)phenyl)-4-methyl-5-thiazolyl)methyl)thio)-2-methylphenoxy)acetic acid (GW-0742), 2-methyl-4-((2R)-2-(3-methyl-5-(4-(trifluoromethyl)phenyl)-2-thienyl)propoxy)-benzenepropionic acid, 2-ethyl-2-(4-(4-(4-(4-methoxyphenyl)-piperazin-1-yl)-2-(4-trifluoromethyl-phenyl)-thiazol-5-ylmethylsulfanyl)-phenoxy)butyric acid (GSK-677954), (4-(3-(3-phenyl-7-propyl-benzofuran-6-yloxy)-propylsulfanyl)-phenyl)acetic acid (L-796449), 2-(4-(3-(1-(2-(2-chloro-6-fluoro-phenyl)-ethyl)-3-(2,3-dichloro-phenyl)-ureido)-propyl)-phenoxy)-2-methylpropionic acid (GW-2433), 2-{2-methyl-4-[({4-methyl-2-[4-(trifluoromethyl)phenyl]-1,3-thiazol-5-yl}methyl)sulfanyl]phenoxy}acetic acid, 2-{2-methyl-4-[({4-methyl-2-[4-(trifluoromethyl)phenyl]-1,3-oxazol-5-yl}methyl)sulfanyl]phenoxy}acetic acid, methyl 2-{4-[({4-methyl-2-[4-(trifluoromethyl)phenyl]-1,3-thiazol-5-yl}methyl)sulfanyl]phenoxy}acetate, 2-{4-[({4-methyl-2-[4-(trifluoromethyl)phenyl]-1,3-thiazol-5-yl}methyl)sulfanyl]phenoxy}acetic acid, (E)-3-[2-methyl-4-({4-methyl-2-[4-(trifluoromethyl)phenyl]-1,3-thiazol-5-yl}methoxy)phenyl]-2-propenoic acid, 2-{3-chloro-4-[({4-methyl-2-[4-(trifluoromethyl)phenyl]-1,3-thiazol-5-yl}methyl)sulfanyl]phenyl}acetic acid, and metabolites of arachidonic acid, e.g., 4-hydroxy-2-nonenal (4-HNE), 4-hydroxydodeca-(2E,6Z)-dienal (4-HDDE), rosiglitazone, pioglitazone, troglitazone, netoglitazone (also known as MCC-555 or isaglitazone or neoglitazone), 5-BTZD, farglitazar, 677954 (GlaxoSmithKline), PLX204 (Plexxikon), LY 519818, L-783483, L-165461, L-16504, and the like.

47. The method of claim 1, wherein the infection is Streptococcus pneumonia, Staphylococcus aureus, Haemophilus influenzae, Chlcanda pneumoniae, C. psittaci, C. trachomatis, Moraxella (Branhamella) catarrhalis, Legionella pneumophila, or Klebsiella penumoniae infection.

48. A method of treating or ameliorating pneumonia in a human subject suffering therefrom, said method comprising administering to said human subject an effective amount of a PPAR agonist.

49. The method of claim 48, wherein the PPAR agonist is a PPARδ or a PPARγ agonist.

50. A composition for treating, ameliorating and/or preventing an infection in a subject comprising an effective amount of at least one effective PPAR agonist.

51. The composition of claim 50, further comprising at least one of an immunomodulatory agent, an anti-inflammatory agent, an anti-viral agent, an antibiotic, an antifungal agent or a mast cell modulator.

52. The composition of claim 50, wherein the PPAR agonist is a PPARδ or a PPARγ agonist.

53. The composition of claim 50, further comprising a pharmaceutically acceptable carrier.
